(19) United States
(12) Patent Application Publication
(10) Pub. No.: US 2023/0257820 A1
(43) Pub. Date: Aug. 17, 2023

(54) METHODS OF DETERMINING COVID-19 MORTALITY RISK

(71) Applicants: Ashley Steed, St. Louis, MO (US); Junedh Amrute, St. Louis, MO (US)

(72) Inventors: Ashley Steed, St. Louis, MO (US); Junedh Amrute, St. Louis, MO (US)

(73) Assignee: Washington University, St. Louis, MO (US)

(21) Appl. No.: 18/168,955

(22) Filed: Feb. 14, 2023

Related U.S. Application Data
(60) Provisional application No. 63/309,782, filed on Feb. 14, 2022.

(51) Int. Cl. C12Q 1/6883

(52) U.S. Cl. CPC C12Q 1/6883; C12Q 2600/118; C12Q 2600/158

(57) ABSTRACT

Among the various aspects of the present disclosure is the provision of detecting COVID-19 severity. One aspect provides a method of predicting survival in subjects having COVID-19 (e.g., critical COVID-19) comprising single-cell RNA sequencing and Cellular Indexing of Transcriptomes and Epitomes by sequence mapping to elucidate cell type specific transcriptional signatures. Another aspect provides for a method of predicting COVID-19 infection survival comprising detecting activation of antibody processing, early activation response, and/or cell cycle regulation pathways most prominent within B-, T-, and/or NK-cell subsets. Yet another aspect provides for a method of predicting mortality in a subject having COVID-19, comprising detecting interferon signaling and antigen presentation pathways within cDC2 cells, CD14 monocytes, and/or CD16 monocytes. In some embodiments, the method comprises detecting cell specific differential gene expression and machine learning to predict mortality using single cell transcriptomes. In some embodiments, the subject is prioritized for treatment of COVID-19.

### CROSS-REFERENCE TO RELATED APPLICATIONS

Not applicable.

### STATEMENT REGARDING FEDERALLY SPONSORED RESEARCH OR DEVELOPMENT

Not applicable.

### MATERIAL INCORPORATED-BY-REFERENCE

This application claims priority from U.S. Provisional Application Ser. No. 63/309,782 filed on 14 Feb. 2022, which is incorporated herein by reference in its entirety.

### FIELD

The present disclosure generally relates to determining risk of mortality from critical COVID-19.

### SUMMARY

In an aspect of the present disclosure, a method of determining a risk of mortality from critical COVID-19 for a subject in need thereof is provided. The method comprises: obtaining a biological sample from the subject; measuring an expression level of at least two genes selected from: APOBEC3A, AREG, B2M, BST2, CCL4, CD52, CD74, CDKN1A, CEBPB, CEBPD, CFD, CRIP1, CTSW, CYBA, DDIT4, DDIT4, EEF1A1, EEF1B2, EEF1G, EGR1, EIF3L, EMP3, EPSTI1, GNLY, GZMB, GZMH, GZMI, H1FX, H3F3B, HIST1H1E, HLA-A, HLA-C, HLA-DPB1, HLA-DQA1, HLA-DQB1, HLA-DRB5, HSPB1, IER2, IFI27, IFI44L, IFI6, IFIT3, IFITM1, IFITM2, IFITM3, IGHG1, IL7R, IRF1, IRF7, ISG1, ISG15, ISG20, JUN, JUNB, KLF2, KLF6, LGALS1, LGALS9, LY6E, MAFB, MALAT1, MT2A, MT-CO1, MX1, NFKBIA, OAS1, PLAC3, PNRC1, PPP1R15A, PRF1, PTGDS, RHOB, RPS2, RPS4Y1, SAT1, SESN1, TMSB4X, TPT1, TRBV4-1, TRGV9, TSC22D3, TXNDC5, TXNIP, UBE2L6, XAF1, XCL2, YPEL3, or ZFP36; computing a gene expression score from the expression level of the at least two genes; comparing the gene expression score to a reference score computed from a reference sample; and determining the subject has a high risk of mortality from critical COVID-19 when the gene expression score is significantly different from the reference score; or determining the subject has a low risk of mortality from critical COVID-19 when the gene expression score is not significantly different from the reference score.

In some embodiments, the biological sample is a blood sample, and/or the expression level is measured from an immune cell in the biological sample. In some embodiments, the immune cell is a CD14 monocyte, CD16 monocyte, a type II conventional dendritic cell (cDC2), a B cell, a plasmablast, a CD4 T-cell with cytotoxic activity (CD4 CTL), a CD8 T-cell, a natural killer (NK) cell, a NK proliferating cell, or a mucosal-associated invariant T (MAIT) cell.

In some embodiments, (i) the immune cell is a CD14 monocyte and the at least two genes are selected from IFITM1, IFITM3, JUNB, IFI27, LGALS1, MAFB, NFKBIA, and CEBPD; (ii) the immune cell is a CD16 monocyte and the at least two genes are selected from IFITM2, IFI30, HLA-DPB1, IFI27, HLA-DRB5, CD52, KLF2, and HLA-DQA1; or (iii) the immune cell is a cDC2 cell and the at least two genes are selected from OAS1, IFITM3, EGR1, HLA-DRB5, EEF1A1, and RPS4Y1. In some embodiments, (i) the immune cell is a CD8 T-cell and the at least two genes are selected from CTSW, GZMH, MT-CO11, GZMB, GNLY, TMSB4X, TRBV4-1, TRGV9, TPT1, TMSB4X, KLF2, and RPS4Y1; (ii) the immune cell is a CD4 CTL and the at least two genes are selected from TMSB4X, YPEL3, H1FX, KLF2, RPS4Y1, CDKN1A, CTSW, RPS2, ISG20, IFITM1, IFIT3, LY6E, XAF1, IFI6, and ISG15, (iii) the immune cell is an NK cell and the at least two genes are selected from CCL4, PTGDS, ISG15, GZMB, IFI6, and GNLY; or (iv) the immune cell is a B cell and the at least two genes are selected from IFIT3, IFITM1, TSC22D3, SESN1, KLF2, HLA-DRB5, MALAT1, H1FX, IGHG1, HLA-DQB1, HLA-DRB5, ISG15, IFI6, and TXNDC5.

In some embodiments, the B cell is an B intermediate cell, a B memory cell, or a B naïve cell. In some embodiments, the subject is hospitalized. In some embodiments, the reference sample is derived from a healthy control subject or a subject having survived critical COVID. In some embodiments, the method further comprises administering a treatment based at least in part on the gene expression score.

In another aspect of the present disclosure, a method for monitoring critical COVID-19 in a subject in need thereof. The method comprises: obtaining a biological sample from the subject; measuring a first expression level of at least two genes selected from: APOBEC3A, AREG, B2M, BST2, CCL4, CD52, CD74, CDKN1A, CEBPB, CEBPD, CFD, CRIP1, CTSW, CYBA, DDIT4, DDIT4, EEF1A1, EEF1B2, EEF1G, EGR1, EIF3L, EMP3, EPSTI1, GNLY, GZMB, GZMH, GZMI, H1FX, H3F3B, HIST1H1E, HLA-A, HLA-C, HLA-DPB1, HLA-DQA1, HLA-DQB1, HLA-DRB5, HSPB1, IER2, IFI27, IFI44L, IFI6, IFIT3, IFITM1, IFITM2, IFITM3, IGHG1, IL7R, IRF1, IRF7, ISG1, ISG15, ISG20, JUN, JUNB, KLF2, KLF6, LGALS1, LGALS9, LY6E, MAFB, MALAT1, MT2A, MT-CO1, MX1, NFKBIA, OAS1, PLAC3, PNRC1, PPP1R15A, PRF1, PTGDS, RHOB, RPS2, RPS4Y1, SAT1, SESN1, TMSB4X, TPT1, TRBV4-1, TRGV9, TSC22D3, TXNDC5, TXNIP, UBE2L6, XAF1, XCL2, YPEL3, or ZFP36; computing a first gene expression score from the first expression level of the at least two genes; and subsequently measuring a second expression level of the at least two genes and computing a second gene expression score from the second expression level of the at least two genes; wherein a change in gene expression score is computed as a difference between the first expression score and the second expression score, and indicates a change in the subject's risk of mortality from critical COVID-19.

In some embodiments, the biological sample is a blood sample, and/or the expression level is measured from an immune cell in the biological sample. In some embodiments, the immune cell is a CD14 monocyte, CD16 monocyte, a type II conventional dendritic cell (cDC2), a B cell, a plasmablast, a CD4 T-cell with cytotoxic activity (CD4 CTL), a CD8 T-cell, a natural killer (NK) cell, a NK proliferating cell, or a mucosal-associated invariant T (MAIT) cell.

In some embodiments, (i) the immune cell is a CD14 monocyte and the at least two genes are selected from IFITM1, IFITM3, JUNB, IFI27, LGALS1, MAFB, NFKBIA, and CEBPD; (ii) the immune cell is a CD16 monocyte and the at least two genes are selected from IFITM2, IFI30, HLA-DPB1, IFI27, HLA-DRB5, CD52, KLF2, and HLA-DQA1; or (iii) the immune cell is a cDC2 cell and the at least two genes are selected from OAS1, IFITM3, EGR1, HLA-DRB5, EEF1A1, and RPS4Y1. In some embodiments, (i) the immune cell is a CD8 T-cell and the at least two genes are selected from CTSW, GZMH, MT-CO1, GZMB, GNLY, TMSB4X, TRBV4-1, TRGV9, TPT1, TMSB4X, KLF2, and RPS4Y1; (ii) the immune cell is a CD4 CTL and the at least two genes are selected from TMSB4X, YPEL3, H1FX, KLF2, RPS4Y1, CDKN1A, CTSW, RPS2, ISG20, IFITM1, IFIT3, LY6E, XAF1, IFI6, and ISG15, (iii) the immune cell is an NK cell and the at least two genes are selected from CCL4, PTGDS, ISG15, GZMB, IFI6, and GNLY; or (iv) the immune cell is a B cell and the at least two genes are selected from IFIT3, IFITM1, TSC22D3, SESN1, KLF2, HLA-DRB5, MALAT1, H1FX, IGHG1, HLA-DQB1, HLA-DRB5, ISG15, IFI6, and TXNDC5.

In some embodiments, the B cell is an B intermediate cell, a B memory cell, or a B naïve cell. In some embodiments, the subject is hospitalized. In some embodiments, the method further comprises administering a treatment to the subject based at least in part on the change in the subject's risk of mortality from critical COVID-19. In some embodiments, the method further comprises administering a treatment to the subject based at least in part on the change in gene expression score.

Other objects and features will be in part apparent and in part pointed out hereinafter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a table showing demographics and clinical characteristics of patient samples selected for sequencing in accordance with the present disclosure.

FIG. 2A-FIG. 2B is an exemplary embodiment showing common laboratory evaluation in critical COVID-19 patients by survival outcome in accordance with the present disclosure. Mann-Whitney statistical tests were performed. ** denotes p<0.01 and ns denotes not significant.

FIG. 3 is an exemplary embodiment showing single-cell transcriptomic mapping of PBMCs during critical COVID-19 in accordance with the present disclosure. FIG. 3 is a schematic showing study design.

FIG. 4A-FIG. 4D is an exemplary embodiment showing scRNA-sequencing quality control metrics post-filtering by number of genes >200 and <5000 and percentage mitochondrial sequencing reads <10 in accordance with the present disclosure. FIG. 4A is a graph showing the number of UMI counts by cell type annotation. FIG. 4B is a kernel density plot using Nebulosa for FIG. 4A. FIG. 4C is a graph showing the number of genes by cell type annotation. FIG. 4D is a graph showing mitochondrial sequencing read percentage by cell type annotation.

FIG. 5A-FIG. 5D is an exemplary embodiment showing Azimuth mapping to PBMC CITE-seq reference in accordance with the present disclosure. FIG. 5A-FIG. 5D include UMAP embedding plots of PBMC scRNA sequencing profiles mapped onto a PBMC CITE-seq reference derived UMAP space via Azimuth with (FIG. 5A) imputed cell annotations, (FIG. 5B) disease status, (FIG. 5C) time and survival outcome, and (FIG. 5D) sample ID.

FIG. 6A-FIG. 6B is an exemplary embodiment showing single-cell transcriptomic mapping of PBMCs during critical COVID-19 in accordance with the present disclosure. FIG. 6A includes UMAP embedding plots of scRNA sequencing profiles of 199,097 cells with cluster annotations derived from Azimuth mapping, a CITE-sequencing reference dataset. FIG. 6B includes UMAP embedding plots for each of the following conditions: Control, Alive Day 0, Alive Day 7, Deceased Day 0, and Deceased Day 7 (n=6 each).

FIG. 7A-FIG. 7H include UMAP embedding plots of Azimuth mapping prediction scores for all cell types (FIG. 7A, FIG. 7B, FIG. 7C, FIG. 7D, FIG. 7E, FIG. 7F, FIG. 7G, and FIG. 7H) annotated on the reference derived UMAP space in accordance with the present disclosure.

FIG. 8A-FIG. 8B is an exemplary embodiment showing single-cell transcriptomic mapping of PBMCs during critical COVID-19 in accordance with the present disclosure. FIG. 8A is a heatmap of the top marker gene for each cell type annotated. FIG. 8B shows Azimuth mapping cell type prediction scores.

FIG. 9A-FIG. 9D is an exemplary embodiment showing recomputed UMAP with merged query and reference after Seurat multi-modal reference mapping in accordance with the present disclosure. FIG. 9A-FIG. 9D include de novo UMAP embedding plots of PBMC scRNA sequencing profiles mapped via Azimuth with (FIG. 9A) reference embedding, (FIG. 9B) disease status, (FIG. 9C) time and survival outcome, and (FIG. 9D) sample ID.

FIG. 10A-FIG. 10C include box plots showing percentage of cell types (FIG. 10A, FIG. 10B, and FIG. 10C) annotated in accordance with the present disclosure. Mann-Whitney statistical tests were performed. * denotes p<0.05 and ** denotes p<0.01.

FIG. 11A-FIG. 11D is an exemplary embodiment showing single-cell transcriptomics reveal number and magnitude of differential gene expression in specific cell populations during the evolution of critical COVID-19 in accordance with the present disclosure. FIG. 11A-FIG. 11D include graphs showing number of differentially expressed genes (adjusted p-value<0.05 and log₂ FC>0.58) in annotated populations between (FIG. 11A) Control versus Day 0, (FIG. 11B) Control versus Day 7, (FIG. 11C) Alive vs Deceased Day 0, and (FIG. 11D) Alive vs. Deceased Day 7. Dot plots within each subfigure show magnitude of fold-change for each cell type in the same order as the corresponding bar plot. Red dots in dot plots denote differentially expressed genes that reached statistical significance. Differential expression analysis was performed using the default Seurat non-parametric Wilcoxon rank-sum test.

FIG. 12A-FIG. 12I is an exemplary embodiment showing B-cell subsets display the strongest transcriptional differences between alive and deceased patients on day 7 in accordance with the present disclosure. FIG. 12A includes UMAP embedding plots of B-naive, B-intermediate, B-memory cells, and plasmablasts for the following sample conditions: control, Alive day 7, and Deceased day 7. FIG. 12B includes UMAP embedding plots of B-cell subsets with imputed CITEseq surface-protein expression for canonical markers. FIG. 12C is a hierarchical clustering heatmap of average normalized gene expression for statistically significant differentially expressed genes (adjusted p-value<0.05 and log₂ FC>0.50) between Alive and Deceased day 7 B-naive, B-intermediate, and B-memory cells. FIG. 12D is a Venn diagram of activation genes from FIG. 12C denoting overlapping signatures across B-cell subsets. FIG. 12E shows B-cell activation signature z-score for all genes in FIG. 12D overlaid on UMAP embedding plots of B-cells (left) and quantified (right). FIG. 12F is a Venn diagram of cell-cycle regulation genes from FIG. 12C denoting overlapping signatures across B-cell subsets. FIG. 12G shows B-cell activation signature z-score for all genes in FIG. 12F overlaid on UMAP embedding plots of B-cells (left) and quantified (right). FIG. 12H is a hierarchical clustering heatmap of average normalized gene expression for statistically significant differentially expressed genes (adjusted p-value<0.05 and log₂ FC>0.50) between Alive and Deceased day 7 plasmablasts (left) and antibody processing gene z-scores overlaid on UMAP embedding plots of plasmablasts (middle) and quantified (right). FIG. 12I shows interferon signaling gene z-scores overlaid on UMAP embedding of B-cell subsets with quantification in B-naïve cells (left) and plasmablasts (right). On all heatmaps blue (low) to red (high) expression. 3261 Control, 5270 Alive day 7, and 2397 Deceased day 7 B-cells were examined across 18 patients. 135 Control, 804 Alive Day 7, and 287 Deceased Day 7 Plasmablasts were examined across 18 patients. Ordinary one-way ANOVA statistical tests were used for each comparison. ** denotes p<0.01, **** denotes p<0.0001, and ns denotes not significant.

FIG. 13A-FIG. 13E is an exemplary embodiment showing day 7 functional gene set scores calculated for control and COVID-19 cohorts in CD8 Naïve, NK/NK Proliferating, MAIT, and cDC2 cells in accordance with the present disclosure. FIG. 13A is a hierarchical clustering heatmap (left) of average normalized gene expression for differentially expressed genes (adjusted p-value<0.05 and log₂ FC>0.50) between Control, Alive day 7, and Deceased day 7 CD8 naïve cells and their ISG z-scores, cell cycle regulation gene z-scores, and activation gene z-scores overlaid on UMAP embedding plots (right, top) with quantification (right, bottom). FIG. 13B is a hierarchical clustering heatmap (left) of average normalized gene expression for differentially expressed genes (adjusted p-value<0.05 and log2 FC>0.50) between Control, Alive day 7, and Deceased day 7 MAIT cells and their ISG z-scores and cell cycle regulation gene z-scores overlaid on UMAP embedding plots (right, top) with quantification (right, bottom). FIG. 13C includes UMAP embedding plots of NK and NK proliferating cells of Control, Alive day 7, and Deceased day 7. FIG. 13D is a hierarchical clustering heatmap (left) of average normalized gene expression for differentially expressed genes (adjusted p-value<0.05 and log₂ FC>0.50) between Control, Alive day 7, and Deceased day 7 NK and NK proliferating cells and their ISG z-scores, cell cycle regulation gene z-scores, and activation gene z-scores overlaid on UMAP embedding plots (right, top) with quantification (right, bottom). FIG. 13E is a hierarchical clustering heatmap (left) of average normalized gene expression for differentially expressed genes (adjusted p-value<0.05 and log₂ FC>0.50) between Control, Alive day 7, and Deceased day 7 cDC2 cells and their ISG z-scores and cell cycle regulation gene z-scores overlaid on UMAP embedding plots (right, top) with quantification (right, bottom). On all heatmaps blue (low) to red (high) expression. 982 Control, 787 Alive day 7, and 1,647 Deceased day 7 CD8 Naïve cells were examined across 18 patients. 235 Control, 122 Alive day 7, and 397 Deceased day 7 MAIT cells were examined across 18 patients. 4,273 Control, 6,459 Alive day 7, and 2,338 Deceased day 7 NK/NK-Proliferating cells were examined across 18 patients. 553 Control, 130 Alive Day 7, and 361 Deceased day 7 cDC2 cells were examined across 18 patients. Ordinary one-way ANOVA statistical tests were used for each comparison. ** denotes p<0.01, *** denotes p<0.001, **** denotes p<0.0001, and ns denotes not significant.

FIG. 14A-FIG. 14F is an exemplary embodiment showing innate immune cells dominate early peripheral immune responses and predict survival in critical COVID-19 in accordance with the present disclosure. FIG. 14A is a UMAP embedding plot of CD14 monocytes, CD16 monocytes, and cDC2 cells for the following sample conditions: Control, Alive day 0, and Deceased day 0. FIG. 14B is a hierarchical clustering heatmap of average normalized gene expression for statistically significant differentially expressed genes (adjusted p-value<0.05 and log₂ FC>0.50) between Control, Alive day 0 and Deceased day 0 CD14 monocytes, CD16 monocytes, and cDC2 cells. FIG. 14C-FIG. 14F include UMAP embedding plots and quantification for Control, Alive day 0, and Deceased day 0 samples for (FIG. 14C) inflammatory activation gene set z-scores, (FIG. 14D) antigen-presentation gene set z-scores, (FIG. 14E) ISG set z-scores, and (FIG. 14F) protein synthesis gene set z-scores from genes in FIG. 14B. All comparisons were statistically significant (p<0.0001) except the ones marked n.s.

FIG. 15A-FIG. 15E is an exemplary embodiment showing day 0 functional gene set scores calculated for control and COVID-19 cohorts in B cell subsets, CD4 CTL, and NK/NK proliferating cells in accordance with the present disclosure. FIG. 15A is a UMAP embedding plot of B-cell subsets in Control, Alive day 0 and Deceased day 0 samples. FIG. 15B is a hierarchical clustering heatmap (left) of average normalized gene expression for differentially expressed genes (adjusted p-value<0.05 and log₂ FC>0.50) between Control, Alive day 0, and Deceased day 0 B-cell subsets and their cell cycle regulation gene z-scores and activation gene z-scores overlaid on UMAP embedding plots (right, top) with quantification (right, bottom). FIG. 15C is a plot of SARS-CoV-2 IgG II serology in critical COVID-19 patients at day 0 split by outcome (left) and fold change (day 7/day 0) split by outcome (right). FIG. 15D is a UMAP embedding plot of CD4 CTL, NK, and NK proliferating cells in Control, Alive day 0 and Deceased day 0 samples. FIG. 15E is a hierarchical clustering heatmap (left) of average normalized gene expression for differentially expressed genes (adjusted p-value<0.05 and log₂ FC>0.50) between Control, Alive day 0, and Deceased day 0 CD4 CTL, NK, and NK proliferating cells and their ISG z-scores and activation gene z-scores overlaid on UMAP embedding plots (right, top) with quantification (right, bottom). On all heatmaps blue (low) to red (high) expression. 3,261 Control, 4,249 Alive day 0, and 2,163 Deceased day 0 B naïve/intermediate/memory cells were examined across 18 patients. 5,511 Control, 4,082 Alive day 0, and 3,150 Deceased day 0 CD4 CTL/NK/NK Proliferating cells were examined across 18 patients. Ordinary one-way ANOVA statistical tests were used for each comparison. * denotes p<0.05, **** denotes p<0.0001, and ns denotes not significant.

FIG. 16A-FIG. 16E is an exemplary embodiment showing random forest classification hyperparameter tuning grid search with number of estimators and max feature (sqrt or log₂) assessment using 10-fold cross validation with 5 trials (50 repeats) in accordance with the present disclosure. Mean accuracy and standard deviation plotted for each cell type (FIG. 16A, FIG. 16B, FIG. 16C, FIG. 16D, and FIG. 16E) annotated.

FIG. 17A-FIG. 17B is an exemplary embodiment showing innate immune cells dominate early peripheral immune responses and predict survival in critical COVID-19 in accordance with the present disclosure. FIG. 17A is a graph showing random forest classifier model survival prediction accuracy using 3000 highly variable gene normalized counts in all cell types with at least 100 cells. Red boxed cell types are those with a prediction accuracy of >80%. FIG. 17B includes graphs showing ranked feature importance score from random forest classifier model with key genes annotated in CD14 monocytes (top), CD16 monocytes (middle), and cDC2 cells (bottom).

FIG. 18A-FIG. 18D is an exemplary embodiment showing random forest classifier predicted feature importance scores calculated for key cell types in accordance with the present disclosure. FIG. 18A-FIG. 18D include graphs showing random forest classifier ranked feature importance score for key genes annotated in (FIG. 18A) CD8 T-cell subsets, (FIG. 18B) CD4 T-cell subsets, (FIG. 18C) NK cells, and (FIG. 18D) B-cell subsets and plasmablasts.

FIG. 19A-FIG. 19C is an exemplary embodiment showing innate immune cells dominate early peripheral immune responses and predict survival in critical COVID-19 in accordance with the present disclosure. FIG. 19A is a global UMAP embedding plot of the top 100 predictive features in CD14 monocytes, CD16 monocytes, and cDC2 cells for Control (top), Alive day 0 (middle), and Deceased day 0 (bottom). FIG. 19B is a Venn diagram of overlapping genes from the top predictive features for the CD14 monocytes random forest classifier, statistically significant differentially expressed genes between Alive day 0 and Deceased day 0 CD14 monocytes, and statistically significant differentially expressed genes between Control and day 0 CD14 monocytes. FIG. 19C includes a UMAP embedding plot from FIG. 14A of Control (left, top), Alive day 0 (left, middle), and Deceased day 0 (left, bottom) for four overlapping genes (CEBPD, MAFB, IFITM3, and LGALS1) identified from FIG. 19B with z-score quantification (right). On all heatmaps blue (low) to red (high) expression. 12,044 Control, 8530 Alive day 0, and 5385 Deceased day 0 CD14 Monocytes were examined across 18 patients. 1694 Control, 1559 Alive day 0, and 1216 Deceased day 0 CD16 Monocytes were examined across 18 patients. 553 Control, 108 Alive day 0, and 104 Deceased day 0 cDC2 cells were examined across 18 patients. Ordinary one-way ANOVA statistical tests were used for each comparison. **** denotes p<0.0001.

FIG. 20A-FIG. 20E is an exemplary embodiment showing IL-6 pathway enrichment in monocytes and dendritic cells in accordance with the present disclosure. FIG. 20A-FIG. 20B include UMAP embedding plots of (FIG. 20A) CEBPB and (FIG. 20B) LGALS3 expression in CD14 monocytes, CD16 monocytes, and cDC2 cells by time and outcome. FIG. 20C-FIG. 20E include hierarchical clustering heatmaps (left) of average normalized gene expression for IL-6 signaling pathway genes and quantification (right) of IL-6 signaling pathway scores in (FIG. 20C) CD14 monocytes, (FIG. 20D) CD16 monocytes, and (FIG. 20E) cDC2 cells. On all heatmaps blue (low) to red (high) expression. 12,044 Control, 8,530 Alive day 0, and 5,385 Deceased day 0 CD14 Monocytes were examined across 18 patients. 1,694 Control, 1,559 Alive Day 0, and 1,216 Deceased day 0 CD16 Monocytes were examined across 18 patients. 553 Control, 108 Alive day 0, and 104 Deceased day 0 cDC2 cells were examined across 18 patients. Ordinary one-way ANOVA statistical tests were used for each comparison. **** denotes p<0.0001, and ns denotes not significant.

FIG. 21A-FIG. 21D shows an exemplary embodiment of cross-validation of random forest predicted gene signature in an independent cohort of critical COVID in accordance with the present disclosure. FIG. 21A is a table showing number of CD14 monocytes, CD16 monocytes, and cDC2 cells sequenced by outcome in this study and that by Liu et al. FIG. 21B is a graph showing ranked feature importance score from random forest classifier model with key genes annotated in CD14 monocytes in the critically ill cohort in Liu et al. FIG. 21C-FIG. 21D include graphs showing z-score for CEBPD, MAFB, IFITM3, and LGALS1 in CD14 monocytes from Liu et al. (FIG. 21C) by disease severity pooled at day 0 and (FIG. 21D) by survival outcome at day 0 (healthy controls, n=14, and critically ill patients, n=25; 21 alive and 4 deceased). 13,464 Control, 1297 Moderate day 0, 2798 Severe day 0, and 20,775 Critical day 0 CD14 Monocytes were examined. Ordinary one-way ANOVA statistical tests were used for each comparison. ns denotes not significant and **** denotes p<0.0001.

### DETAILED DESCRIPTION

The present disclosure is based, at least in part, on the discovery of cell type specific transcriptional signatures (e.g., gene expression scores calculated from measured expression levels of at least two genes) that associate with and predict survival in critical COVID-19. As shown herein, single-cell RNA sequencing (scRNA-seq) of peripheral blood mononuclear cells (PBMCs) from patients with critical COVID-19 who survived or died was mapped onto a Cellular Indexing of Transcriptomes and Epitomes by sequencing (CITE-seq) PBMC reference dataset to impute high-resolution cell clustering and surface protein expression and identify gene signatures predictive of survival. computing a gene expression score from the expression level of the at least two genes;

Therapeutic Methods

Also provided is a process of treating, preventing, or reversing COVID-19 in a subject in need of administration of a therapeutically effective amount of a COVID-19 treatment. The methods described herein may be used to identify subjects as having or likely to develop critical COVID-19 and/or discriminate said subjects from those having mild COVID-19 or unlikely to develop critical COVID-19. As some therapeutics are specifically indicated for critical COVID-19 or prevention of COVID-19, the methods described herein may be used to select an appropriate course of treatment for the subject.

Methods described herein are generally performed on a subject in need thereof. A subject in need of the therapeutic methods described herein can be a subject having, diagnosed with, suspected of having, or at risk for developing COVID-19. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a COVID-19 treatment is, for example, an amount that would cause the desired therapeutic effect in a subject while minimizing undesired side effects. In various embodiments, an effective amount of a COVID-19 treatment described herein can substantially inhibit COVID-19 infection, slow the progress of COVID-19 infection, or limit the development of symptoms associated with COVID-19 infection.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a COVID-19 treatment can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat COVID-19 infection.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀ , (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a COVID-19 treatment can occur as a single event or over a time course of treatment. For example, a COVID-19 treatment can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for COVID-19.

A COVID-19 treatment can be administered simultaneously or sequentially with another agent, such as an antibiotic, an anti-inflammatory, or another agent. For example, a COVID-19 treatment can be administered simultaneously with another agent, such as an antibiotic or an anti-inflammatory. Simultaneous administration can occur through administration of separate compositions, each containing one or more of a COVID-19 treatment, an antibiotic, an anti-inflammatory, or another agent. Simultaneous administration can occur through administration of one composition containing two or more of a COVID-19 treatment, an antibiotic, an anti-inflammatory, or another agent. A COVID-19 treatment can be administered sequentially with an antibiotic, an anti-inflammatory, or another agent. For example, a COVID-19 treatment can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025); and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the COVID-19 treatment may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a COVID-19 treatment such as described herein may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

COVID-19 Treatments and Interventions

As described herein, patients infected with SARS-CoV-2 display a wide range of disease severity ranging from asymptomatic or mild infection to severe or critical illness with multiple organ failure. Critically ill cases of COVID-19 may present with respiratory and cardiac failure or require intensive care support, and often portend high mortality rates. Subjects having critical COVID-19 may also present with kidney failure, sepsis, thrombosis, shock, or acute respiratory distress syndrome (ARDS), and may require mechanical ventilation or intubation.

COVID-19 treatments and interventions in accordance with the present disclosure may include those used under the current standard of care or experimental treatments or interventions.

As described herein, some COVID-19 treatments may be specifically indicated for subjects having or at risk of developing severe or critical COVID-19. For example, the antiviral therapeutics Paxlovid, Lagevrio (molnupiravir), and Veklury (remdesivir), as well as the monoclonal antibody treatment bebtelovimab, have been authorized by the FDA to treat subjects having COVID-19 and at risk for severe or critical illness. Convalescent plasma and the immune modulators Olumiant (baricitinib) and Actemra (tocilizumab) have also been authorized for similar indications. In addition, current NIH clinical guidelines recommend the use of systemic corticosteroids such as dexamethasone, tofacitinib, or sarilumab for use in hospitalized subjects having or at risk of developing severe or critical COVID-19.

COVID-19 treatments for subjects who have mild COVID-19 and are unlikely to develop severe or critical COVID-19 typically comprise over-the-counter (OTC) medications aimed at symptom management. For example, such OTC medications may include fluids, pain relievers and/or fever reducers such as acetaminophen or ibuprofen, cough suppressants, or expectorants.

Experimental COVID-19 treatments may be, for example, treatments that are not yet FDA-approved or are currently in clinical trials. Because experimental treatments have not been determined as safe and effective by the FDA, there may be a risk of serious side effects; thus, such treatments are typically provided when a subject has a serious or life-threatening illness for which satisfactory treatments are not available (e.g., “compassionate use”). The present disclosure contemplates that the methods herein may be used to determine whether a subject qualifies for an experimental COVID-19 treatment. For example, a subject with critical COVID-19 who is found to be at high risk for mortality using the methods of the present disclosure, may be administered an experimental treatment alone or in addition to standard treatments. As another example, a subject with critical COVID-19 who is found to be at low risk for mortality may not be administered an experimental treatment.

Examples of experimental treatments for COVID-19 currently in clinical trials include hydroxychloroquine, vitamin C, GC4419, Cannabidiol, Mesenchymal Stem Cells Transplantation, Ketogenic diet, Leronlimab, FP-025, Apixaban, N-acetylcysteine, Crizanlizumab, Sodium Thiosulfate, ALLOCETRA-OTS, Aviptadil, Remimazolam, Auxora, Bevacizumab, Lenzilumab, Tradipitant, Imatinib, MRG-001, Sirukumab, and Dapsone.

In some embodiments, a subject found to be at high risk of mortality using the methods described herein may receive the same COVID-19 treatment as a subject that is found to be at low risk of mortality, but at a higher dose or for a longer duration.

In some embodiments, a subject found to be at high risk of mortality using the methods described herein may be prioritized for treatment, intensive care unit (ICU) admission, ventilator use, or hospitalization over a subject that is found to be at low risk of mortality, particularly in a situation where healthcare resources are limited or scarce.

Methods For Monitoring COVID-19

The present disclosure provides a method for monitoring critical COVID-19 in a subject. In such an embodiment, a first expression level of at least two genes found to be predictive of COVID-19 mortality (see e.g., Example 1) is measured and a first gene expression score computed, which may be used to assess the risk of a subject at one point in time. Then at a later time, a second expression level of the at least two genes is measured and a second gene expression score is computed, which may be used to determine the change in risk of the subject over time. For example, such a method of monitoring may be used on the same subject days, weeks, or months following the initial measurement of a first expression level and computing a first gene expression score. The change in gene expression score, as computed by the difference between the first expression score and the second expression score, indicates a change in the subject's risk of mortality from critical COVID-19. In some embodiments, a change in the subject's risk of mortality may be determined based on whether the second gene expression level is significantly different than the first gene expression level. The term ‘significantly different’ as used herein refers to a difference that is statistically significant, as measured by a suitable statistical test. In some embodiments, In exemplary embodiments, whether a second gene expression score is significantly different from a first gene expression score may be determined using a p-value. For example, when using a p-value, a second gene expression score is identified as being significantly different from a first gene expression score when the p-value is less than 0.1, less than 0.05, less than 0.01, less than 0.005, or less than 0.001. Administration of treatment for COVID-19 may be based at least in part on the change in gene expression score and/or on the change in the subject's change in risk of mortality.

In some embodiments, the method for monitoring COVID-19 may be used to measure the rate of disease progression. Accordingly, changes in a subject's risk of mortality as determined by the methods of the present disclosure may indicate disease progression or disease abatement, which may inform treatment decisions. As an example, a subject may initially be hospitalized with critical COVID-19 and a first gene expression score computed that indicates low risk of mortality. If a second gene expression score computed at a later time for the subject indicates a high risk of mortality, a clinician may decide to implement changes in a treatment plan for the subject; for example, additional or more aggressive COVID-19 treatments may be administered, such as mechanical ventilation or experimental therapies, or dosage or duration of a treatment may be increased. If the second gene expression score continues to indicate a low risk of mortality, a clinician may decide to make no changes to the treatment plan for the subject or to reduce dosage or duration of a treatment.

Likewise, the method for monitoring COVID-19 in a subject may also be used to determine the response to COVID-19 treatment, such as those described herein. In such an embodiment, a first expression level of at least two genes found to be predictive of COVID-19 mortality is measured and a first gene expression score computed from the biological sample of the subject prior to initiation of treatment. Then at a later time, a second expression level of the at least two genes is measured and a second gene expression score is computed, which may be used to determine the response to treatment over time. For example, if the subject's risk of mortality as determined by the methods of the present disclosure decreases or stays the same, then the subject may be responding to treatment. If the subject's risk of mortality increases, then the subject may not be responding to treatment. This method may be repeated over time to continually determine the subject's response to therapy.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to sequencing or assay reagents, software, or sequencer. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, a subject having survived critical COVID-19 or sample, or from populations thereof. A reference value can be used in place of a control or reference sample, which was previously obtained from a healthy subject or a group of healthy subjects or a wild-type subject or sample. Or a subject having survived critical COVID-19 or sample. A control sample or a reference sample can also be a sample with a known amount of a detectable compound or a spiked sample.

A biological sample as described herein refers to a sample obtained from a subject. The biological sample may be derived from any sample containing immune cells. Suitable biological samples may include bodily fluids, such as blood, plasma, serum, urine, and saliva. In a preferred embodiment, the biological sample is a blood sample. Suitable biological samples may also include tissue samples such as a tissue biopsy.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Cell Specific Peripheral Immune Responses Predict Survival in Critical COVID-19 Patients

The following example describes the prediction of survival in COVID-19 patients.

Summary

SARS-CoV-2 triggers a complex systemic immune response in circulating blood mononuclear cells. The relationship between immune cell activation of the peripheral compartment and survival in critical COVID-19 remains to be established. Herein is described the use of single-cell RNA sequencing and Cellular Indexing of Transcriptomes and Epitomes by sequence mapping to elucidate cell type specific transcriptional signatures (e.g., gene expression scores calculated from measured expression levels of at least two genes) that associate with and predict survival in critical COVID-19.

Patients who survive infection display activation of antibody processing, early activation response, and cell cycle regulation pathways most prominent within B-, T-, and NK-cell subsets. Cell specific differential gene expression and machine learning were further leveraged to predict mortality using single cell transcriptomes. Interferon signaling and antigen presentation pathways within cDC2 cells, CD14 monocytes, and CD16 monocytes were identified as predictors of mortality with 90% accuracy. Finally, the findings were validated in an independent transcriptomics dataset and a framework to elucidate mechanisms that promote survival in critically ill COVID-19 patients was provided. Identifying prognostic indicators among critical COVID-19 patients holds tremendous value in risk stratification and clinical management.

Introduction

Severe acute respiratory syndrome coronavirus 2 (SARS-CoV-2) is the pathogenic agent responsible for the novel coronavirus disease (COVID-19), which has led to a global pandemic with over 275 million cases and >5.3 million deaths as of December 2021. Patients infected with SARS-CoV-2 display a wide range of disease severity ranging from asymptomatic or mild infection to critical illness with multiple organ failure. Critically ill cases of COVID-19 present with respiratory and cardiac failure, require intensive care support, and portend high mortality rates.

While prior studies have utilized single-cell omics to unravel the immunological landscape of COVID-19 in peripheral blood mononuclear cells (PBMCs), there remains an incomplete understanding of the relationship between peripheral immune cell activation and patient survival. Current cross-sectional studies have yet to identify immune cell types and transcriptional programs that contribute to survival in critical COVID-19. This information is necessary to effectively develop strategies to treat the sickest COVID-19 patients. Herein is described the performance of single-cell RNA sequencing (scRNA-seq) of PBMCs from patients with critical COVID-19 who survived (n=6) or died (n=6) at both days 0 and 7 of study enrollment with associated age-matched controls (n=6). To obviate sparsity concerns associated with scRNA-seq cluster annotations, the dataset was mapped onto a Cellular Indexing of Transcriptomes and Epitomes by sequencing (CITE-seq) PBMC reference dataset to impute high-resolution cell clustering and surface protein expression.

Patients who survive COVID-19 were found to exhibit signatures associated with humoral immunity including B-cell activation, cell cycle regulation, and plasmablast antibody processing on day 7. A negative association was also uncovered between survival and increased interferon signaling in naive B-cells, naive CD8 T-cells, NK cells, and MAIT cells at this timepoint. To predict survival based on the earlier timepoint of enrollment, a random forest classifier machine learning model was utilized. CD14 monocytes, CD16 monocytes, and type II conventional dendritic cells (cDC2s) were identified as predictors of mortality on day 0. Interferon stimulated genes (ISGs) such as IFITM1, IFITM3, and IFI27 were among the strongest early prognostic features. Through an integrated approach consisting of differential gene expression analysis and gene ranking by feature importance score, it was further shown that CEBPD, MAFB, IFITM3, and LGALS1 expression within CD14 monocytes robustly predict mortality. The framework was validated and the genetic signature refined in an independent dataset from Liu et al., which supports specific enriched expression among survivors at day 0. Together, these findings provide a framework to elucidate mechanisms that promote COVID-19 survival among critically ill patients and delineate key cell-specific transcriptional signatures that are associated with mortality in critical COVID-19.

These transcriptional signatures, which in some embodiments comprise gene expression scores calculated from measured expression levels of at least two genes, are associated with and predict survival in critical COVID-19. According to the present disclosure, gene expression levels from at least two genes are measured from a biological sample and are used to compute a gene expression score. In exemplary embodiments, the gene expression levels are measured from immune cells present in a peripheral blood sample of a human subject. By comparing the gene expression score to a reference score computed from a reference sample, the subject is determined to have a high risk of mortality from critical COVID-19 if the gene expression score is significantly different from the reference score. Alternately, the subject is determined to have a low risk of mortality from critical COVID-19 if the gene expression score is not significantly different from the reference score. The term ‘significantly different’ as used herein refers to a difference that is statistically significant, as measured by a suitable statistical test. In exemplary embodiments, whether a gene expression score is significantly different from a reference score may be determined using a p-value. For example, when using a p-value, a gene expression score is identified as being significantly different from the reference score when the p-value is less than 0.1, less than 0.05, less than 0.01, less than 0.005, or less than 0.001.

As disclosed herein, the determination of high or low risk of mortality for a subject informs subsequent treatment for the subject/patient. In some embodiments, where several COVID-19 subjects are present (e.g., several critical COVID-19 patients), the gene expression score for each patient serves to both inform as well as prioritize administration of treatment for each patient. For example, among patients determined to have high risk of mortality, various treatment(s) may be prioritized to the patient(s) having a gene expression score furthest/having the most significant difference from the reference score.

Results

Single-Cell RNA Sequencing Reveals the Landscape of PBMCs During The Evolution of Critical COVID-19.

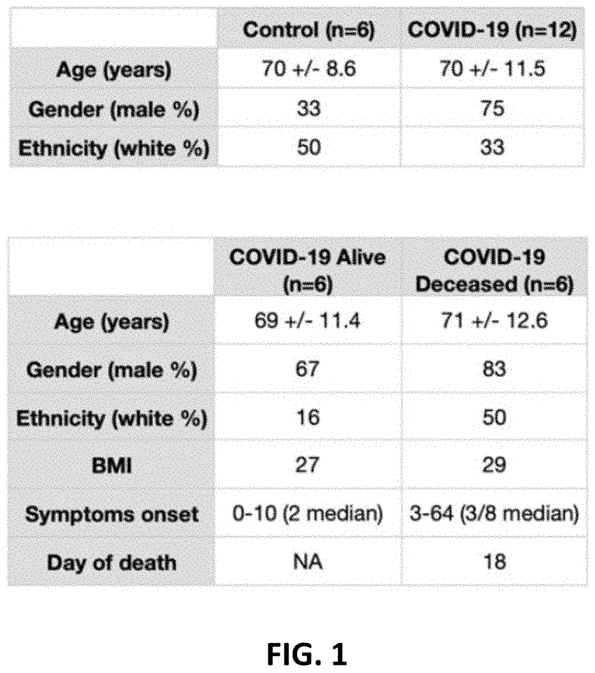

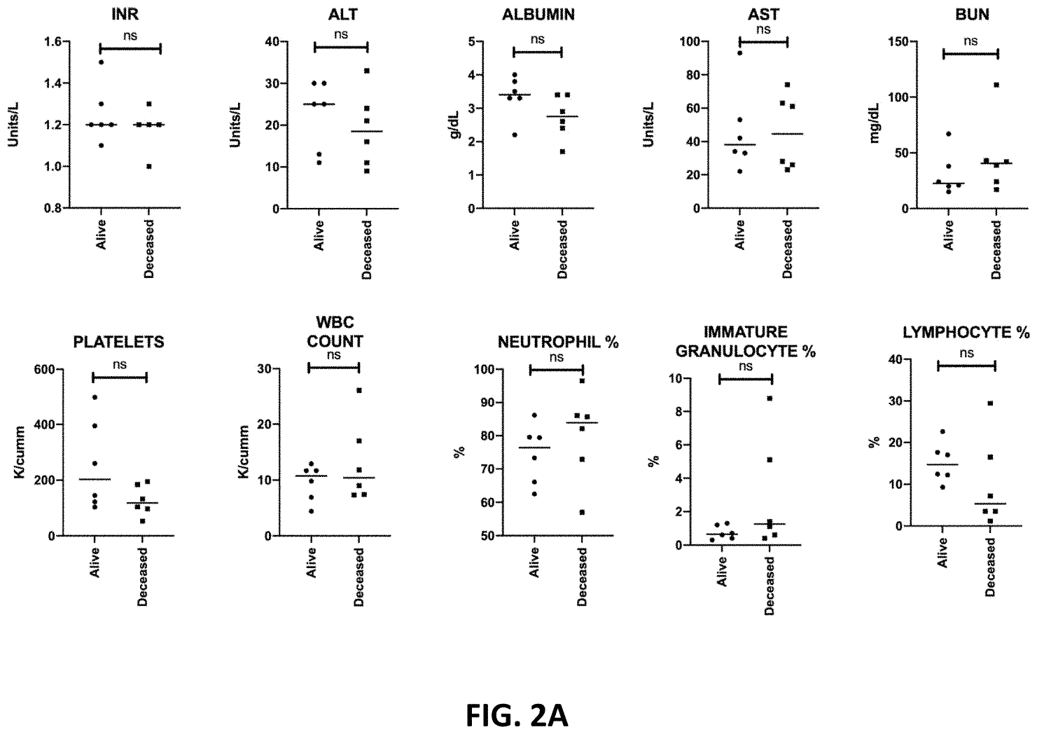

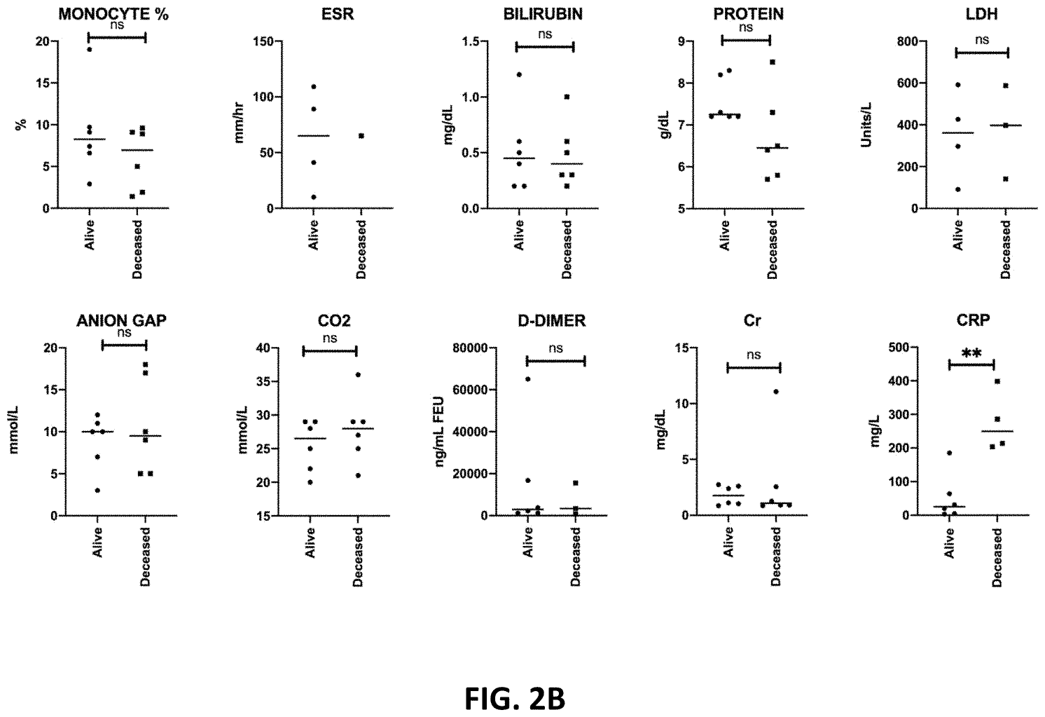

Patients were selected from approximately 500 subjects enrolled in Washington University's COVID-19 WU350 study. Those with critical COVID-19 defined by the requirement for admission to the intensive care unit were included. Twelve patients were chosen and further divided into those who survived infection (n=6) and those who succumbed to infection (n=6), all of whom had PBMCs banked at days 0 and 7 of study enrollment. PBMCs were collected from age- and sex-matched healthy controls (n=6). Patient clinical characteristics were similar between controls and those with critical COVID-19 and between those who survived and succumbed to COVID-19 (see e.g., FIG. 1). Routine medical laboratory testing only noted an increase in C-reactive protein in the deceased cohort (see e.g., FIG. 2A-FIG. 2B).

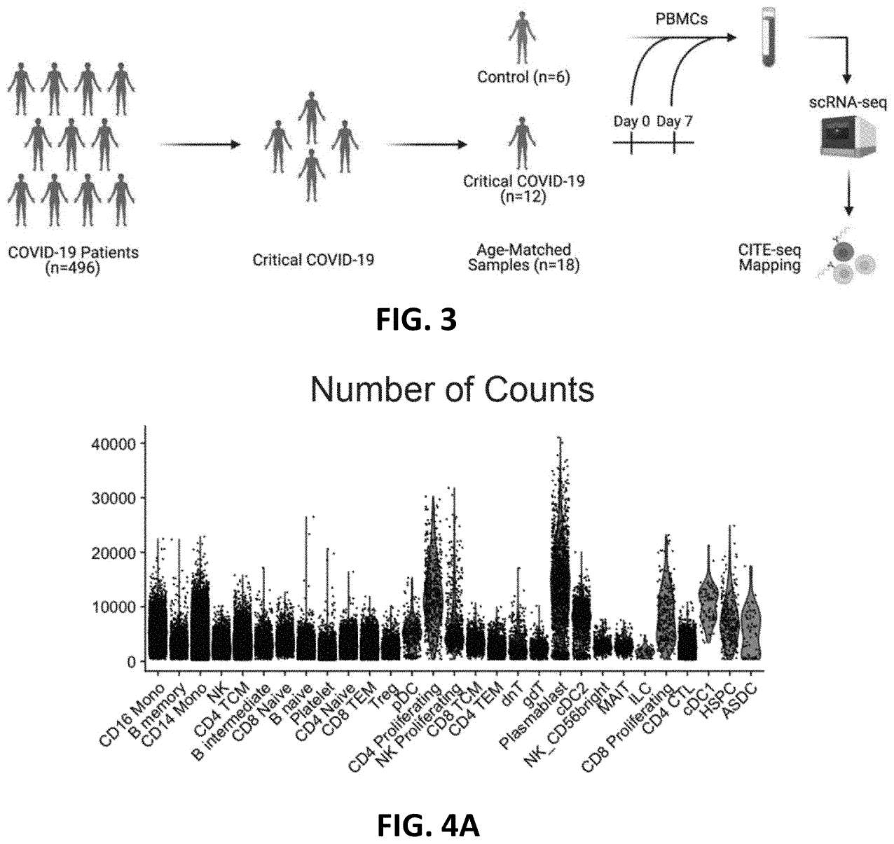

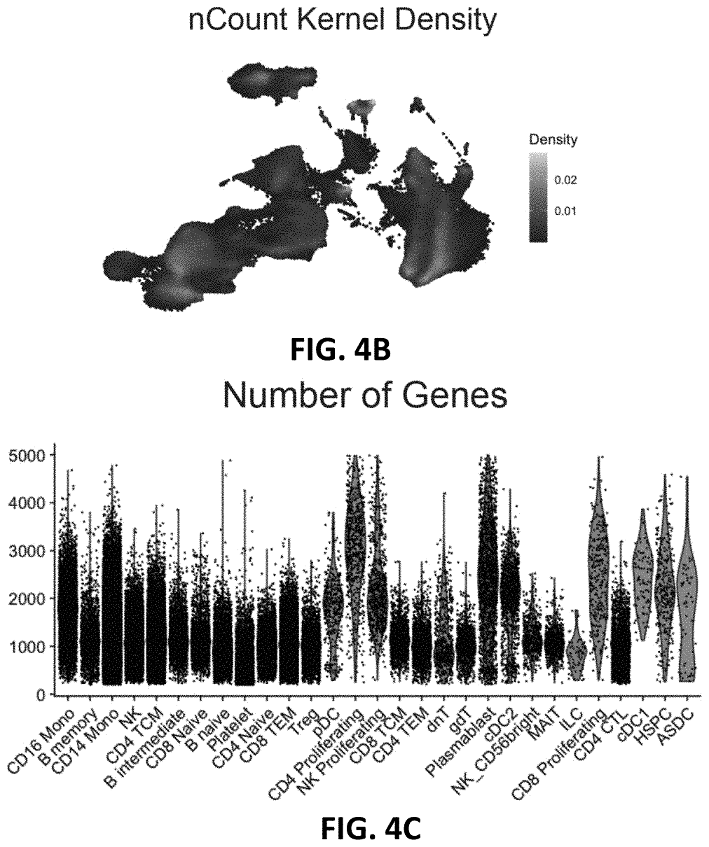

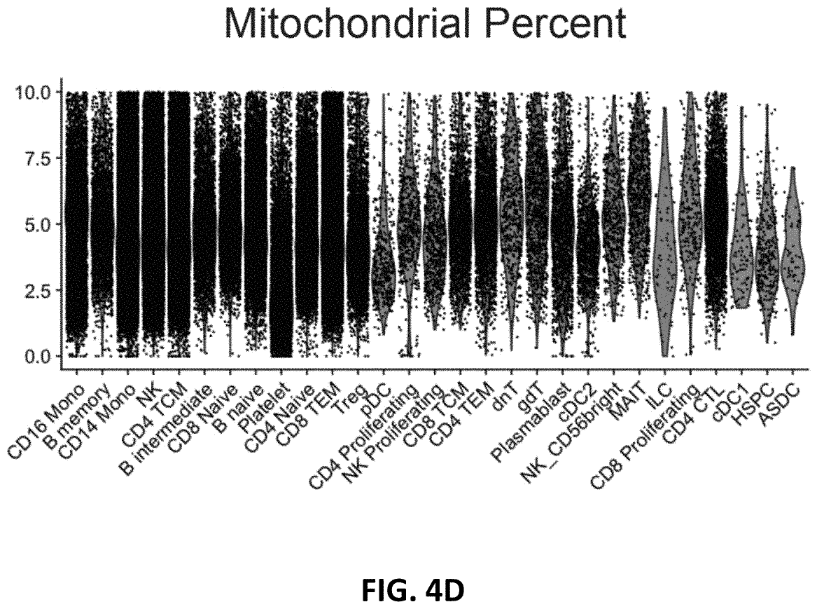

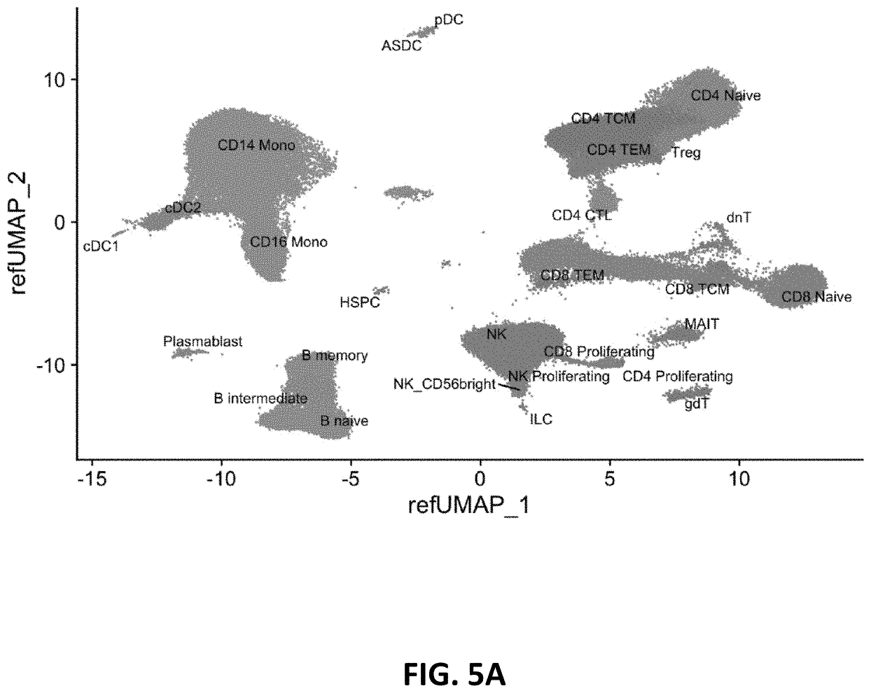

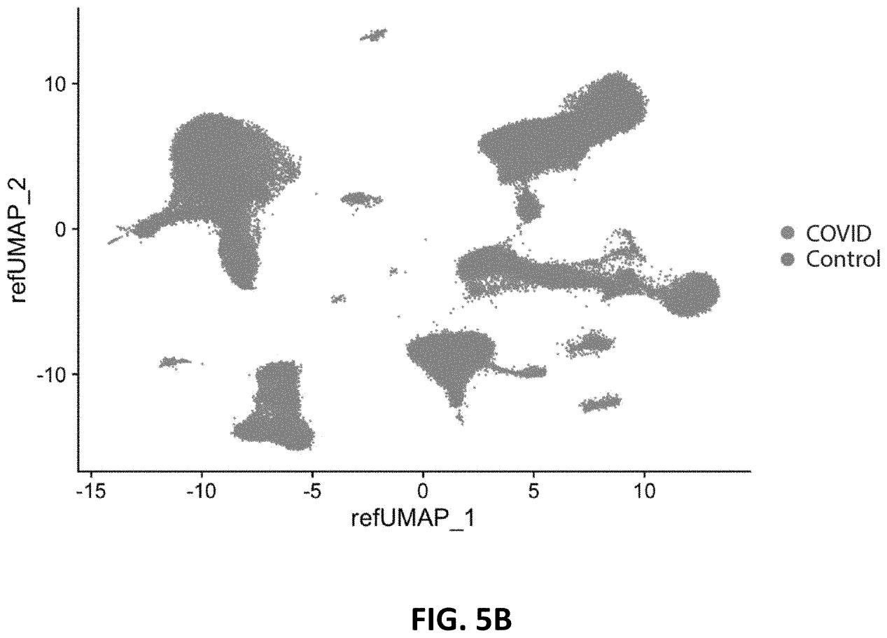

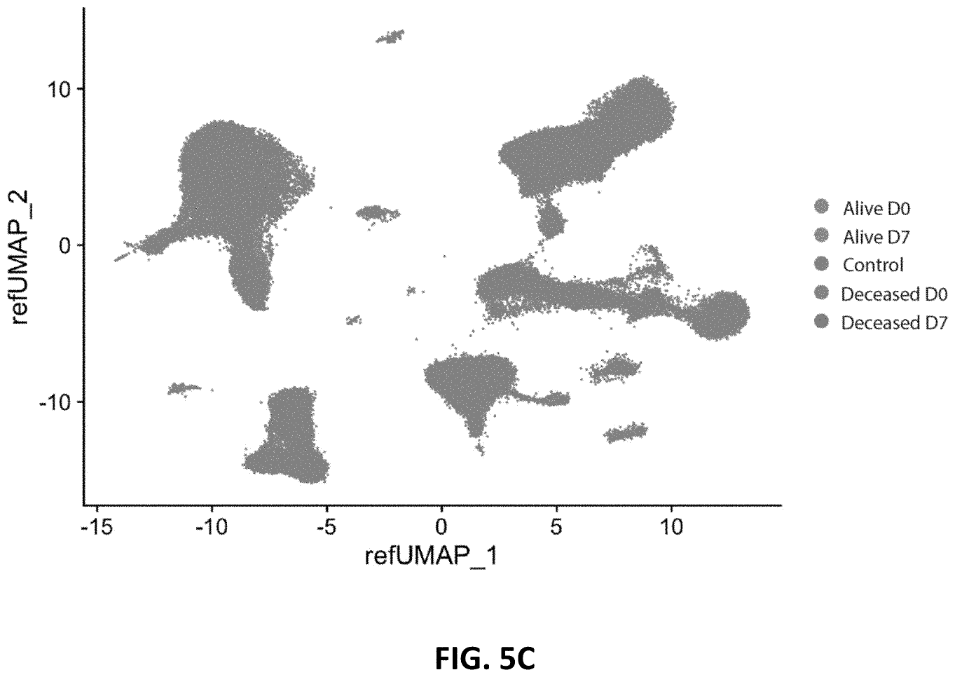

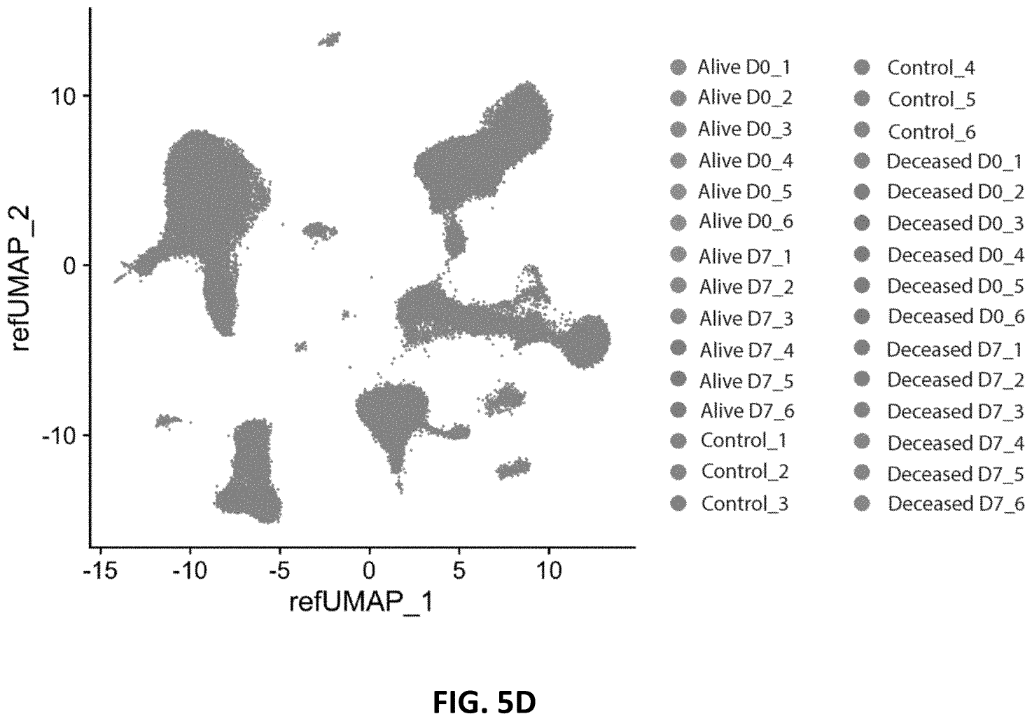

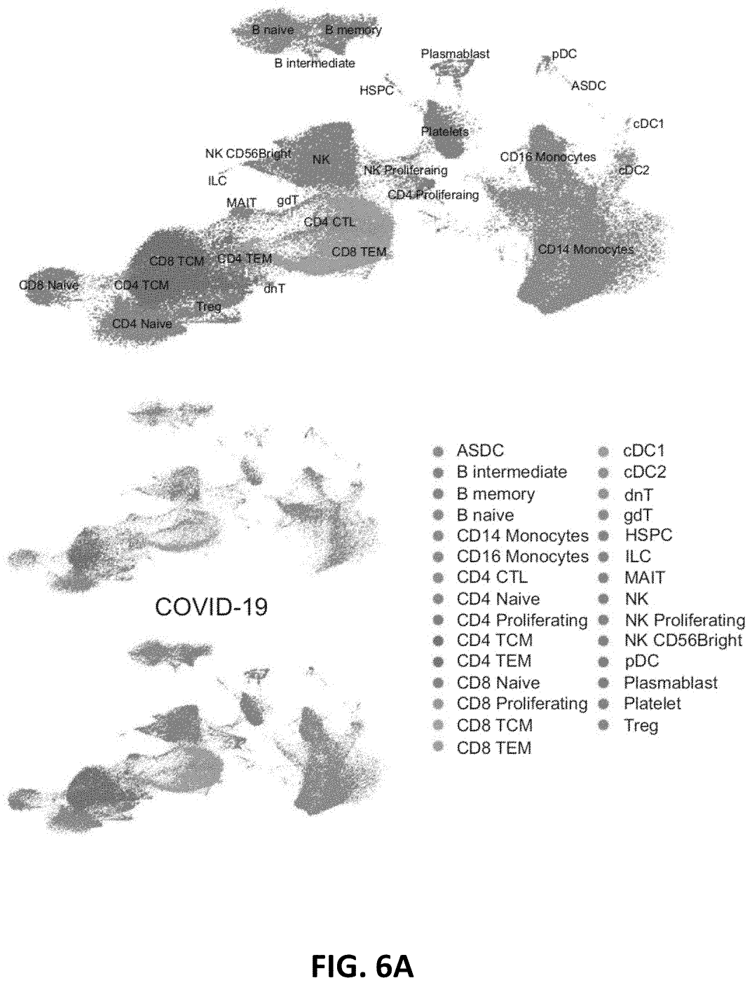

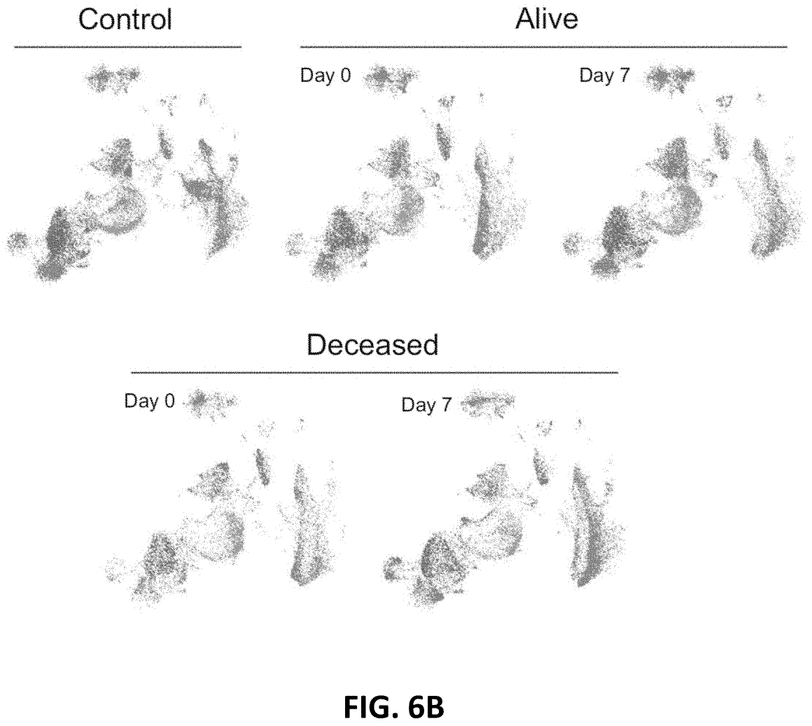

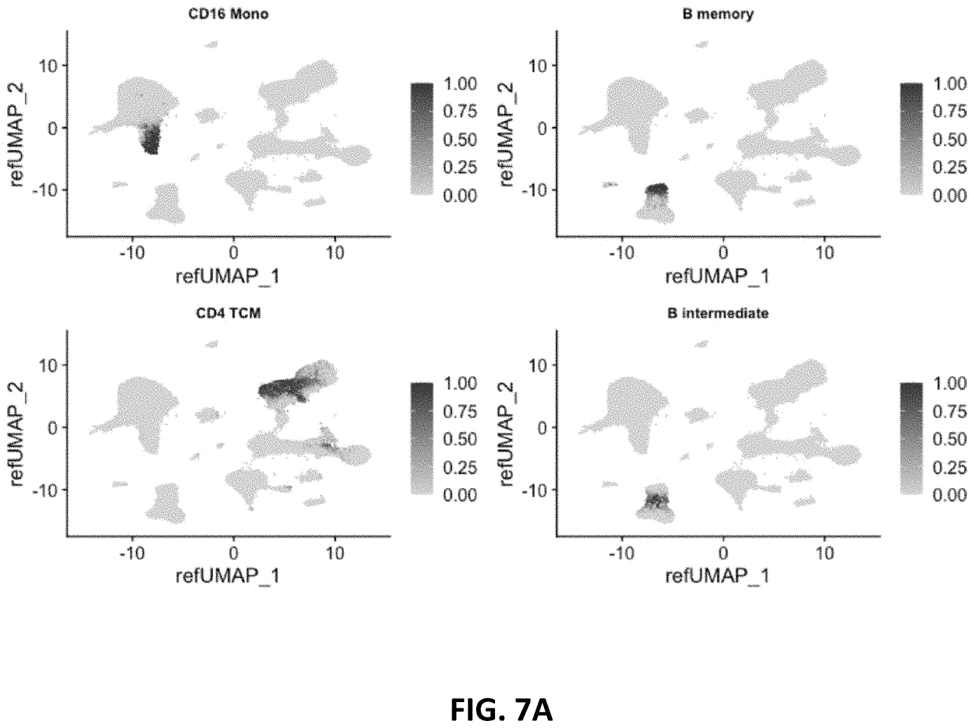

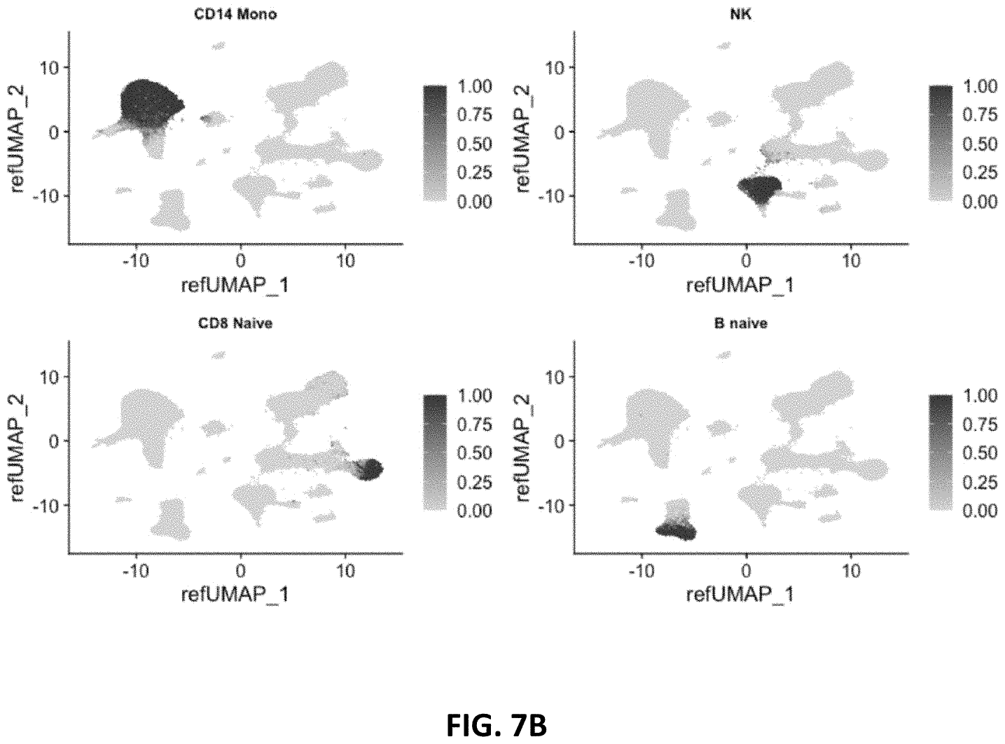

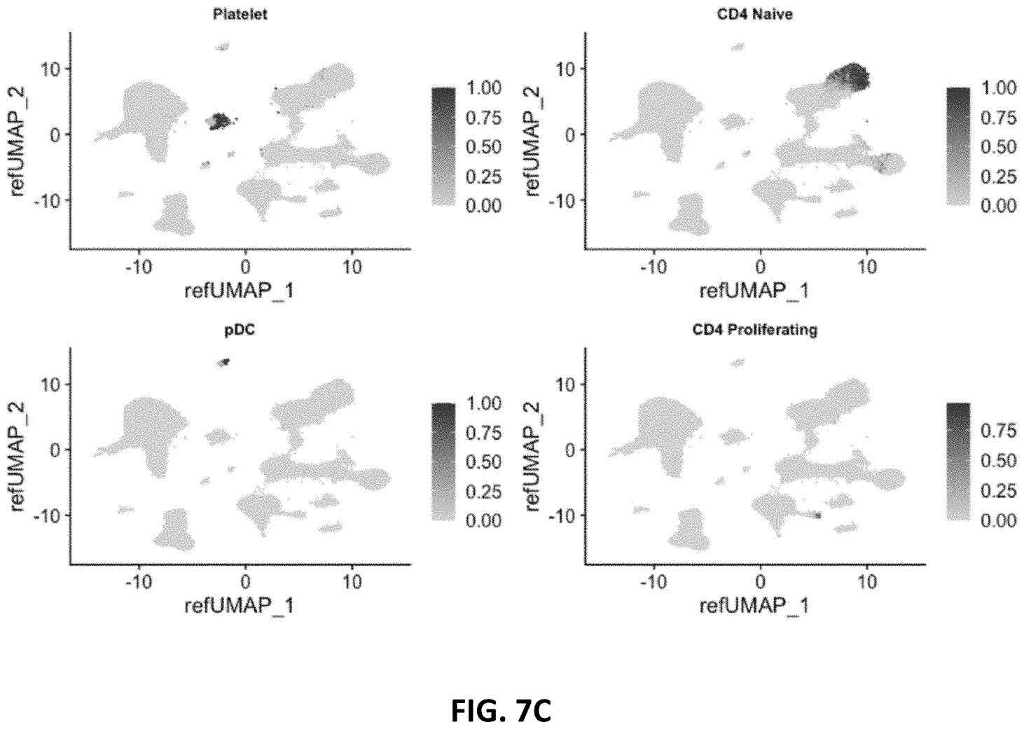

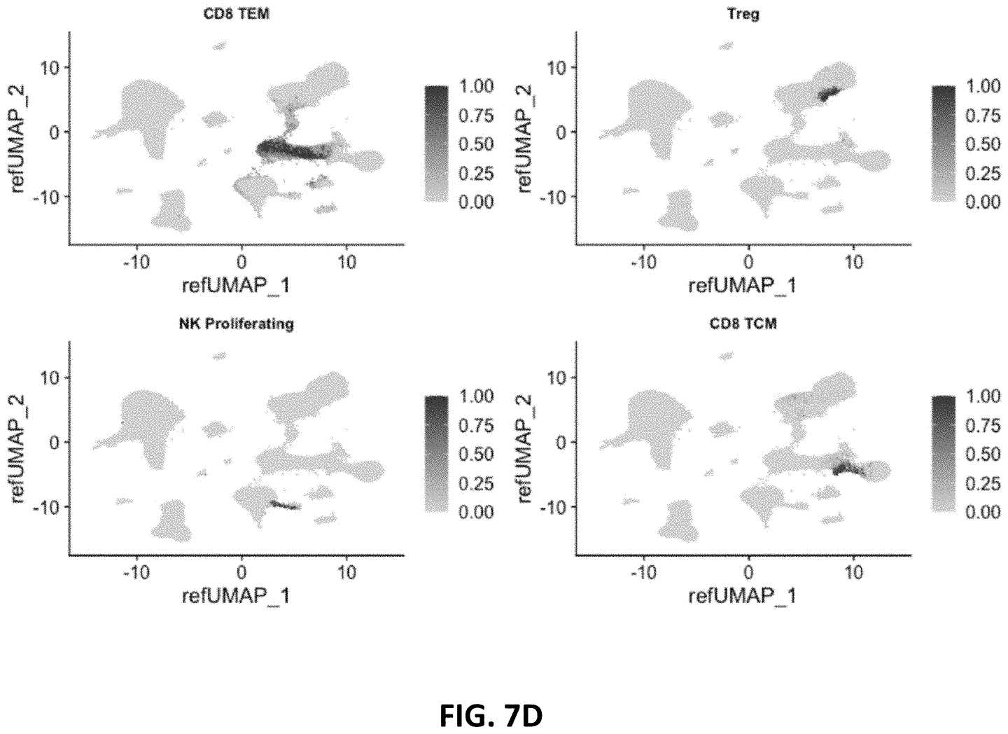

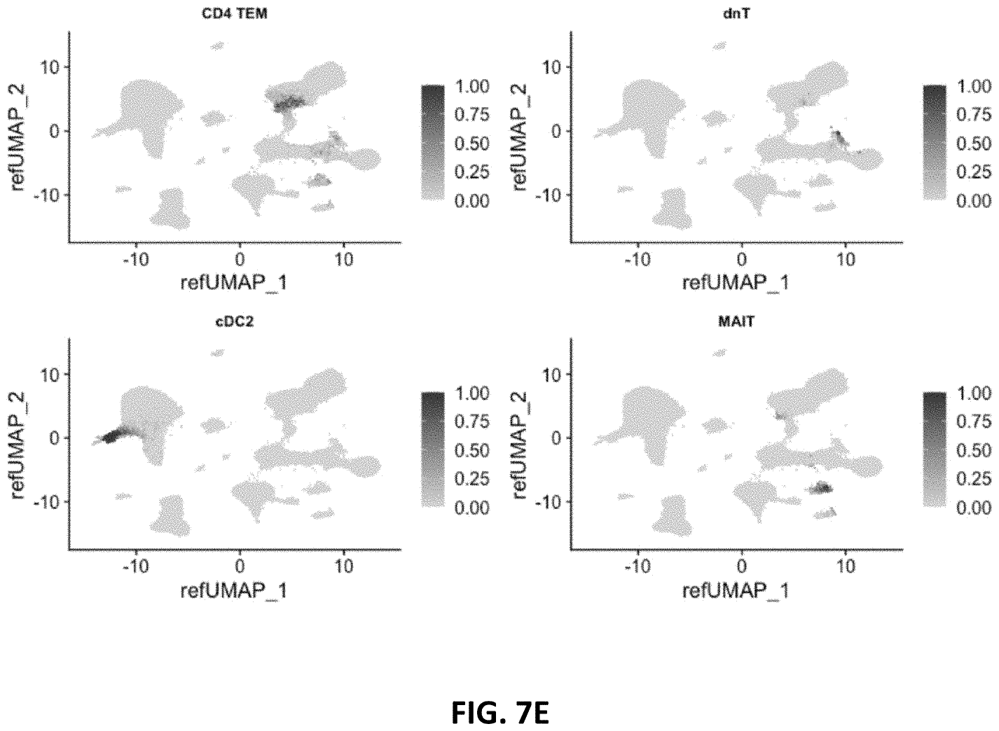

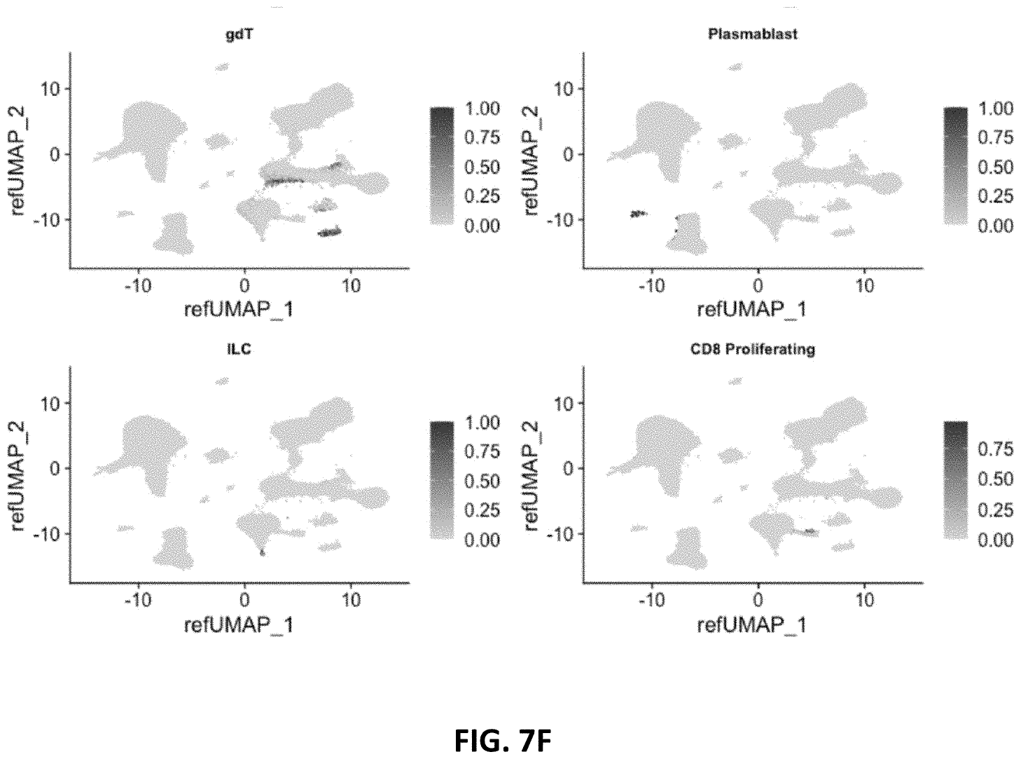

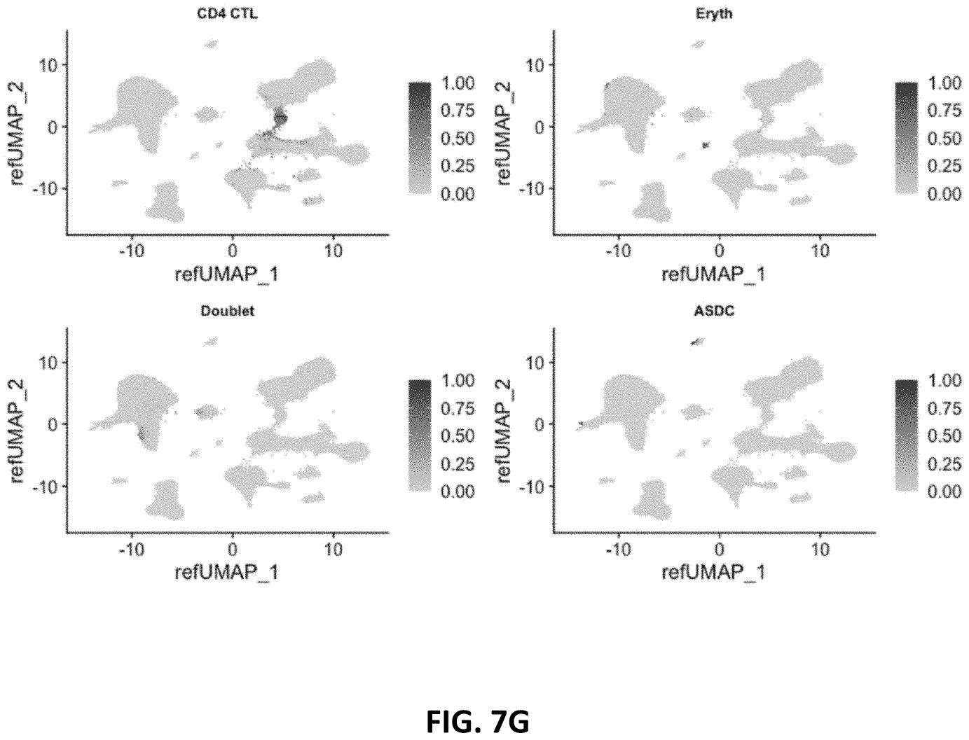

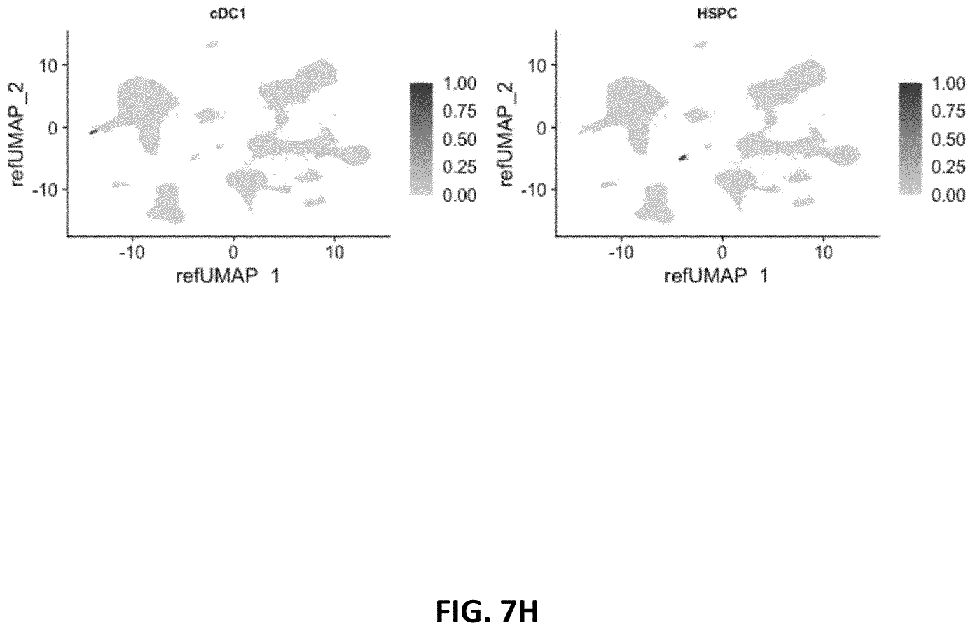

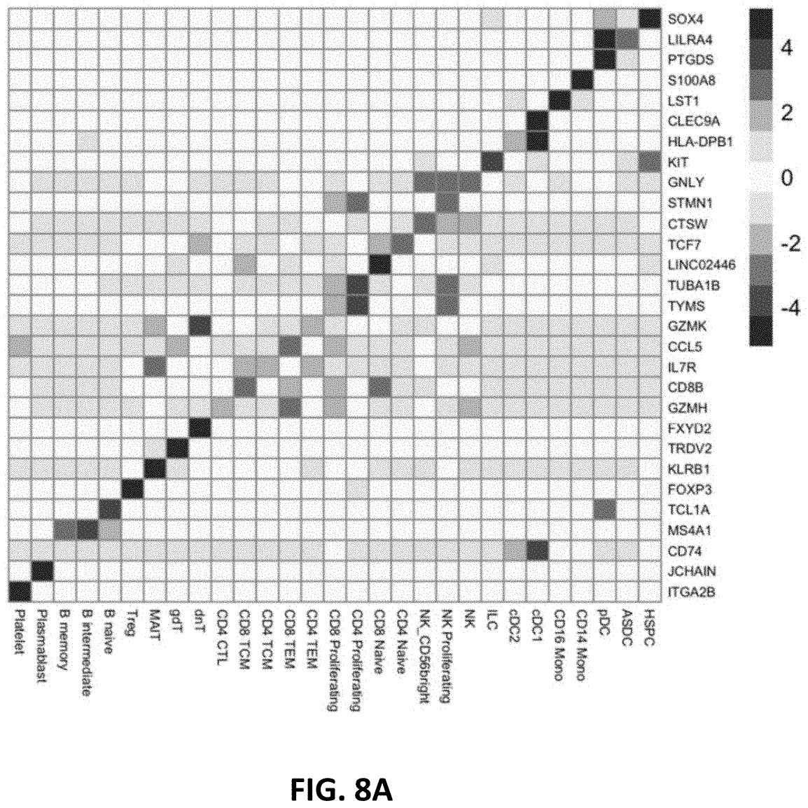

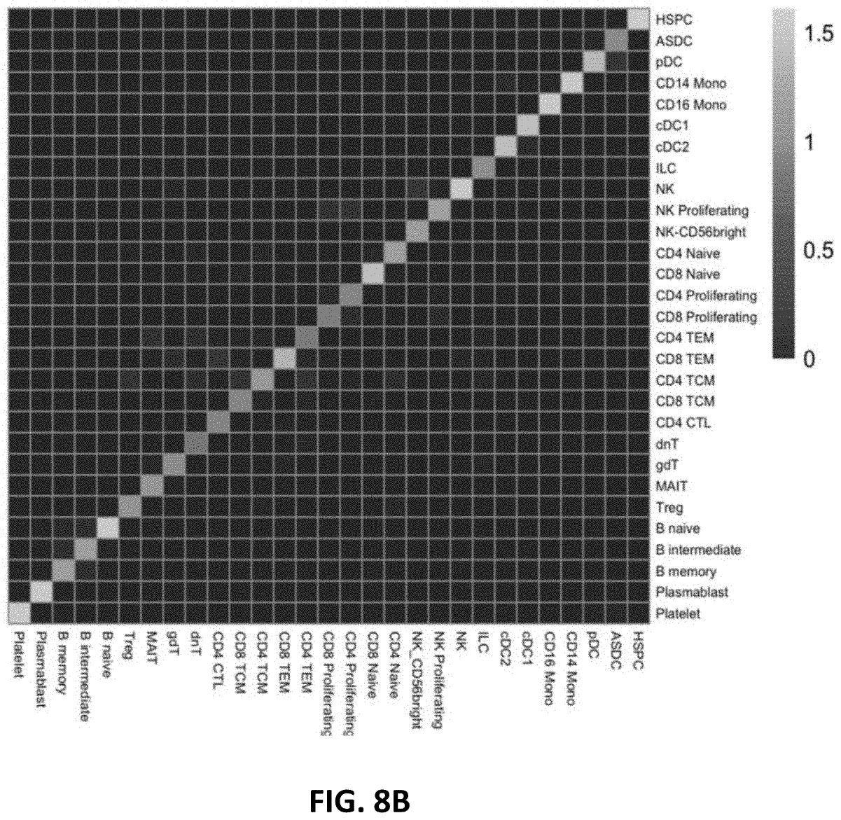

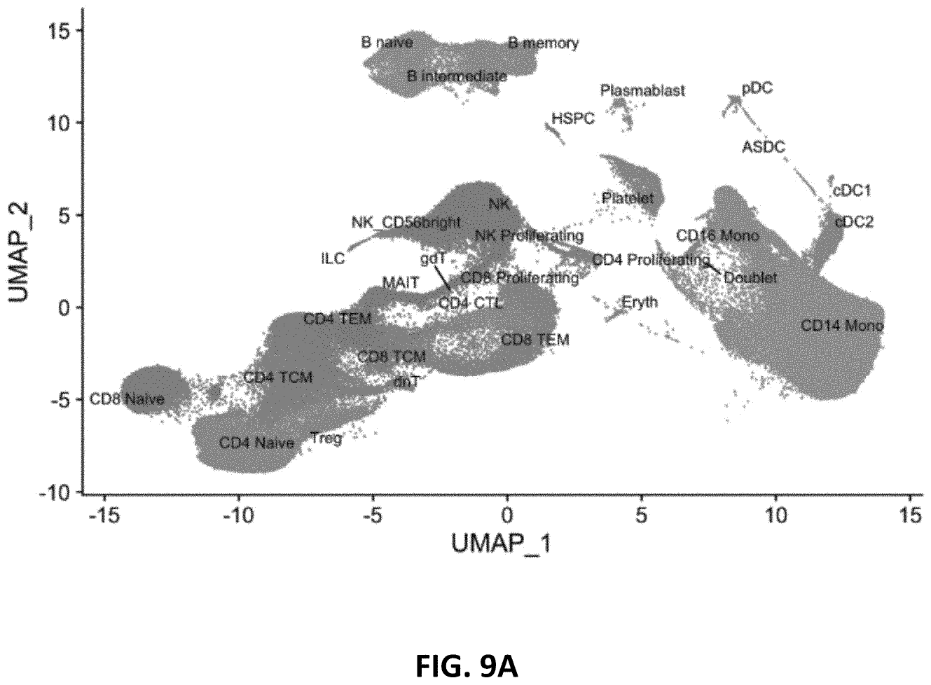

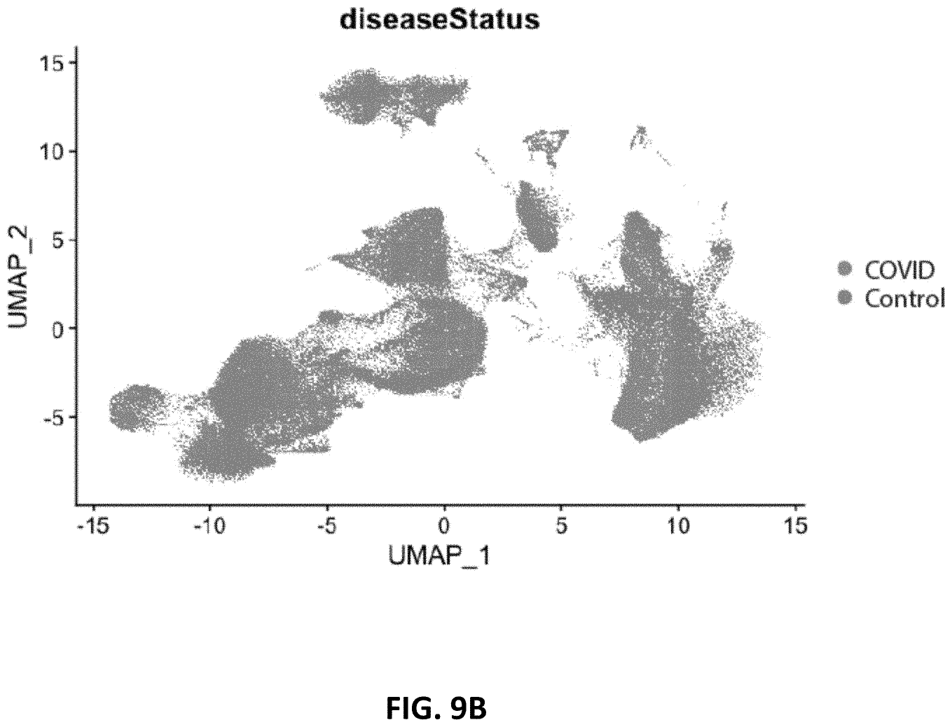

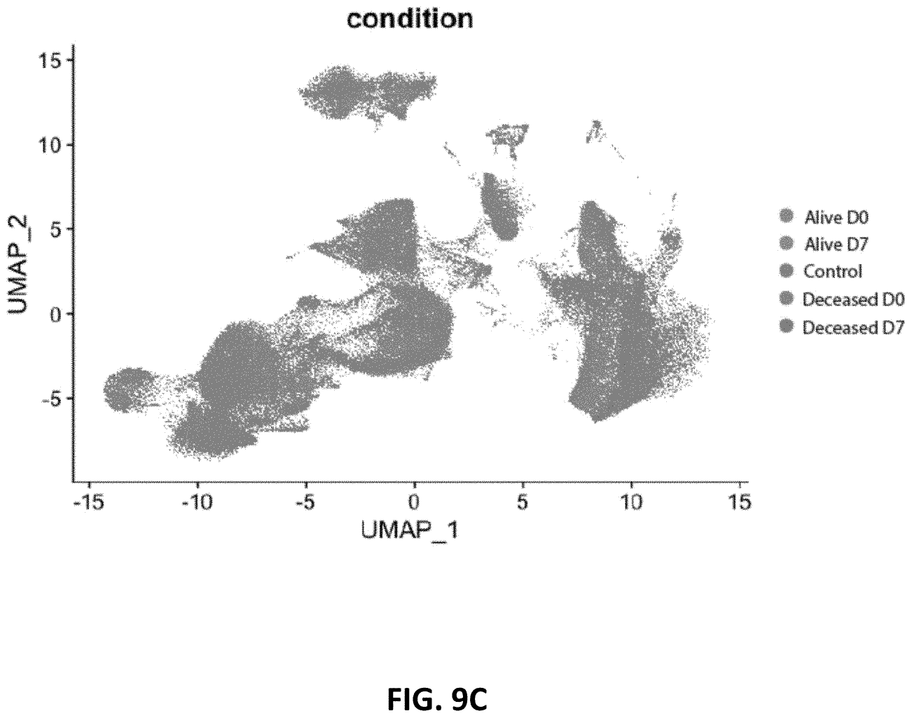

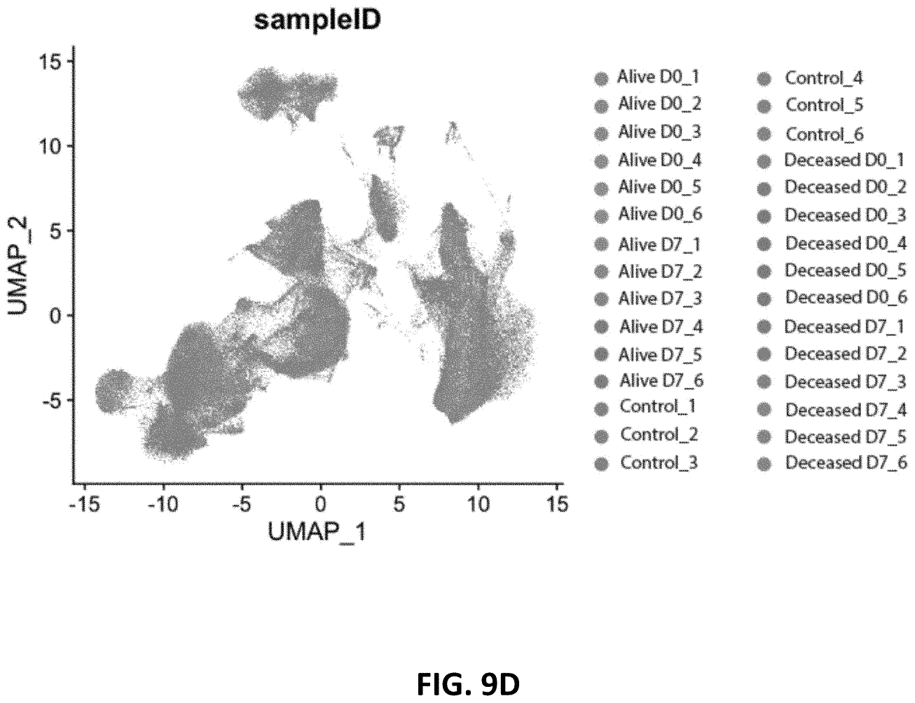

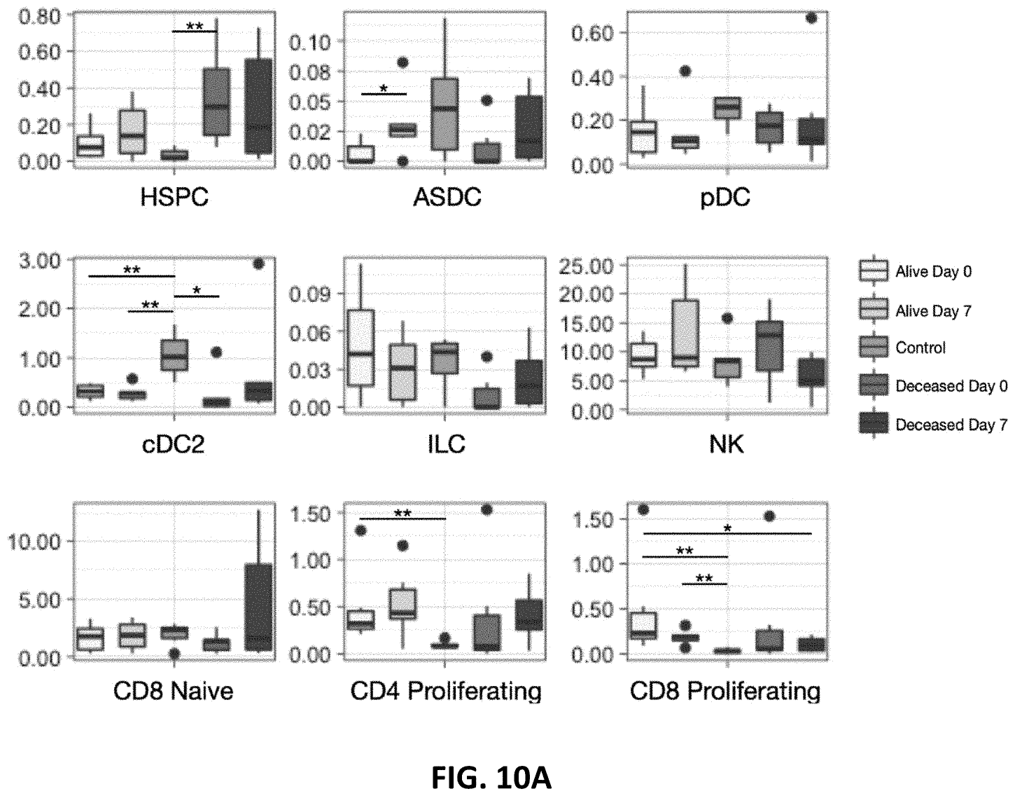

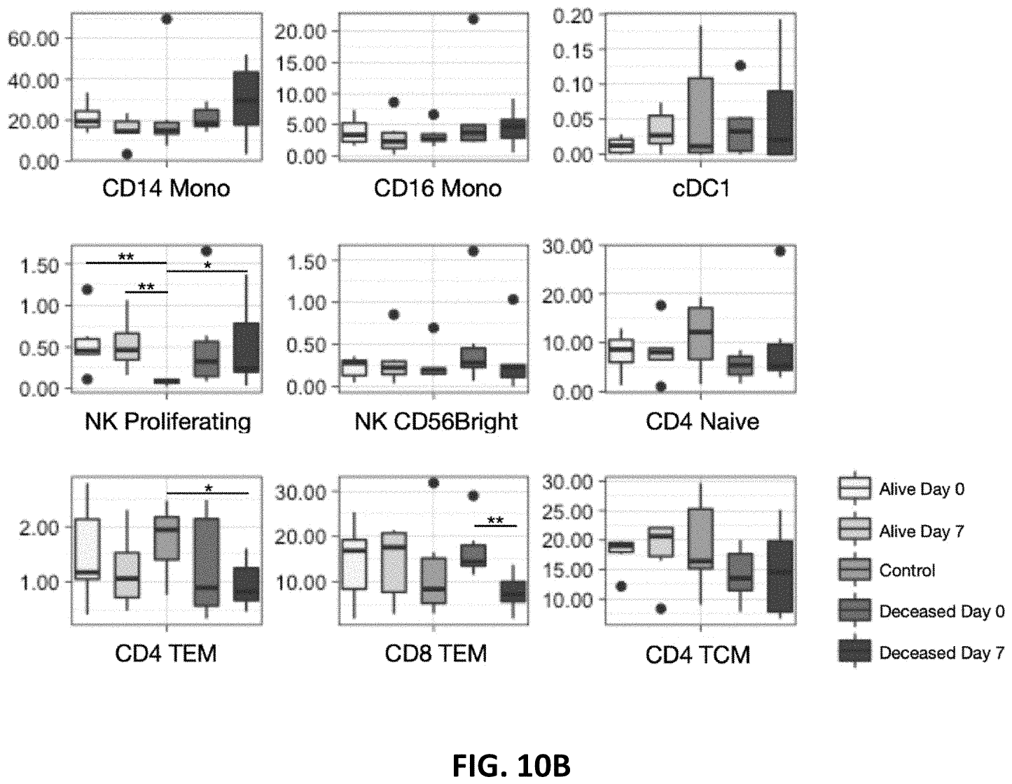

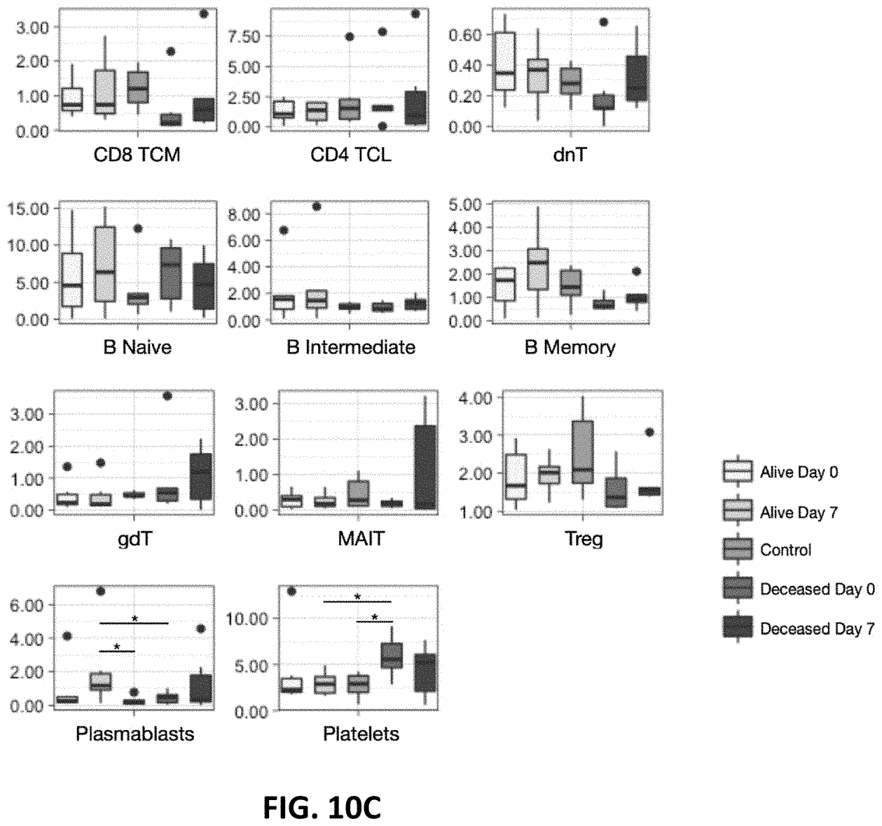

To profile the immune landscape, droplet based scRNA-seq was performed on PBMCs extracted from 30 samples and 199,097 cells expressing 24,675 genes were analyzed after applying quality control filters (see e.g., FIG. 3 and FIG. 4A-FIG. 4D). To delineate key immune cell subtypes, the dataset was mapped onto a publicly available CITE-seq PBMC reference (azimuth) and surface-protein expression was imputed for 228 markers allowing high-resolution cell identification with resultant annotation of 29 distinct clusters across conditions (see e.g., FIG. 5A-FIG. 5D). De novo UMAP visualizations were then computed to separate unique cell states in the data not included in the reference (see e.g., FIG. 6A-FIG. 6B and FIG. 7A-FIG. 7H). This mapping identified cells with well-defined canonical gene markers (see e.g., FIG. 8A) and high prediction accuracy (see e.g., FIG. 8B and FIG. 9A-FIG. 9D) through differential gene expression (DGE) testing. The relative proportion of each cell type was calculated and an expansion of progenitor cells, proliferating NK cells, T-cell subsets, plasmablasts and platelets but a reduction in cDC2 cells among critical COVID-19 patients was found (see e.g., FIG. 10A-FIG. 10C).

Distinct Immune Cell Populations Are Associated With Critical COVID-19 and Survival.

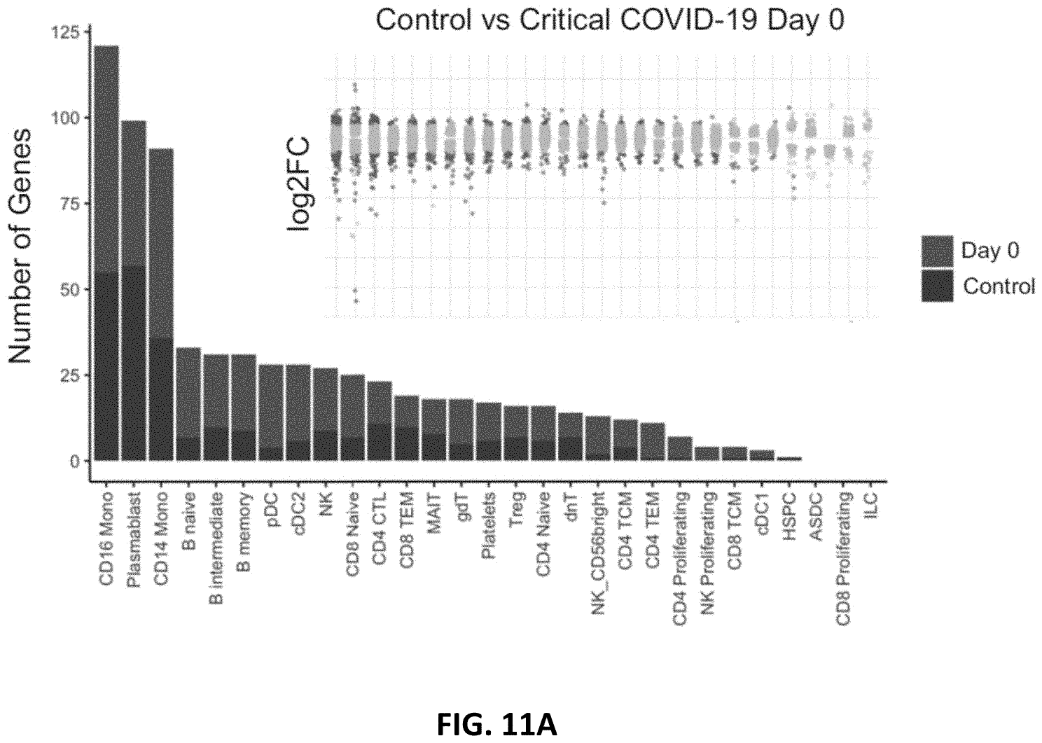

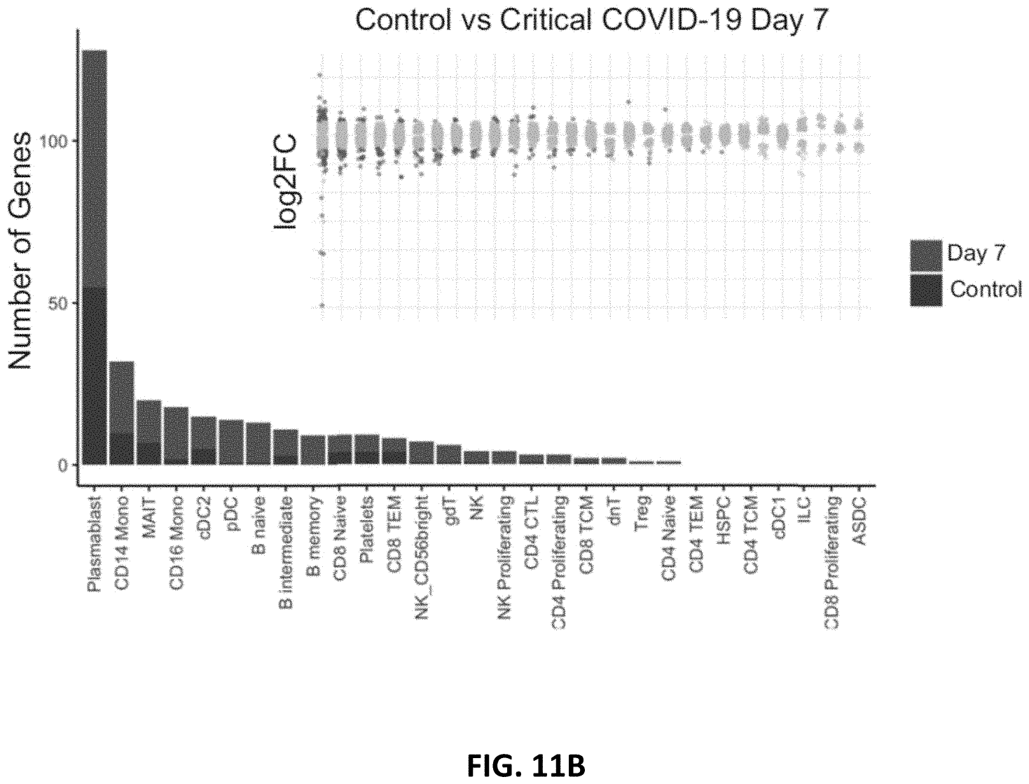

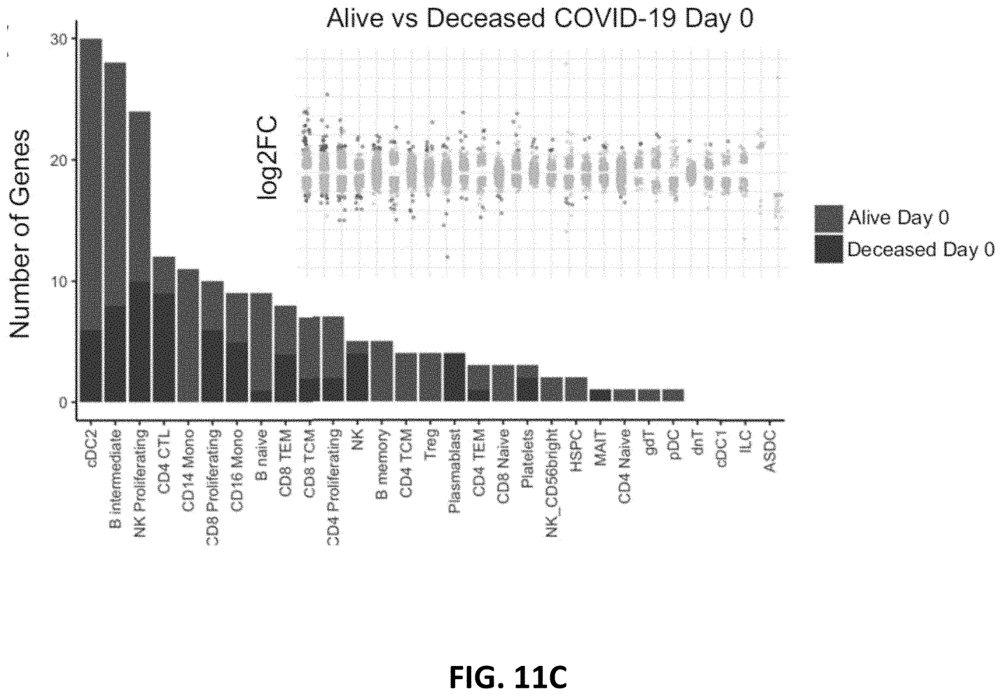

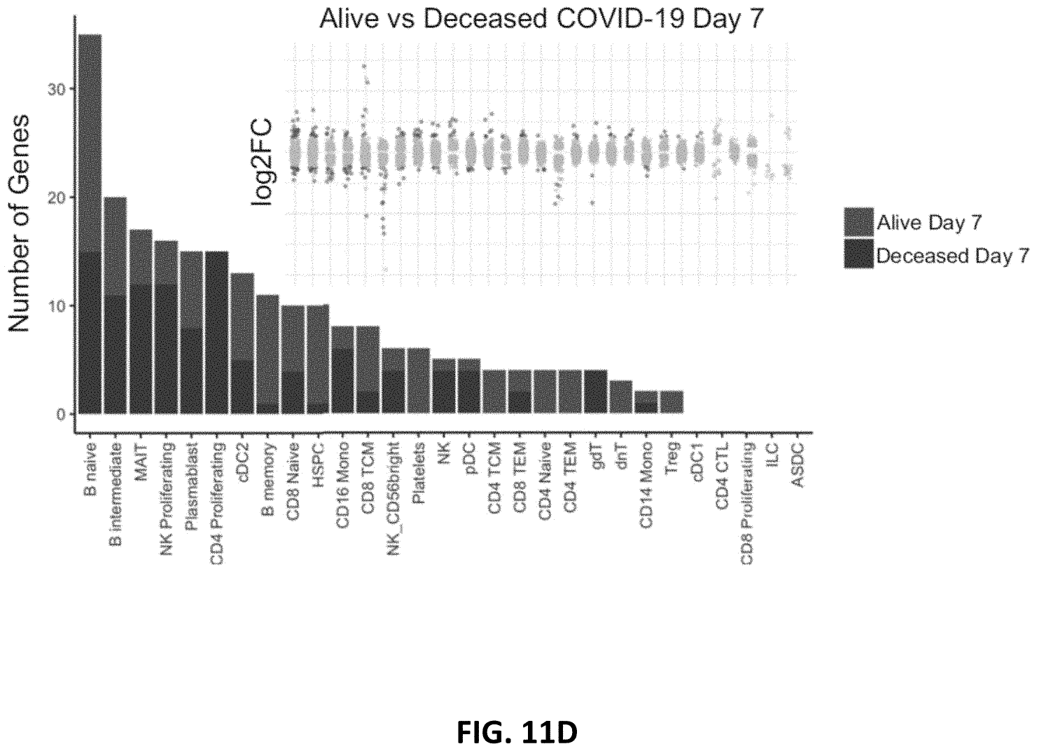

Cell state-specific DGE testing between key conditions was performed to identify individual cell populations associated with disease and survival at each examined timepoint. Consistent with prior reports comparing healthy controls and COVID-19 patients, strong transcriptional signatures associated with disease was observed in monocytes, plasmablasts, and B-cell subsets on day 0 and in plasmablasts, monocytes, and cDCs on day 7 (see e.g., FIG. 11A-FIG. 11B). Next, to identify immune cell types and corresponding transcriptional programs associated with survival, differential gene expression analysis was performed between samples from the alive and deceased cohorts at both days 0 and 7. cDC2 cells, CD14 monocytes, CD16 monocytes, NK-cells, naive and intermediate B-cells, and select T-cell subsets displayed transcriptional signatures associated with survival on day 0 (see e.g., FIG. 11C). Immune cell types associated with survival on day 7 included B-cell subsets (naive, intermediate, memory, and plasmablasts), MAIT-cells, NK-cells, and select T-cell subsets (see e.g., FIG. 11D). These data were leveraged to focus the subsequent analysis on immune cell populations most associated with patient survival.

Cell-Specific Immune Activation Signatures Are Associated With Survival in Critically Ill COVID-19 Patients.

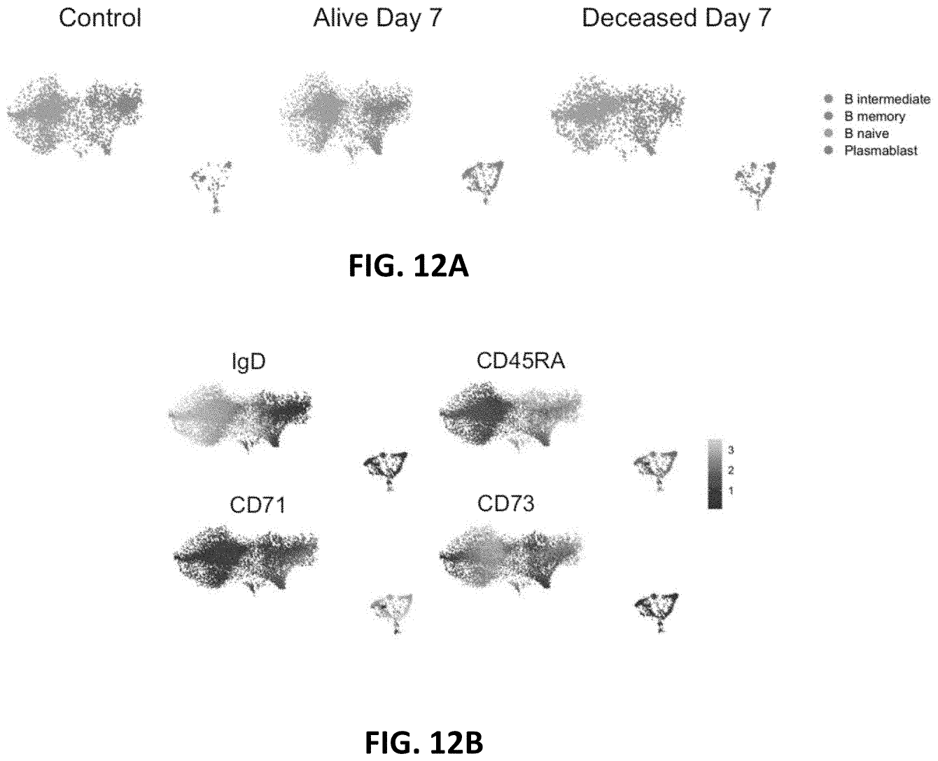

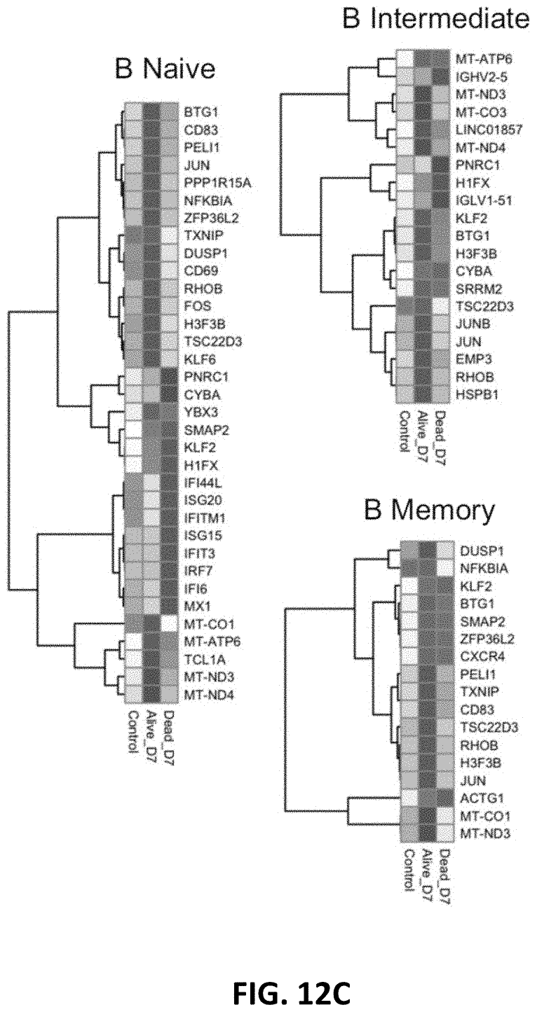

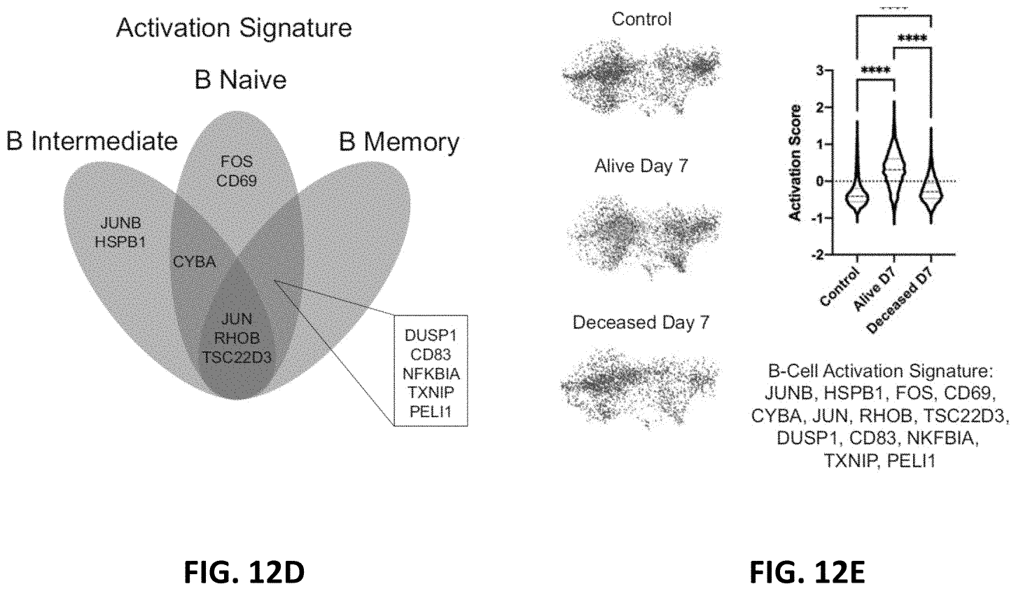

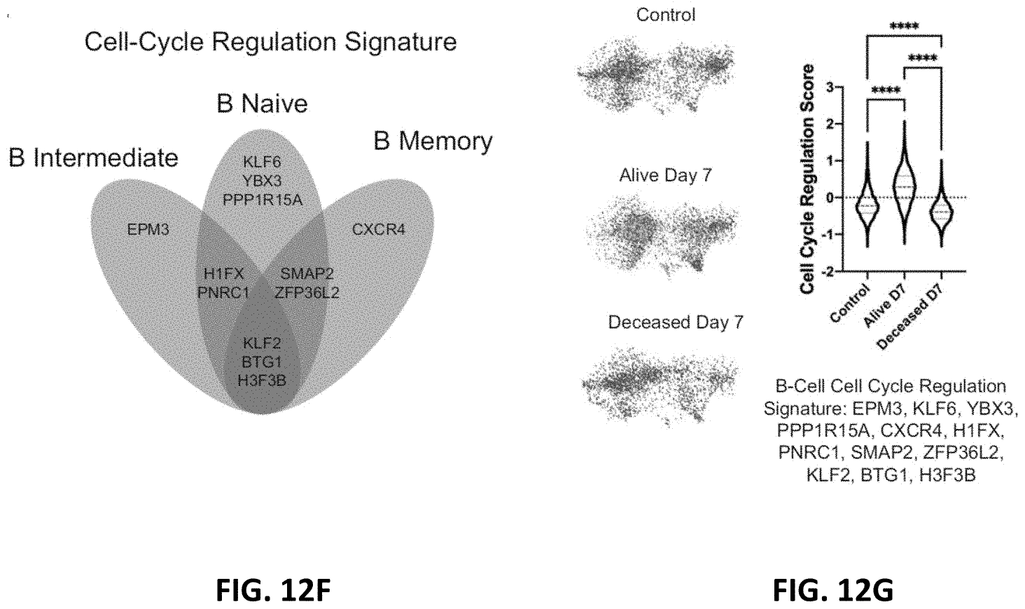

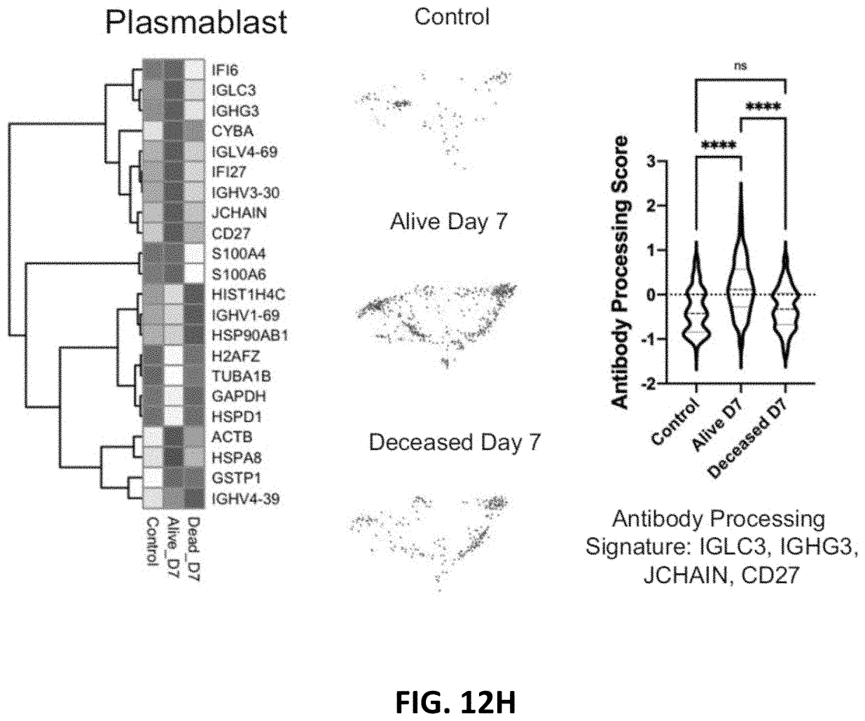

To define the peripheral immune phenotype of patients who survive critical COVID-19, patients who eventually lived or succumbed to infection were compared on day 7. Based on the cell-specific DGE analysis (see e.g., FIG. 11A-FIG. 11D), B-cell subsets were analyzed with higher granularity. UMAP embedding plots of B-naive, B-intermediate, B-memory cells, and plasmablasts revealed evidence of plasmablast expansion in COVID-19 (see e.g., FIG. 12A)—4.0% in control and 12.5% in day 7 COVID-19. Imputed B-cell surface-protein expression from azimuth further validated the cell-specific population definitions (see e.g., FIG. 12B). 35, 20, and 11 differentially expressed genes were identified in naive, intermediate and memory B-cells, respectively, using a log₂ fold-change threshold of 0.58. Genes differentially expressed in patients who survived were indicative of early activation and cell cycle regulation signatures (see e.g., FIG. 12C). Activation gene signatures showed overlap across B-cell subsets with three genes common across all populations (JUN, RHOB, and TSC22D3, see e.g., FIG. 12D). A z-score was computed for the composite signature of all differentially expressed activation response genes and overlaid on the B-cell UMAP embedding, which revealed robust enrichment in B-cells from patients who survived (see e.g., FIG. 12E). Cell cycle regulation genes showed a similar degree of overlap with 3 genes common to each B-cell subset (KLF2, BTG1, and H3F3B, see e.g., FIG. 12F). These cell cycle regulation genes were combined and a z-score composite signature overlaid on the B-cell UMAP embedding demonstrating increased expression of cell cycle regulatory genes in B-cells from patients who survived (see e.g., FIG. 12G). B-cells from patients who eventually succumbed to infection displayed a reduced cell cycle regulatory signature compared to controls (see e.g., FIG. 12G).

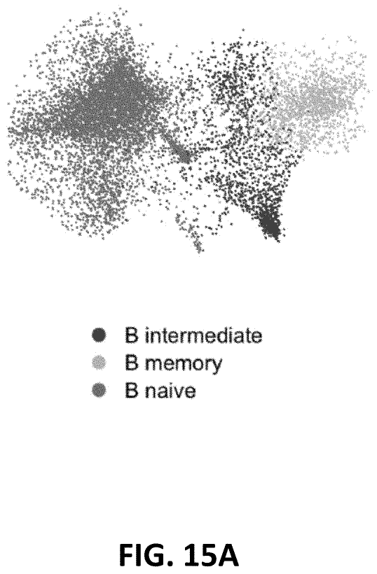

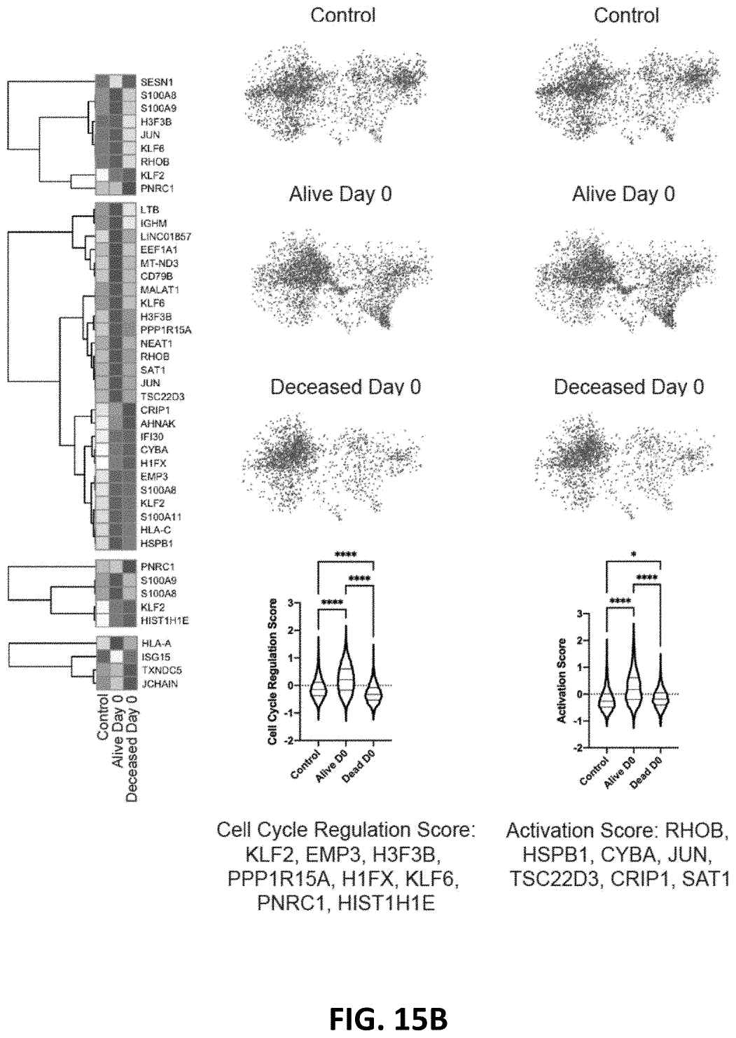

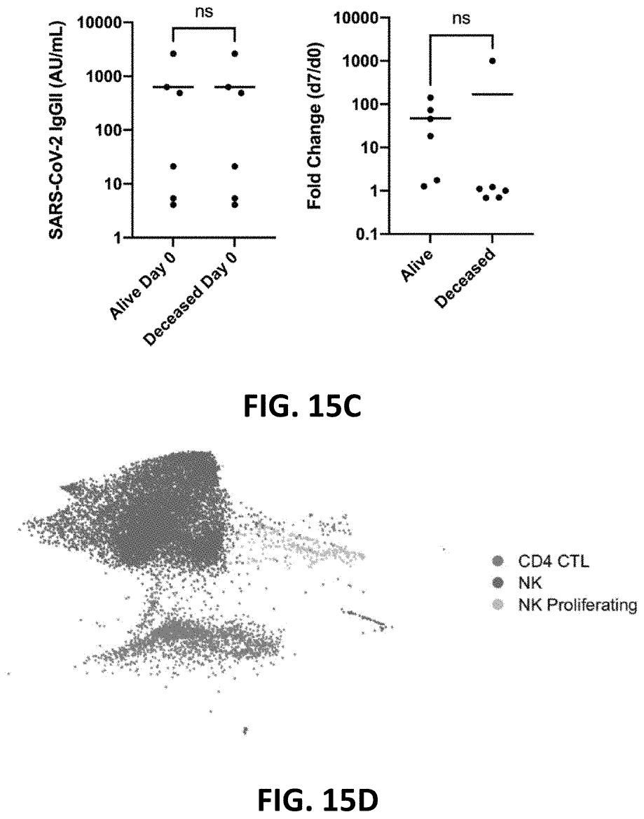

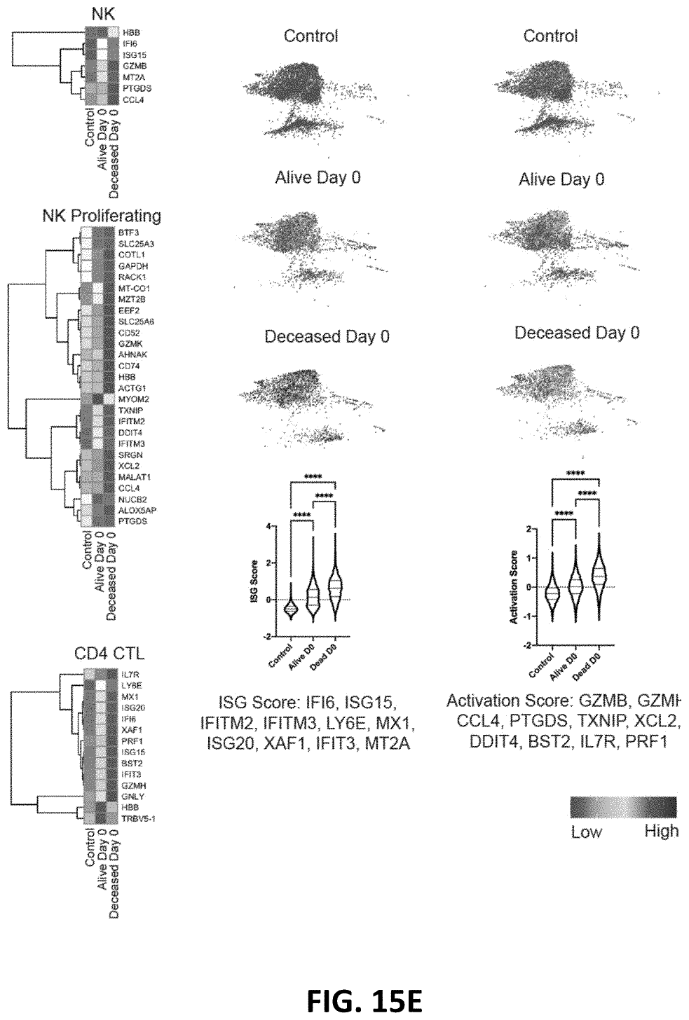

Plasmablast DGE analysis revealed 15 differentially expressed genes between patients who survived versus those who succumbed to COVID-19 (log₂ fold-change>0.58). Differentially expressed genes were enriched in components of antibody processing (IGLC3, IGHG3, JCHAIN, and CD27). This signature was selectively found in patients who survived infection (see e.g., FIG. 12H). SARS-CoV-2 IgG II testing was performed in the COVID-19 cohort at day 0 and 7 and no serology differences were found in the cohorts reinforcing the importance of high-depth transcriptional signature profiling in critical COVID-19 (see e.g., FIG. 15C).

Interferon signaling is increased in COVID-19 patients and thought to contribute to host protection. Whether type I interferon signaling among the B-cells and plasmablasts was associated with survival was explored. Among these populations, naive B-cells and plasmablasts expressed ISGs in COVID-19 patients. Canonical ISGs from the B-naive and plasmablast DGE analyses were combined to create a collective ISG z-score (IFIT3, ISG15, 1FI6, MX1, IFI44L, ISG20, IFITM1, IFI27). Surprisingly, UMAP embedding plots and direct comparison of z-scores showed an increase in ISG expression in B-naive cells from the deceased cohort but an increase in this signature in plasmablasts from patients who survived (see e.g., FIG. 12I). These findings highlight the significance of interferon signaling in distinct cell states and types with respect to survival in critical COVID-19 patients.

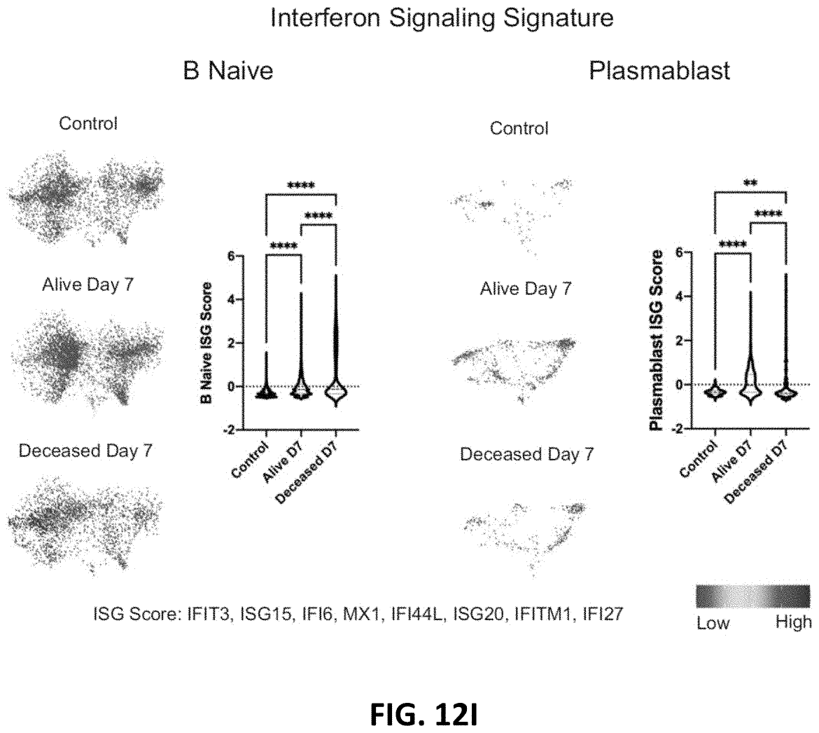

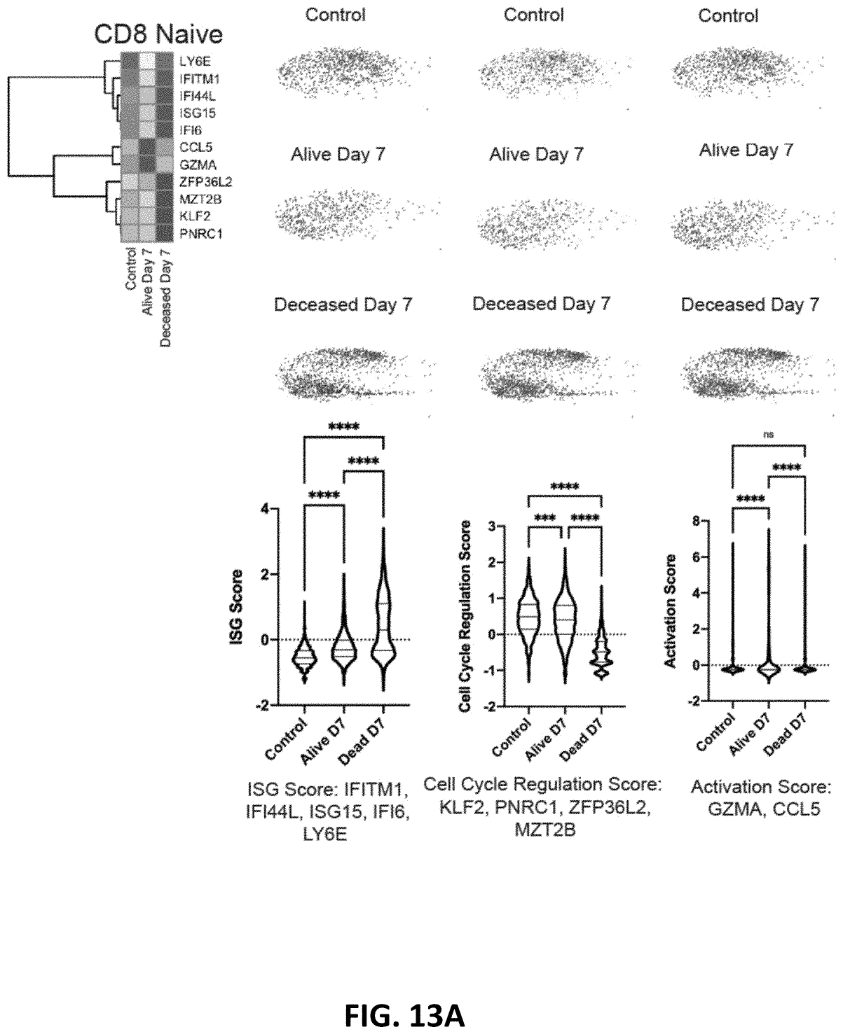

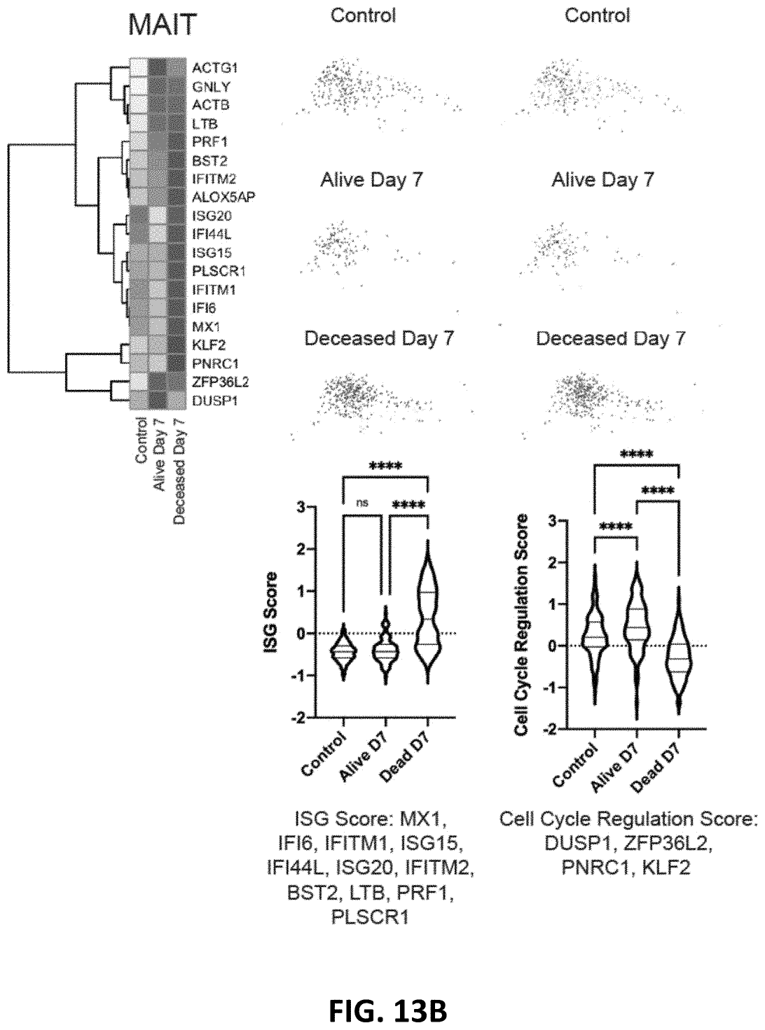

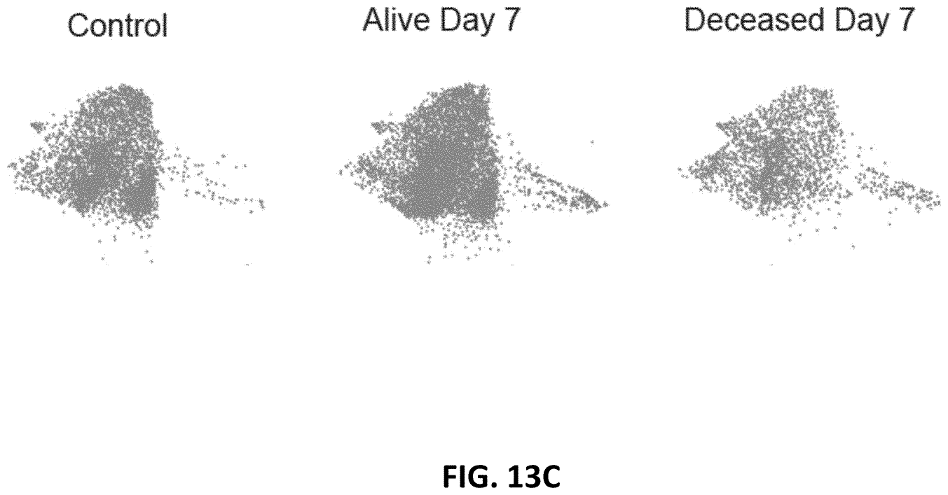

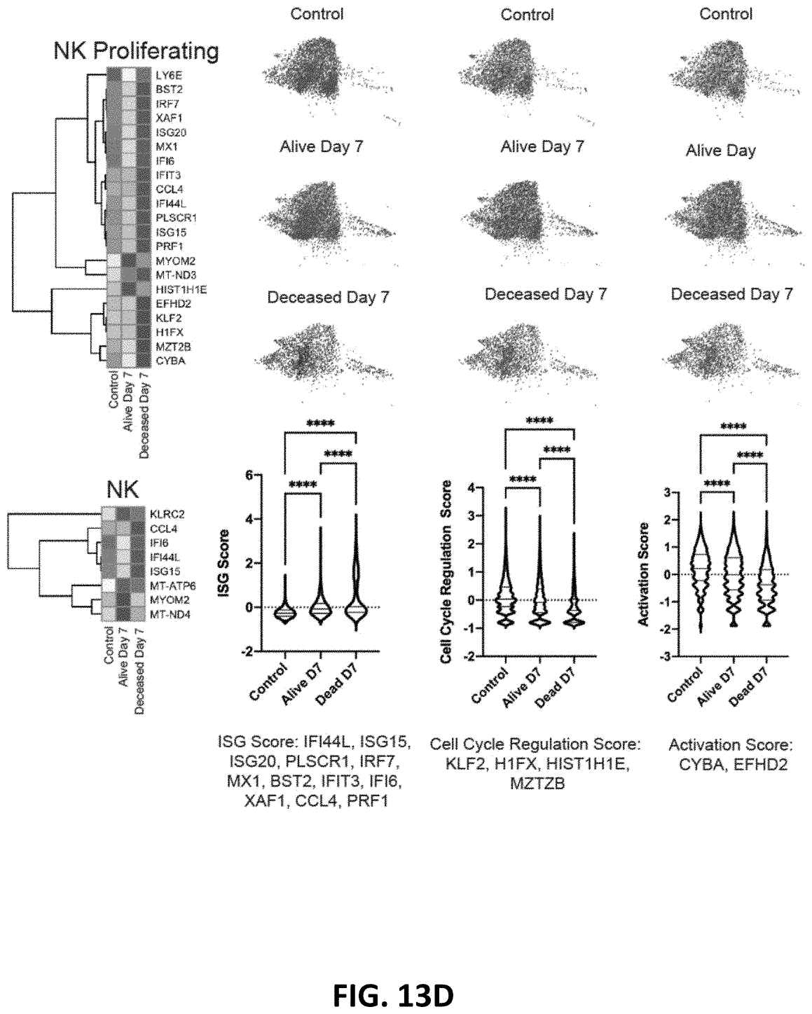

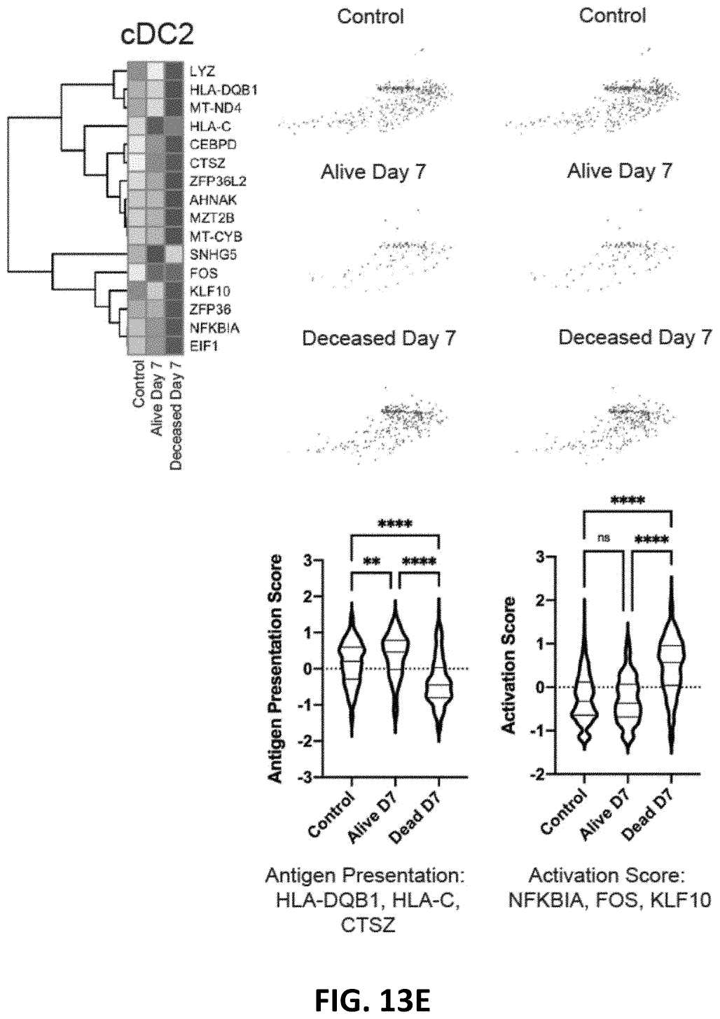

Naive CD8-, NK-, and MAIT-cells showed similar patterns to B-cells in regard to activation and cell cycle regulation gene signatures in alive versus deceased patients with increased z scores in patients who survived (see e.g., FIG. 13A-FIG. 13E). Each of these immune subsets showed increased ISG signatures in the deceased cohort (see e.g., FIG. 13A-FIG. 13E). GZMA and CCL5 expression in naive CD8 T-cells was increased in surviving patients. Analysis of transcriptional signatures associated with survival in cDC2 cells on day 7 revealed several differences. Patients who survived displayed an enrichment for genes associated with antigen-presentation, while patients who died expressed genes associated with cell activation (NFKBIA, FOS, KLF10) (see e.g., FIG. 13A-FIG. 13E). Together these findings identify that cell-specific immune responses including B-cell activation and cell cycle regulation, plasmablast antibody processing, and cDC2 antigen presentation are associated with survival.

Early Cell-Specific Gene Expression Predicts COVID-19 Survival.

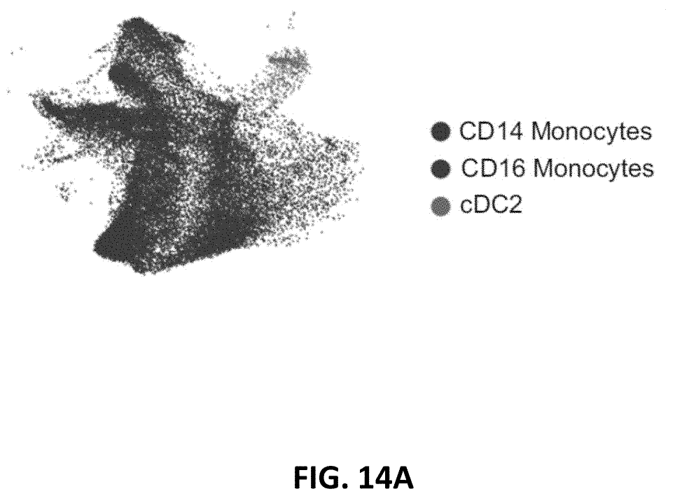

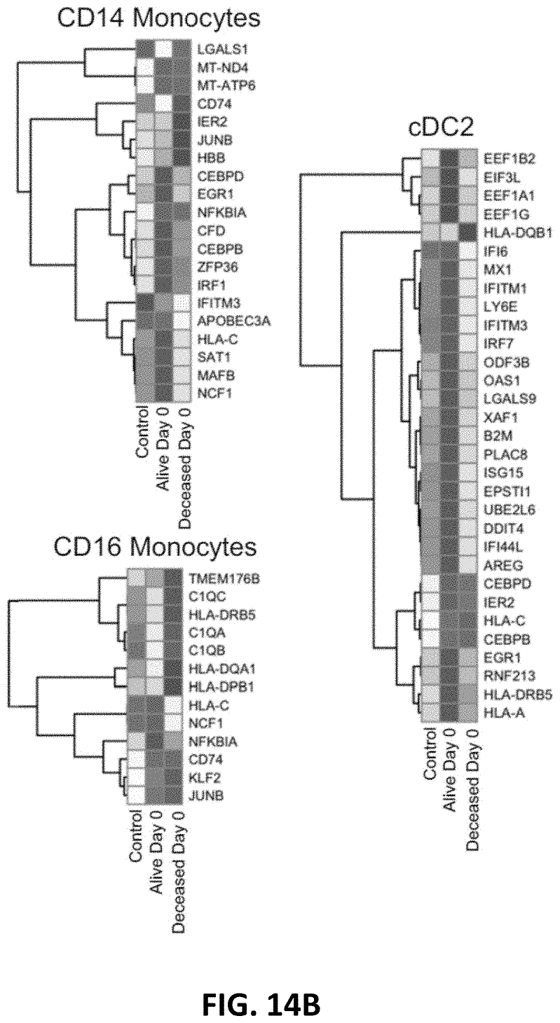

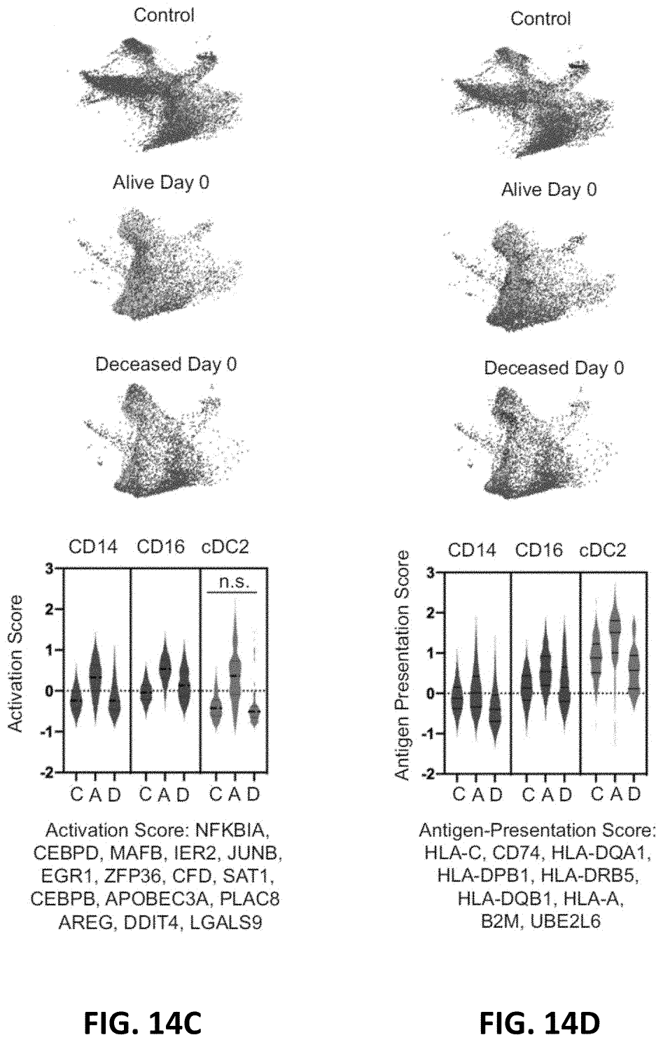

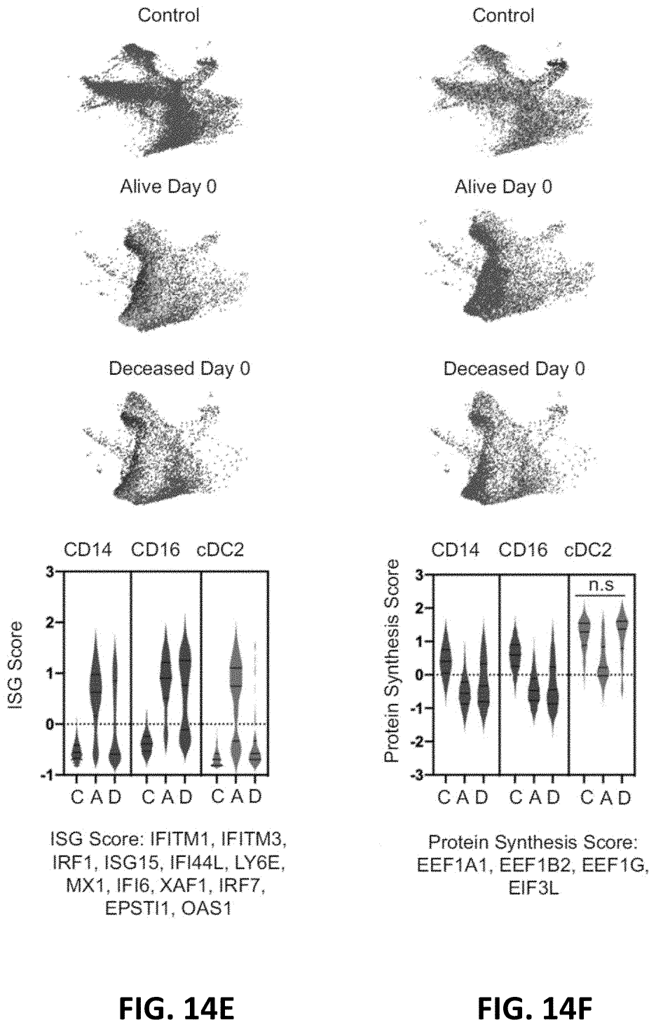

To identify cell-specific transcriptional signatures (and to calculate corresponding gene expression scores) that predict COVID-19 survival, DGE testing and a machine learning model were utilized. Based on the DGE analysis comparing alive and deceased patients on day 0 (see e.g., FIG. 11A-FIG. 11D), CD14 monocytes, CD16 monocytes, and dendritic cells were interrogated with higher granularity (see e.g., FIG. 14A). DGE analysis revealed 11, 9, and 30 genes differentially expressed between patients who survived versus those who succumbed to COVID-19 (log₂ fold-change>0.58) in CD14 monocytes, CD16 monocytes, and cDC2 cells, respectively. CD14 monocytes and cDC2 cells displayed signatures of antigen-presentation and interferon signaling in patients who survived relative to controls and the deceased cohort. CD16 monocytes also displayed signatures of cell activation and antigen-presentation selectively in surviving patients whereas interferon signaling was increased in both patients who survived and died compared to controls (see e.g., FIG. 14B-FIG. 14E). Decreased expression of elongation factor genes (EEF1A1, EEF1B2, EEF1G, EIF3L) was detected in cDC2 cells from patients who survived compared to the other groups. Within CD14 and CD16 monocytes, this elongation factor signature was similarly reduced among surviving and deceased cohorts relative to controls (see e.g., FIG. 14F).

Signatures that predicted survival were also detected in other immune cells. Naive, intermediate, and memory B-cells displayed cell cycle regulation and activation signatures in patients who survived (see e.g., FIG. 15A-FIG. 15B) although SARS-CoV-2 IgGII titers were not different (see e.g., FIG. 15C). CD4 T-cells with cytotoxic activity (CD4 CTLs) and NK-cells displayed enhanced interferon signaling and effector activation markers (GZMB, GZMH, CCL4, XCL2) in patients who succumbed to COVID-19 (see e.g., FIG. 15D-FIG. 15E). These data highlight the early role of adaptive immune cells in the immune response of critically ill COVID-19 patients.

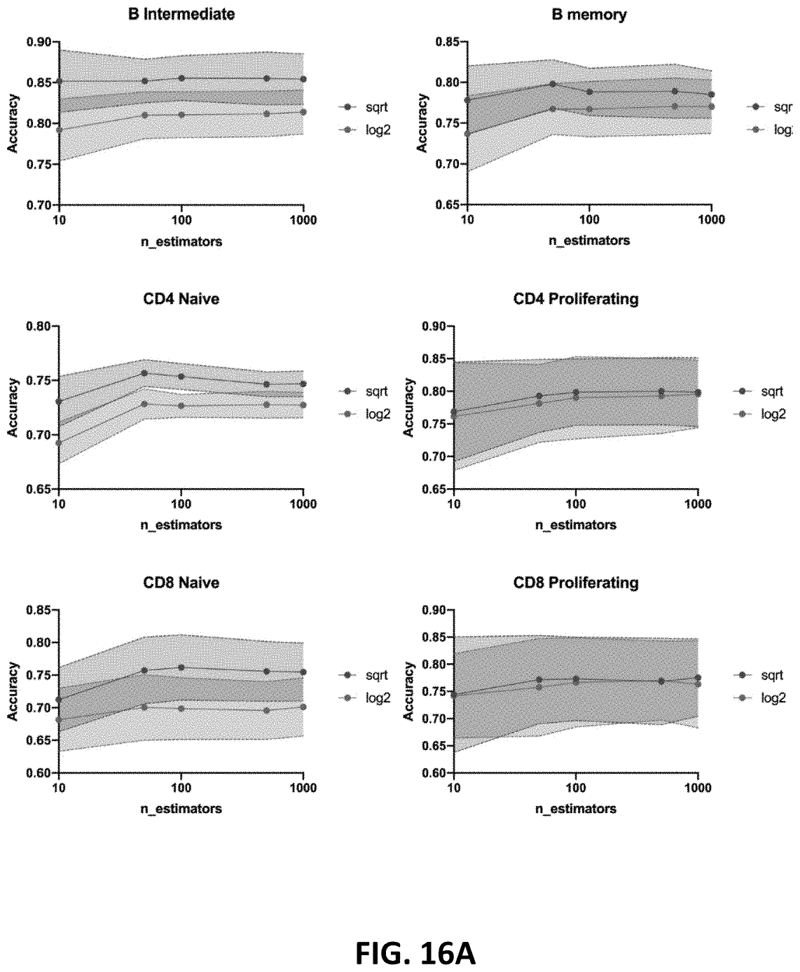

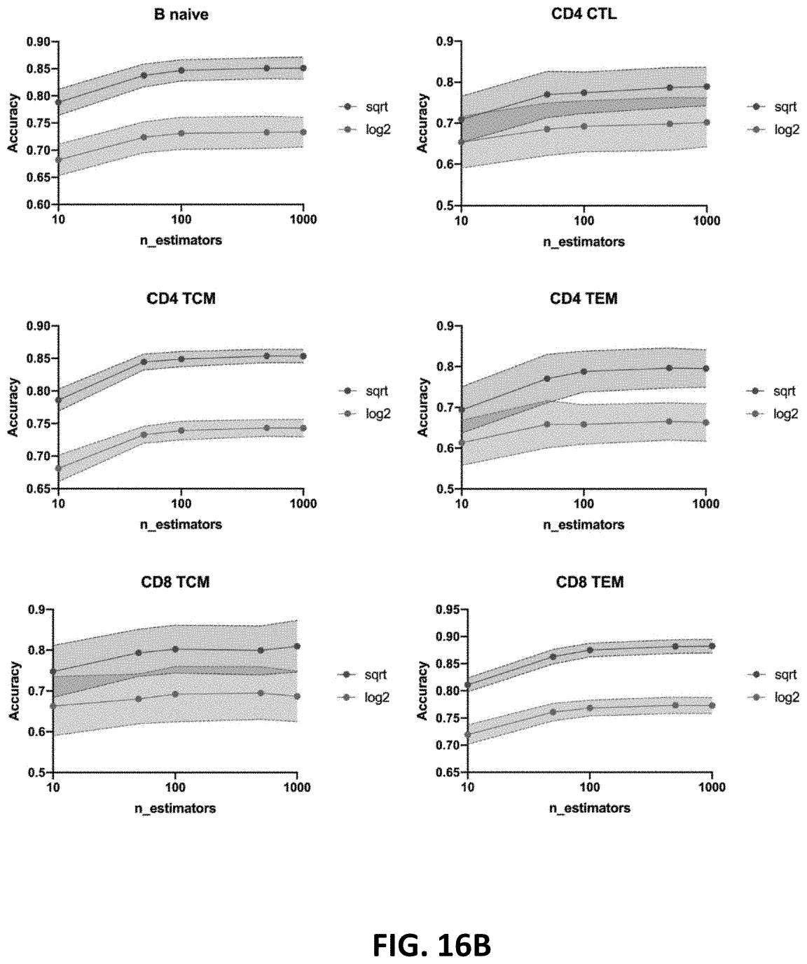

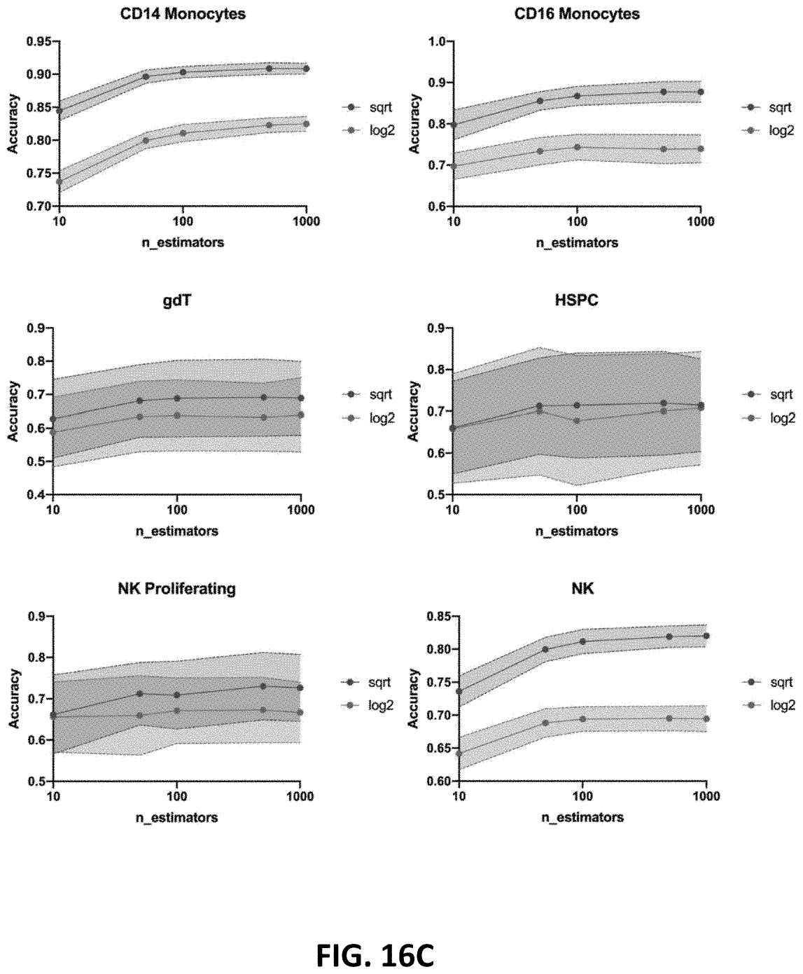

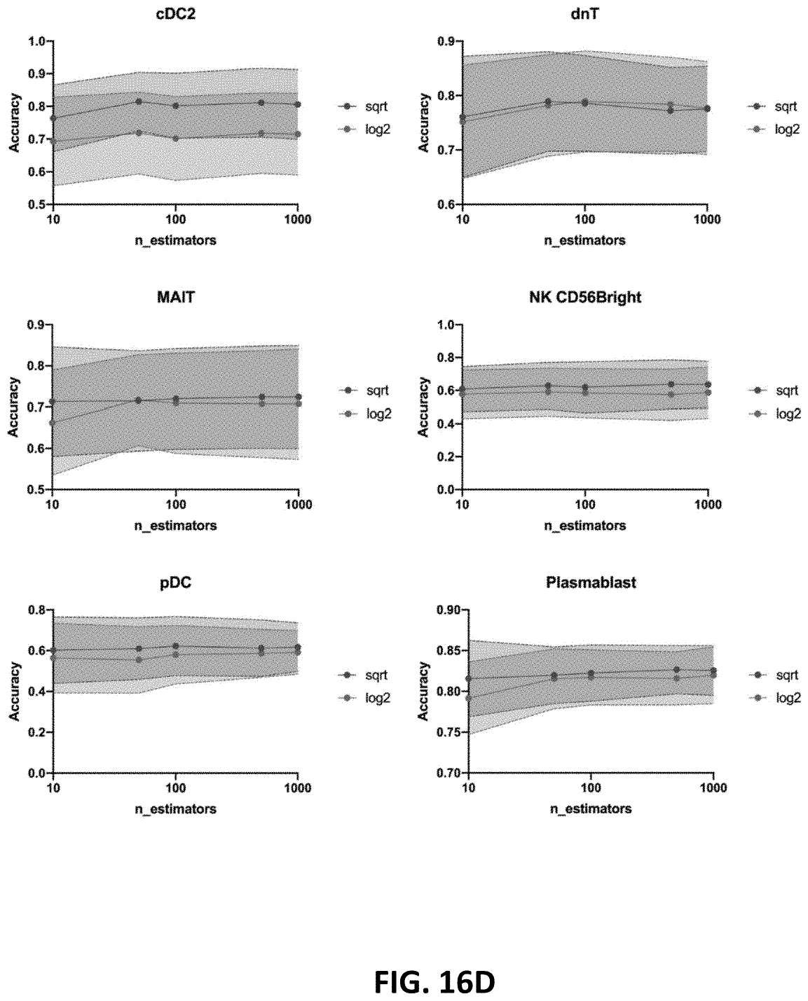

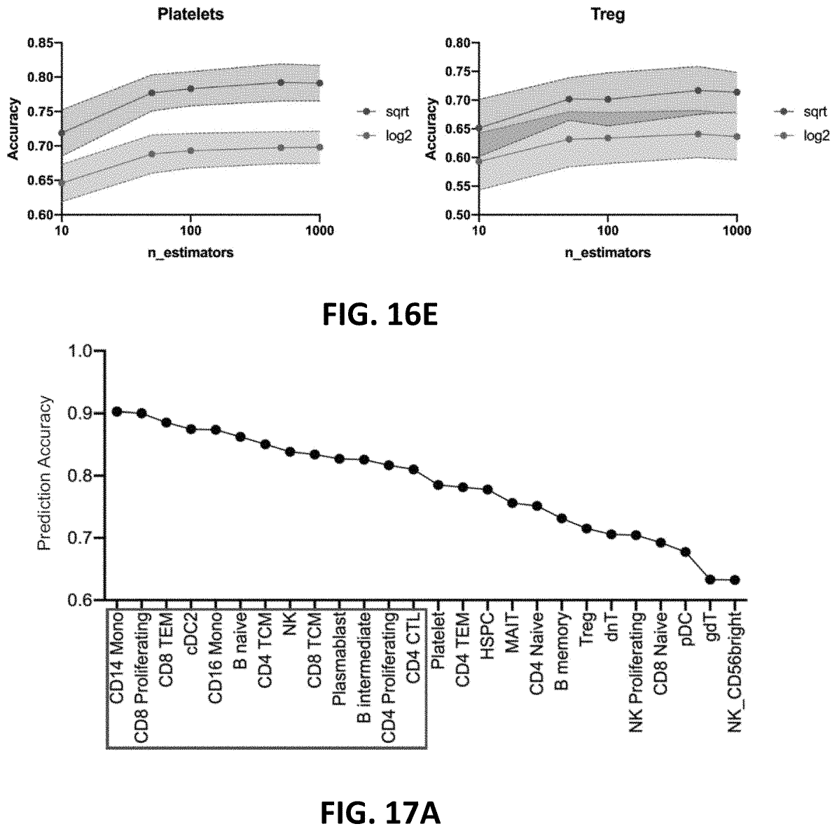

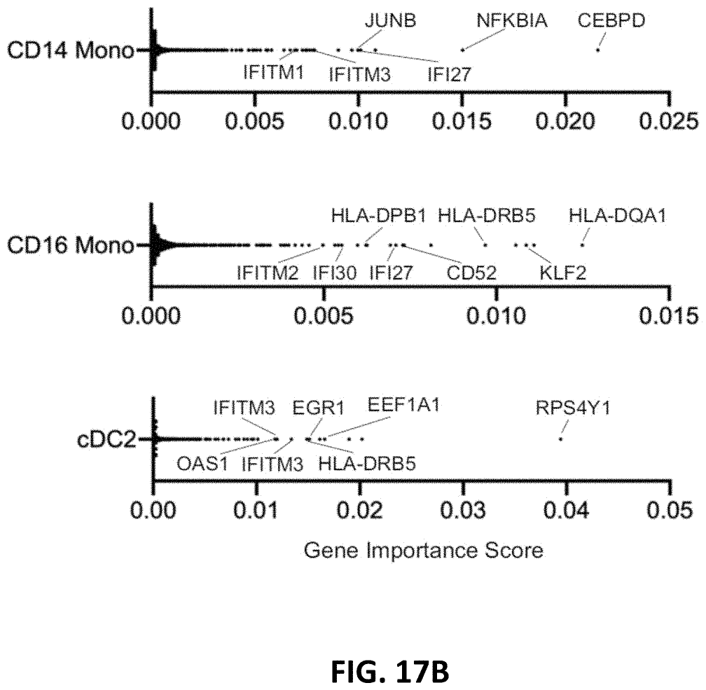

Next, a cell type-specific random forest classifier was used to predict survival based on single-cell transcriptomes. The model was trained on 70% of the cells with 3000 highly variable genes and 10-fold cross-validation was used with 5 trials to determine optimal hyperparameters (see e.g., FIG. 16A-FIG. 16E). These parameters were then used to calculate prediction accuracy on the remaining 30% of cells (see e.g., FIG. 17A). This random forest classifier prediction showed that CD14 monocyte transcriptomes exhibit the strongest prediction of mortality with a 90% accuracy and 0.97 ROC. cDC2, CD8 T-cells, and CD16 monocytes also show high predictive power (>85%). To delineate which genes contribute most to survival prediction accuracy, ranked feature importance scores were examined for these monocytes and dendritic cells (see e.g., FIG. 17B). ISGs such as IFI27, IFITM1, IFITM3, IFITM2, IFI30, and OAS1 were identified as key in predicting survival in CD14 monocytes, CD16 monocytes, and cDC2 cells.

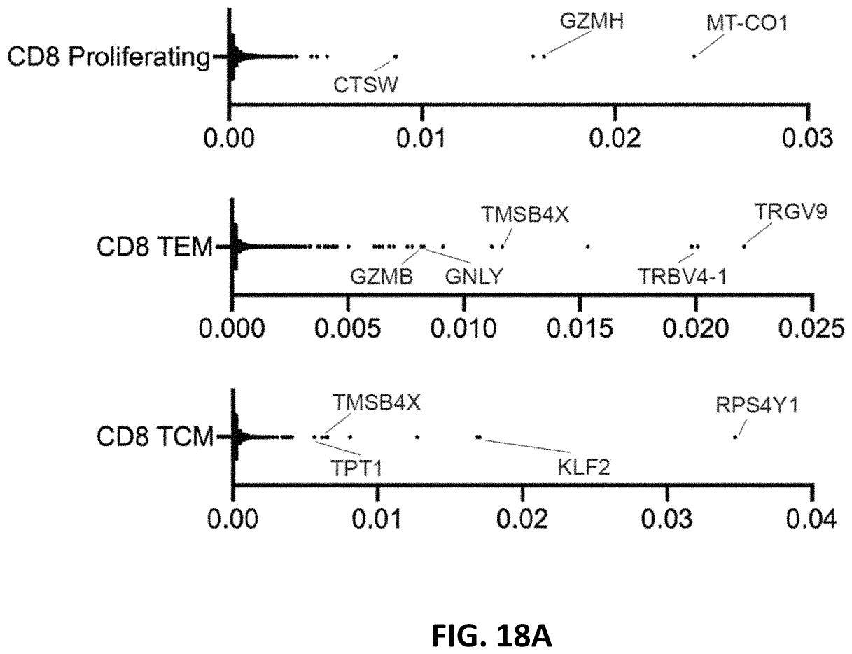

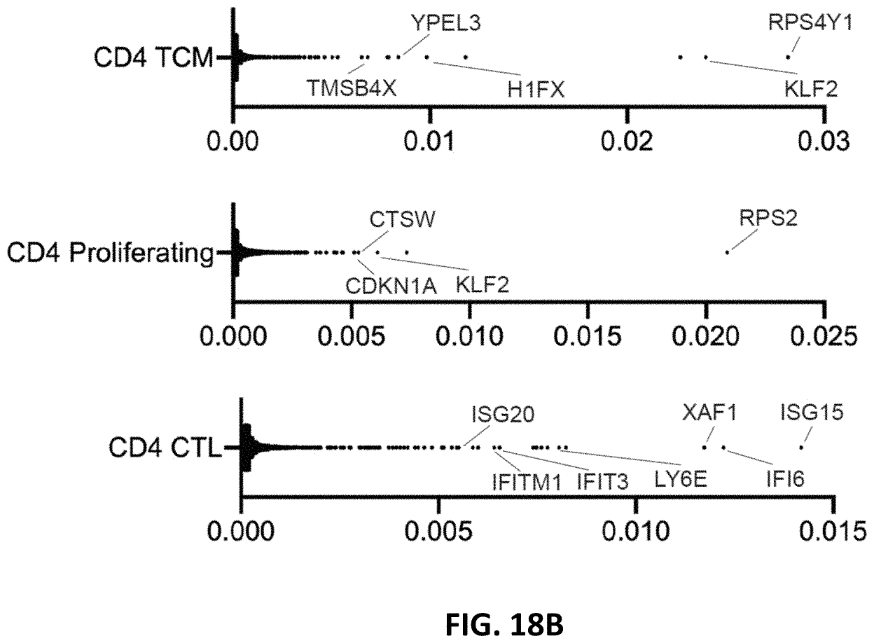

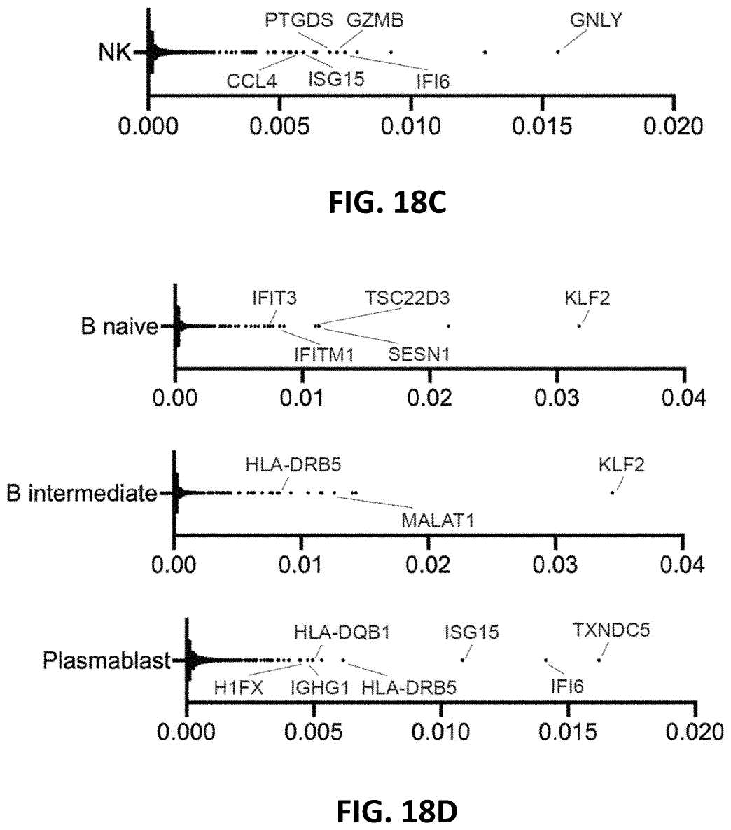

In CD14 monocytes, early-response genes such as NKFBIA, JUNB, and CEBPD were among the highest ranked features (see e.g., FIG. 17B), while antigen-presenting genes (HLA-DQA1, HLA-DRB5, HLA-DPB1) were the most predictive in CD16 monocytes (see e.g., FIG. 17B). In cDC2 cells protein synthesis genes such as elongation factors (EEF1A1, EEF1B2) and ribosomal genes (RPS4Y1) were also highly ranked (see e.g., FIG. 17B). Adaptive immune cell populations also showed strong predictive power. Early response and cell cycle regulation genes were most predictive among CD8 and CD4 T-cell subsets (see e.g., FIG. 18A-FIG. 18B). Consistent with the differential gene expression findings, CD4 CTL and NK cells show ISGs as strongly predictive of survival (see e.g., FIG. 18C-FIG. 18D), and antigen-presentation and ISGs were among the key predictive features within the B-cell subsets and plasmablasts (see e.g., FIG. 18D).

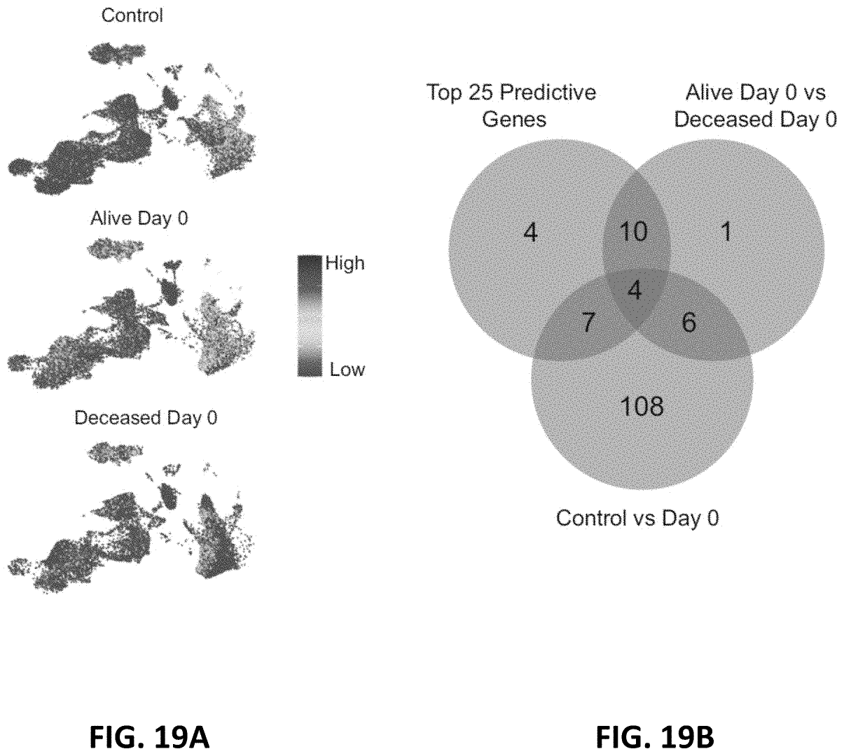

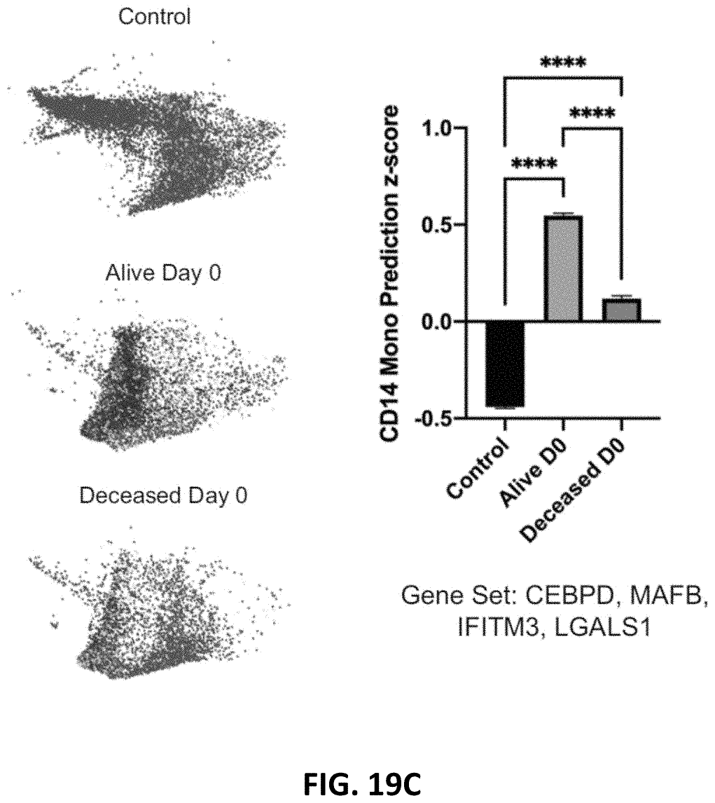

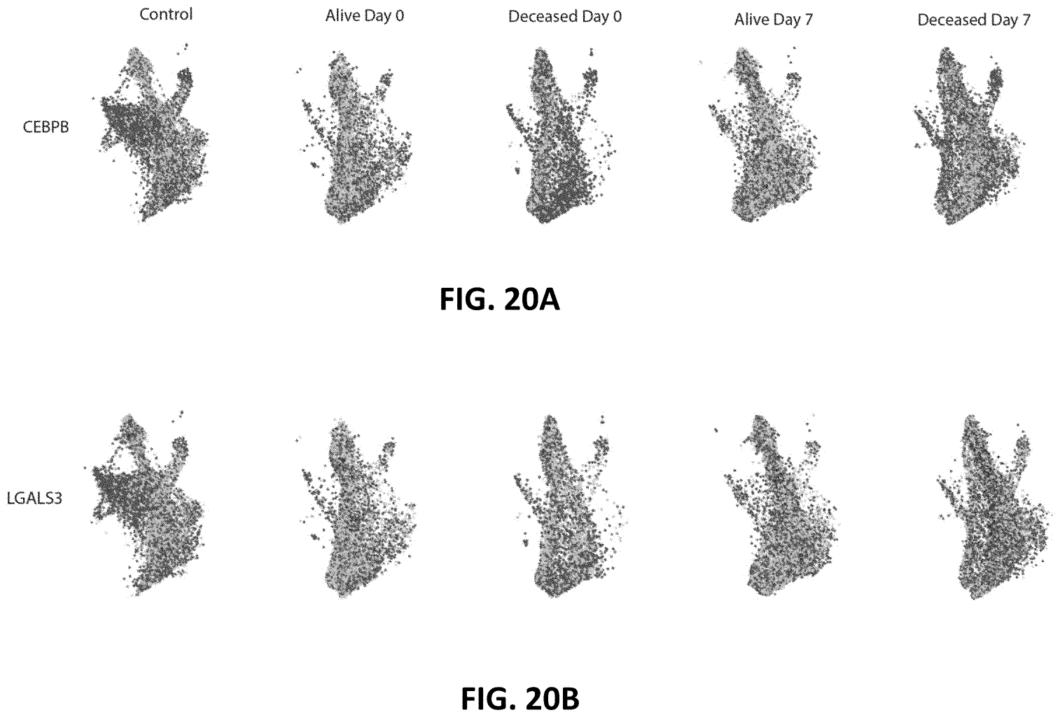

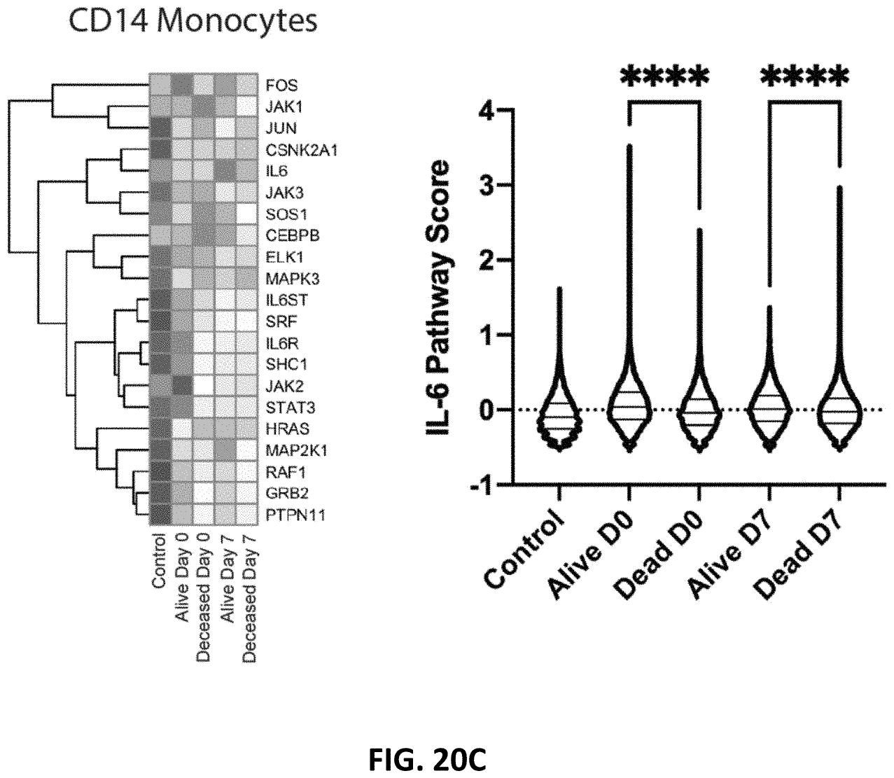

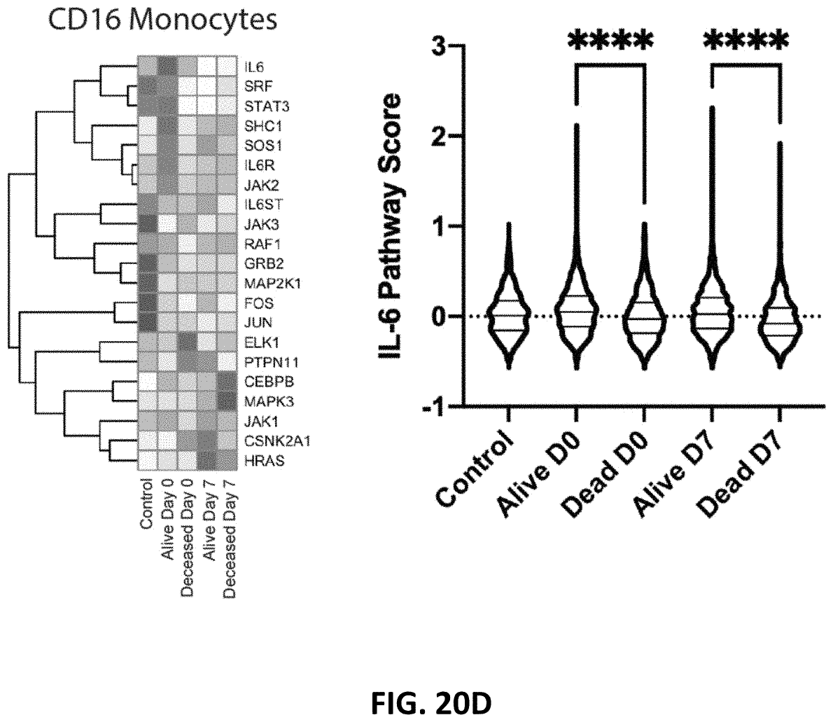

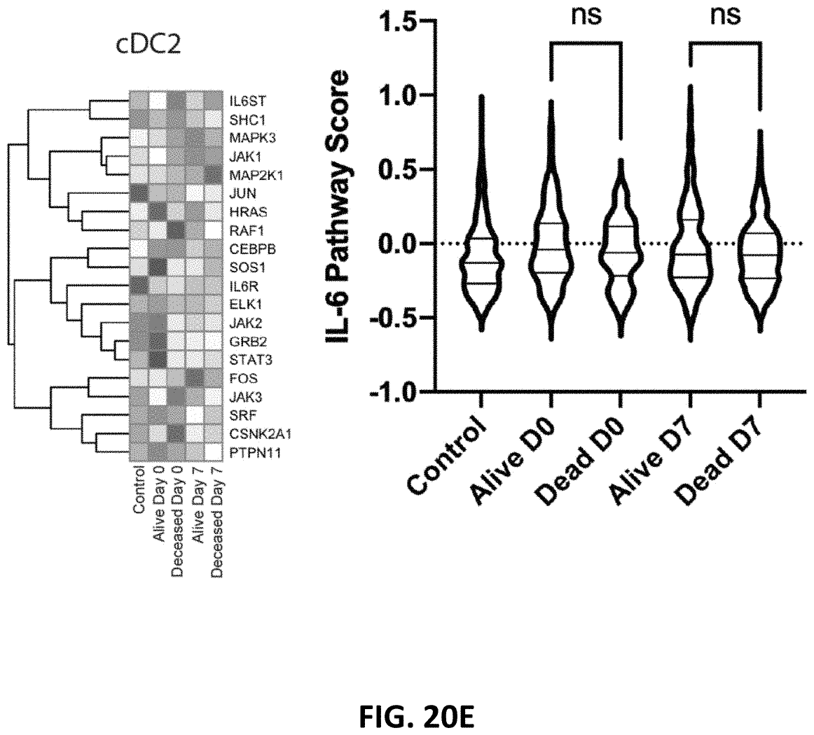

To verify the ranked features manifest transcriptional differences, the intersecting genes from the top 100 feature importance genes for CD14 monocytes, CD16 monocytes, and cDC2 cells were taken and a combined z-score was plotted on a UMAP embedding for the entire dataset (see e.g., FIG. 19A). This visualization showed that the combined gene signature is enriched in the alive cohort and localized to the monocytes and dendritic cells (see e.g., FIG. 19A). This validation bolsters the cell type-specific nature of the signatures discovered in the model. Finally, to build a refined gene signature of most significance, the intersecting genes for CD14 monocytes using the top 25 predictive features, alive versus deceased cohort DGE at day 0, and control versus all patients DGE at day 0 were taken. This integrated approach yielded a list of 4 intersecting genes: CEBPD, MAFB, LGALS1, and IFITM3 (see e.g., FIG. 19B). UMAP embedding analysis showed that the composite z-score for these four genes demonstrates a strong enrichment among patients who survived COVID-19 (see e.g., FIG. 19C). As CEBPB and LGALS3 are known regulators of IL-6 signaling, IL-6 signaling genes were further interrogated in the dataset. CEBPB was strongly enriched in the alive cohort (see e.g., FIG. 20A) at day 0 and 7 while LGALS3 was enriched in the alive cohort at day 0 and the deceased cohort at day 7 (see e.g., FIG. 20B). A combined IL-6 pathway score was calculated, which was elevated in the alive cohort in CD14 monocytes and 16 monocytes at both day 0 and day 7 (see e.g., FIG. 20C-FIG. 20D) but not cDC2 cells (see e.g., FIG. 20E), suggesting that IL-6 signaling may have a protective role in the most critically ill patients.

Cross-Validation of Random Forest Classifier Framework.

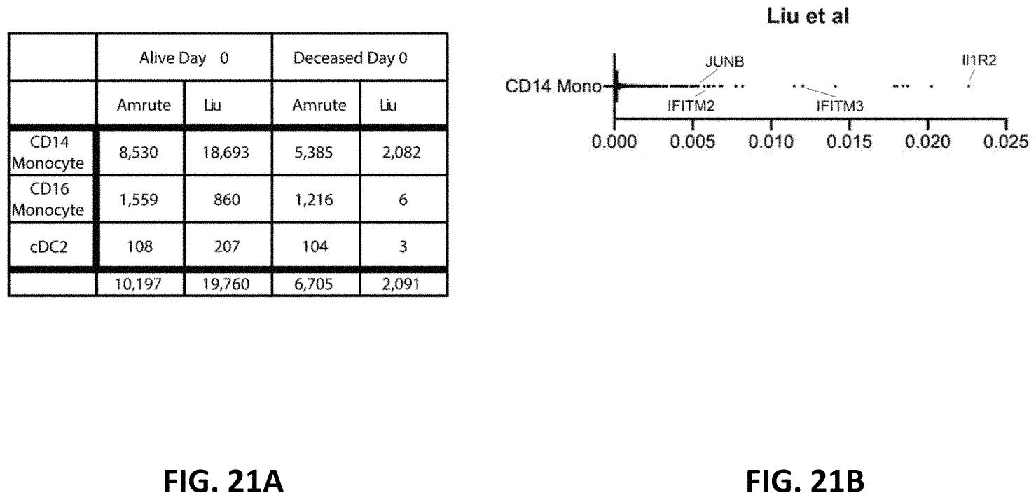

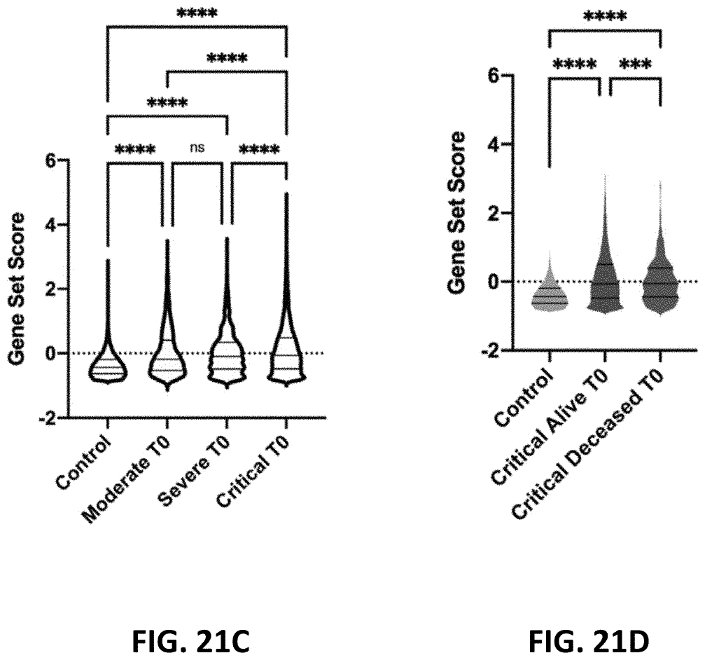

To robustly validate the transcriptional signature found via random forest classification and differential gene analysis, a previously published CITE-seq dataset was leveraged. This dataset incorporated 18,693 CD14 monocytes from 21 patients who lived and 2082 from 4 patients who died (see e.g., FIG. 21A). To assess the applicability of the random forest framework, the algorithm was trained and tested on the CD14 monocytes from this critically ill cohort at timepoint 0. The algorithm predicted mortality in this dataset with 94% accuracy and identified several genes in common with the dataset—specifically, IFITM3 and JUNB were some of the key features associated with patient survival outcome (see e.g., FIG. 21B). As this dataset subclassified severity of illness, a z-score was calculated for the intersecting gene list (CEBPD, MAFB, LGALS1, and IFITM3) and gene set expression enrichment was found in critically ill patients compared to controls and lesser severity illness patients; however, there was no difference between moderate and severe patients at time 0 (T0, see e.g., FIG. 21C). Finally, this z-score was calculated in their healthy controls, surviving critically ill and deceased patients. This signature was enriched among critically ill patients who survived infection relative those who died and controls (see e.g., FIG. 21D). These findings validate the computational framework and serve as an independent benchmark for the significance of the refined gene list in predicting outcomes among critically ill COVID-19 patients.

Discussion

In this study, scRNA-seq and CITE-seq mapping of PBMCs were used to dissect longitudinal transcriptional differences associated with survival in critical COVID-19 patients. Prior multiomic studies have profiled large cohorts and delineated key immunological findings in COVID-19; however, to date there does not appear to be a robust dataset, which identifies transcriptional signatures (e.g., gene expression scores computed from expression levels of at least two genes) associated with survival in critically ill patients, as described herein.

Broadly cell cycle regulation, cell-specific activation markers, and antibody processing genes within B-, T-, and NK-cell subsets were found to be preferentially increased in patients who survived infection. Common early-response cell activation markers included JUN and RHOB. Patients who survived displayed expression of ISGs in plasmablasts. Similar to prior studies, plasmablast expansion was found in all critical COVID-19 patients relative to controls. Later (day 7) signatures associated with mortality were also identified. cDC2 cells from patients who ultimately died showed an increase in inflammatory activation markers (NFKBIA, FOS, KLF10). Naive B-, naive CD8 T-, NK-, and MAIT-cells displayed a robust interferon signature in these patients.

To elucidate signatures that predicted survival early in critical COVID-19, multiple approaches were used herein. First, DGE analyses were used to isolate transcriptional differences between specific cell types in the alive and deceased cohort at day 0. Monocyte subsets, cDC2 cells, and B-cell subsets were markedly changed in genes associated with activation, antigen-presentation, and interferon responses in patients who survived infection. In contrast, CD4 CTL T-cells and NK-cell subsets displayed increased expression of effector activation markers (GZMB, GZMH, CCL4, XCL2) and ISGs in patients who succumbed to COVID-19. These findings bolster monocyte interferon response findings from previous studies. Similarly, prior studies have also shown heightened cytotoxic T-cell activation signatures in COVID-19. Second, a random forest classifier model was trained within each cell type to predict survival using its cell-specific transcriptome. CD14 monocytes, CD16 monocytes, and cDC2 cells were among the cell types with the strongest predictive power; CD14 monocytes had 90% accuracy. Finally, a refined gene signature identified from both DGE testing and random forest classification in CD14 monocytes was constructed. This analysis identified a signature for 4 genes, CEBPD (−0.93 log₂ FC), MAFB (0.83 log₂ FC), IFITM3 (0.55 log₂ FC), and LGALS1 (−0.53 log₂ FC), that was markedly enriched in CD14 monocytes in patients who ultimately survived infection.

The gene list was validated in an independent dataset from Liu et al. within the critical cohort at day 0 and enrichment of CEBPD, MAFB, LGALS1, and IFITM3 was found among those who survived. Furthermore, the CD14 monocytes in Liu et al. critically ill cohort at day 0 were trained and tested, and consistent genes associated with mortality were found. The predictive genes CEBPD and LGALS1 regulate IL-6 signaling, and this pathway has been targeted clinically by monoclonal antibody administration with varying success. Further interrogation of the IL-6 signaling pathway showed that IL-6 signaling is enriched in monocytes in patients who survive infection, particularly at early time points.

This finding highlights a potentially protective role of IL-6 signaling in critically ill patients and demonstrates the importance of further investigation into IL-6 signaling targeting therapies. There are multiple future directions for this study. First, future studies may explore gender, age, race, and comorbidities. Second, the analysis herein is focused on transcriptomic data, but there may be added benefit of utilizing multiomic mapping in multiple tissue contexts. Future studies may be used to further validate the findings herein, understand their generalizability to patients with differing disease severity, and gauge the added importance of demographic variables, epigenomic, and proteomic predictors of survival.

Herein is provided a longitudinal transcriptomic reference among critically ill COVID-19 patients and insight into the cell-specific immunological mechanisms associated with survival among critically ill COVID-19 patients using peripheral blood mononuclear cells transcriptomics and random forest classification. Early key molecular cell type-specific signatures that predict mortality were delineated, which may allow early risk stratification and provide insights into immune mechanisms most critical for survival in the sickest patient population.

Methods

Subject Selection Criteria and Specimen Collection.

This study complied with all relevant ethical regulations and utilized samples obtained from the Washington University School of Medicine's IRB approved WU350 study, a COVID-19 biorepository, under which patient consent was provided. Patient samples were selected based on severity of illness as defined by admission to the intensive care unit. Those selected had availability of PBMC samples at both day 0 and day 7 of enrollment and were demographic matched into eventual surviving and deceased cohorts. Control PBMCs were obtained from Washington University's Alzheimer's Disease Research Center specimen collection from age-matched healthy people without dementia.

PBMC Isolation and Single-Cell RNA Sequencing.

Cryopreserved PBMCs were thawed and washed with HBSS with 2 mM EDTA and 0.04% LPS-free BSA twice. Cell viability was assessed by trypan blue staining and samples with >80% viability were submitted to the McDonnell Genome Institute at Washington University in St. Louis. cDNA was prepared after the GEM generation and barcoding, followed by the GEM-RT reaction and bead cleanup steps. Purified cDNA was amplified for 10-14 cycles before being cleaned up using SPRI select beads. Samples were then run on a Bioanalyzer to determine the cDNA concentration. GEX libraries were prepared as recommended by the 10× Genomics Chromium Single Cell V(D)J Reagent Kits (v1 Chemistry) user guide with appropriate modifications to the PCR cycles based on the calculated cDNA concentration. For sample preparation on the 10× Genomics platform, the Chromium Single Cell 5′ Library and Gel Bead Kit (PN-1000006), Chromium Single Cell A Chip Kit (PN-1000152) and Chromium Dual Index Kit TT Set A (PN-1000215) were used. The concentration of each library was accurately determined through qPCR utilizing the KAPA library Quantification Kit according to the manufacturer's protocol (KAPA Biosystems/Roche) to produce cluster counts appropriate for the Illumina NovaSeq6000 instrument. Normalized libraries were sequenced on a NovaSeq6000 S4 Flow Cell using the XP workflow and a 151×10×10×151 sequencing recipe according to manufacturer protocol. A median sequencing depth of 50,000 reads/cell was targeted for each Gene Expression Library.

ScRNA-Seq Analysis Pipeline.

The sequenced fastq files were aligned to a human reference genome (GRCh38) using the CellRanger Software (v4.0, 10× Genomics) to generate feature-barcoded count matrices. Subsequent analysis was performed using the R Seurat v4.0.0 package. The following quality control steps were performed to filter the count matrices: 1. genes expressed in <3 cells and cells expressing fewer than 200 genes were removed; 2. Cells expressing >5000 genes were discarded as these could be potential multiplet events where more than a single cell was encapsulated within the same barcoded GEM; 3. Cells with >10% mitochondrial content were filtered out as these were deemed to be of low-quality. Normalization and variance-stabilization of raw counts was performed using SCTransform to find 3000 variably expressed genes and percentage mitochondrial reads were regressed out. The normalized R object was used for subsequent azimuth mapping and differential expression testing.

Mapping scRNA-seq data to a CITE-seq reference using azimuth. The normalized scRNA-seq PBCM dataset (query) was mapped onto a CITE-seq reference of 162,000 PBMCs measured with 228 antibody derived tags. First, anchors were found between the reference and the query using the FindTransferAnchors function with a precomputed supervised principal component analysis transformation and 50 dimensions. Next, each cell in the query was annotated using reference-defined cell states and surface-protein expression was imputed from the reference using the Map-Query function. Finally, a query dataset was computed out onto the reference precomputed UMAP embedding. Accurate cell type annotation was verified using azimuth computed cell state prediction scores and expression of canonical marker genes within each cell state. To further confirm cellular identity, the FindAllMarkers function was used with default parameters and a Wilcoxon rank-sum test to generate a differential expression gene list for all annotated clusters. The reference and query datasets were merged and a new UMAP embedding de novo was recomputed to delineate new cell types in the query not included in the reference. Despite filtering out cells expressing >5000 genes, azimuth detected several doublets, which were removed. Cells annotated as erythrocytes were also removed from the parent R object. For all subsequent analysis, the recomputed UMAP embedding was used for visualization.

Differential expression testing. The normalized and annotated Seurat object was split into each cell type and the FindAllMarkers function was used with default parameters and a Wilcoxon rank-sum test to find differentially expressed genes between the following conditions: Control vs critical COVID-19 Day 0, Control vs critical COVID-19 Day 7, critical COVID-19 Day 0 vs critical COVID-19 Day 7, and Alive Day 0 vs Deceased Day 0 critical COVID-19. Genes with an adjusted p-value <0.05 and log₂ FC>0.50 were deemed significant. Cell states with the most statistically significant different genes were further interrogated. Heatmaps of statistically significant differentially expressed genes (adjusted p-value<0.05 and log₂ FC>0.50) were generated using bulk RNA expression of normalized counts with the AverageExpression( ) function in R for each condition. Gene set module z scores were calculated by grouping statistically significant differentially expressed genes into biologically relevant sets. SARS-CoV-2 antibody testing. Serological testing was performed using the AdviseDx SARS-CoV-2 IgG II assay on an Architect (Abbott Laboratories, #H18575R01) according to the manufacturer's instructions. This assay utilizes a two-step chemiluminescent microparticle that detects IgG antibodies to the RBD domain of the viral Spike protein semi-quantitatively. A result ≥50 AU/mL is considered positive.

Random forest classification. To predict survival in critical COVID-19 from early transcriptional data a random forest classifier was trained using the scikit-learn package in Python v3. The parent R object was subsetted to get the Alive and Deceased Day 0 cells and cell clusters with fewer than 100 cells (ASDC, ILC, cDC1) were discarded from subsequent analysis. Normalized SCTransform RNA counts for the 3000 most highly variable genes were used as features and “Alive” or “Deceased” was used as the label. A random forest classifier model was trained for each cell cluster and a prediction accuracy was calculated in a test dataset to assess importance of each cell type in predicting survival in the context of critical COVID-19. The dataset was split into a train and test set (70% train and 30% test) and the training data was used to optimize the hyperparameters for the Random Forest Classifier. Hyperparameter optimization was performed on the number of estimators (10, 50, 100, 500, 1000) and max features (log and sqrt) through a grid search with 10-fold cross-validation and 5 repeats (50 trials per iteration). Using the optimal hyperparameters for each cell type a random forest classifier was trained and then tested to calculate prediction labels (“Alive” or ‘Dead”) in the test dataset. The sklearn package was used to build a confusion matrix and the prediction accuracy was calculated to compare “cell importance”. For each cell type a list of features was generated and ranked by the feature importance score in the random classifier model.

What is claimed is:

1. A method of determining a risk of mortality from critical COVID-19 for a subject in need thereof, the method comprising:
obtaining a biological sample from the subject;
measuring an expression level of at least two genes selected from:
APOBEC3A, AREG, B2M, BST2, CCL4, CD52, CD74, CDKN1A, CEBPB, CEBPD, CFD, CRIP1, CTSW, CYBA, DDIT4, DDIT4, EEF1A1, EEF1B2, EEF1G, EGR1, EIF3L, EMP3, EPSTI1, GNLY, GZMB, GZMH, GZMI, H1FX, H3F3B, HIST1H1E, HLA-A, HLA-C, HLA-DPB1, HLA-DQA1, HLA-DQB1, HLA-DRB5, HSPB1, IER2, IFI27, IFI44L, IFI6, IFIT3, IFITM1, IFITM2, IFITM3, IGHG1, IL7R, IRF1, IRF7, ISG1, ISG15, ISG20, JUN, JUNB, KLF2, KLF6, LGALS1, LGALS9, LY6E, MAFB, MALAT1, MT2A, MT-CO1, MX1, NFKBIA, OAS1, PLAC3, PNRC1, PPP1R15A, PRF1, PTGDS, RHOB, RPS2, RPS4Y1, SAT1, SESN1, TMSB4X, TPT1, TRBV4-1, TRGV9, TSC22D3, TXNDC5, TXNIP, UBE2L6, XAF1, XCL2, YPEL3, or ZFP36;
computing a gene expression score from the expression level of the at least two genes;
comparing the gene expression score to a reference score computed from a reference sample; and
determining the subject has a high risk of mortality from critical COVID-19 when the gene expression score is significantly different from the reference score; or
determining the subject has a low risk of mortality from critical COVID-19 when the gene expression score is not significantly different from the reference score.

2. The method of claim 1, wherein the biological sample is a blood sample.

3. The method of claim 1, wherein the expression level is measured from an immune cell in the biological sample.

4. The method of claim 3, wherein the immune cell is a CD14 monocyte, CD16 monocyte, a type II conventional dendritic cell (cDC2), a B cell, a plasmablast, a CD4 T-cell with cytotoxic activity (CD4 CTL), a CD8 T-cell, a natural killer (NK) cell, a NK proliferating cell, or a mucosal-associated invariant T (MAIT) cell.

5. The method of claim 4, wherein
(i) the immune cell is a CD14 monocyte and the at least two genes are selected from IFITM1, IFITM3, JUNB, IFI27, LGALS1, MAFB, NFKBIA, and CEBPD;
(ii) the immune cell is a CD16 monocyte and the at least two genes are selected from IFITM2, IFI30, HLA-DPB1, IFI27, HLA-DRB5, CD52, KLF2, and HLA-DQA1; or
(iii) the immune cell is a cDC2 cell and the at least two genes are selected from OAS1, IFITM3, EGR1, HLA-DRB5, EEF1A1, and RPS4Y1.

6. The method of claim 4, wherein:
(i) the immune cell is a CD8 T-cell and the at least two genes are selected from CTSW, GZMH, MT-CO1, GZMB, GNLY, TMSB4X, TRBV4-1, TRGV9, TPT1, TMSB4X, KLF2, and RPS4Y1;
(ii) the immune cell is a CD4 CTL and the at least two genes are selected from TMSB4X, YPEL3, H1FX, KLF2, RPS4Y1, CDKN1A, CTSW, RPS2, ISG20, IFITM1, IFIT3, LY6E, XAF1, IFI6, and ISG15;
(iii) the immune cell is an NK cell and the at least two genes are selected from CCL4, PTGDS, ISG15, GZMB, IFI6, and GNLY; or
(iv) the immune cell is a B cell and the at least two genes are selected from IFIT3, IFITM1, TSC22D3, SESN1, KLF2, HLA-DRB5, MALAT1, H1FX, IGHG1, LA-DQB1, HLA-DRB5, ISG15, IFI6, and TXNDC5.

7. The method of claim 4, wherein is a B intermediate cell, a B memory cell, or a B naïve cell.

8. The method of claim 1, wherein the subject is hospitalized.

9. The method of claim 1, wherein the reference sample is derived from a healthy control subject or a subject having survived critical COVID.

10. The method of claim 1, further comprising administering a treatment based at least in part on the gene expression score.

11. A method for monitoring critical COVID-19 in a subject in need thereof, the method comprising:
obtaining a biological sample from the subject;
measuring a first expression level of at least two genes selected from:
APOBEC3A, AREG, B2M, BST2, CCL4, CD52, CD74, CDKN1A, CEBPB, CEBPD, CFD, CRIP1, CTSW, CYBA, DDIT4, DDIT4, EEF1A1, EEF1B2, EEF1G, EGR1, EIF3L, EMP3, EPSTI1, GNLY, GZMB, GZMH, GZMI, H1FX, H3F3B, HIST1H1E, HLA-A, HLA-C, HLA-DPB1, HLA-DQA1, HLA-DQB1, HLA-DRB5, HSPB1, IER2, IFI27, IFI44L, IFI6, IFIT3, IFITM1, IFITM2, IFITM3, IGHG1, IL7R, IRF1, IRF7, ISG1, ISG15, ISG20, JUN, JUNB, KLF2, KLF6, LGALS1, LGALS9, LY6E, MAFB, MALAT1, MT2A, MT-CO1, MX1, NFKBIA, OAS1, PLAC3, PNRC1, PPP1R15A, PRF1, PTGDS, RHOB, RPS2, RPS4Y1, SAT1, SESN1, TMSB4X, TPT1, TRBV4-1, TRGV9, TSC22D3, TXNDC5, TXNIP, UBE2L6, XAF1, XCL2, YPEL3, or ZFP36;
computing a first gene expression score from the first expression level of the at least two genes; and
subsequently measuring a second expression level of the at least two genes and computing a second gene expression score from the second expression level of the at least two genes;
wherein a change in gene expression score is computed as a difference between the first expression score and the second expression score, and indicates a change in the subject's risk of mortality from critical COVID-19.

12. The method of claim 11, wherein the biological sample is a blood sample.

13. The method of claim 11, wherein the expression level is measured in an immune cell in the biological sample.

14. The method of claim 13, wherein the immune cell is a CD14 monocyte, CD16 monocyte, a type II conventional dendritic cell (cDC2), a B cell, a plasmablast, a CD4 T-cell with cytotoxic activity (CD4 CTL), a CD8 T-cell, a natural killer (NK) cell, a NK proliferating cell, or a mucosal-associated invariant T (MAIT) cell.

15. The method of claim 14, wherein
(i) the immune cell is a CD14 monocyte and the at least two genes are selected from IFITM1, IFITM3, JUNB, IFI27, LGALS1, MAFB, NFKBIA, and CEBPD;
(ii) the immune cell is a CD16 monocyte and the at least two genes are selected from IFITM2, IFI30, HLA-DPB1, IFI27, HLA-DRB5, CD52, KLF2, and HLA-DQA1; or
(iii) the immune cell is a cDC2 cell and the at least two genes are selected from OAS1, IFITM3, EGR1, HLA-DRB5, EEF1A1, and RPS4Y1.

16. The method of claim 4, wherein:
(i) the immune cell is a CD8 T-cell and the at least two genes are selected from CTSW, GZMH, MT-CO1, GZMB, GNLY, TMSB4X, TRBV4-1, TRGV9, TPT1, TMSB4X, KLF2, and RPS4Y1;
(ii) the immune cell is a CD4 CTL and the at least two genes are selected from TMSB4X, YPEL3, H1FX, KLF2, RPS4Y1, CDKN1A, CTSW, RPS2, ISG20, IFITM1, IFIT3, LY6E, XAF1, IFI6, and ISG15;
(iii) the immune cell is an NK cell and the at least two genes are selected from CCL4, PTGDS, ISG15, GZMB, IFI6, and GNLY; or
(iv) the immune cell is a B cell and the at least two genes are selected from IFIT3, IFITM1, TSC22D3, SESN1, KLF2, HLA-DRB5, MALAT1, H1FX, IGHG1, HLA-DQB1, HLA-DRB5, ISG15, IFI6, and TXNDC5.

17. The method of claim 14, wherein the B cell is a B intermediate cell, a B memory cell, or a B naïve cell.

18. The method of claim 11, wherein the subject is hospitalized.

19. The method of claim 11, further comprising administering a treatment to the subject based at least in part on the change in the subject's risk of mortality from critical COVID-19.

20. The method of claim 11, further comprising administering a treatment to the subject based at least in part on the change in gene expression score.
